(19) United States
(12) Patent Application Publication
(10) Pub. No.: US 2011/0053947 A1
(43) Pub. Date: Mar. 3, 2011

(54) Arylchalcogenoarylalkyl-substituted imidazolidine-2,4-diones, process for preparation thereof, medicaments comprising these compounds and use thereof

(75) Inventors: Gerhard JAEHNE, Frankfurt (DE); Siegfried STENGELIN, Eppstein-Bremthal (DE); Matthias GOSSEL, Hofheim (DE); Thomas KLABUNDE, Frankfurt (DE); Irvin WINKLER, Liederbach (DE)

(73) Assignee: SANOFI-AVENTIS, Paris (FR)

(21) Appl. No.: 12/852,216

(22) Filed: Aug. 6, 2010

Related U.S. Application Data
(63) Continuation of application No. PCT/EP2009/000592, filed on Jan. 30, 2009.

(30) Foreign Application Priority Data
Feb. 7, 2008 (EP) 08290130.7

(51) Int. Cl. A61K 31/4985; C07D 241/04; C07D 233/40; C07D 401/04; A61K 31/497; A61K 31/4166; A61K 31/4439; A61P 25/18; A61P 25/28

(52) U.S. Cl. 514/250; 544/383; 548/320.5; 548/317.1; 546/274.1; 514/254.05; 514/391; 514/341

(57) ABSTRACT

The invention relates to compounds of formula (I) wherein the groups R and R′, A, D, E, G, L, p and R1 to R10 have the stated meanings and to their physiologically compatible salts. Said compounds are suitable, for example, as anti-obesity drugs.

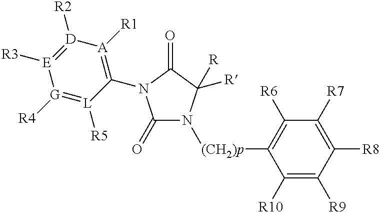

The invention relates to imidazolidine-2,4-diones which are substituted by an aralkyl radical and to the physiologically compatible salts thereof.

Structurally similar imidazoline-2,4-diones have already been described (U.S. Pat. No. 5,411,981). Structurally similar nitrogen-containing heterocyclic derivatives have also been described in US 2007/0010529; these compounds are suitable for the treatment of inflammatory disorders.

It was an object of the invention to provide compounds which display a therapeutically utilizable action. In particular, it was an object of the invention to find novel compounds which are suitable for the treatment of metabolic syndrome, of type II diabetes and of obesity.

The invention therefore relates to compounds of the formula I

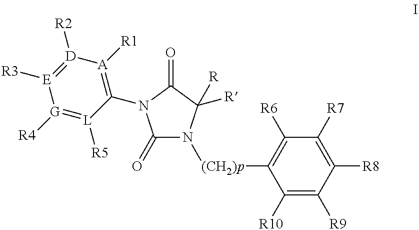

in which
R, R′ are each independently H, (CH₂)ₙ-aryl, (C₁-C₆)-alkyl, where (C₁-C₆)-alkyl may be substituted by halogen, O—R14, S(O)ₘ—R12 or NR13R15;
or R and R′ together form a ring having from three to eight carbon atoms, where one carbon atom may be replaced by O, S(O)ₘ, NR13 or NR15;
m is 0, 1, 2;
n is 0, 1, 2, 3, 4;
p is 1, 2, 3, 4, 5;
q is 1, 2, 3, 4;
r is 2, 3, 4, 5, 6;
v is 0, 1, 2, 3, 4;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, N₃, NC, NO₂, CF₃, (C₁-C₈)-alkyl, (C₃-C₈)-cycloalkyl, (CH₂)_{q}-[(C₃-C₈)-cycloalkyl], (CH₂)ₙ-[(C₃-C₈)-cycloalkenyl], (CH₂)ₙ-[(C₇-C₁₂)-bicycloalkyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O—R11, NR13R15, NH—CN, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl],
   CO—O[(C₃-C₈)-cycloalkyl], CO—O—(CH₂)ₙ-aryl, CO—O—(CH₂)ₙ-heteroaryl, CO—NH₂, CO—NH—CN, CO—NH—[(C₁-C₈)-alkyl], CO—N[(C₁-C₈)-alkyl]₂, CO—NH—[(C₃-C₈)-cycloalkyl], CO—N[(C₃-C₈)-cycloalkyl]₂, C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₈)-alkyl, CO—(C₃-C₈)-cycloalkyl, CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O—R12, CH₂—O—(CH₂)ₙ—CO—O[(C₁-C₈)-alkyl], CH₂—O—(CH₂)ₙ—CO—NH₂, CH₂—O—(CH₂)_{q}—COOH, where the alkyl, cycloalkyl, cycloalkenyl, bicycloalkyl and tricycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, OCF₃, OH, O—(CH₂)ₙ-aryl, (CH₂)ₙ-aryl, S(O)ₘ—(C₁-C₆)-alkyl, SO₂—NH₂, SH, NR12R13, NH—CO—[(C₁-C₆)-alkyl], NH—CO—(CH₂)ₙ-aryl, (CH₂)ₙ—COOH, (CH₂)ₙ—CONH₂, (CH₂)ₙ—CO—O(C₁-C₆)-alkyl, (CH₂)ₙ—CO—(C₁-C₆)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
   R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ᵣ—OH, O—R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C1-C8)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂,

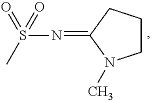

SO₂—NH—CO—R12, SO₂—NHR12, SO₂—NH—(CH₂), —OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)—NH₂, (CH₂)ₙ—C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃,
(C₁-C₈)-alkyl, (C₂-C₁₀)-alkenyl, (C₂-C₁₀)-alkynyl, (C₃-C₈)-cycloalkyl, aryl, heteroaryl,
(CH₂)ₙ—CO—[O—(C₁-C₈)-alkyl], (CH₂)ₙ—CO—[O—(C₃-C₈)-Cycloalkyl], (CH₂)_{b}—CO—[(C₁-C₈)-alkyl], (CH₂)ₙ—CO—[(C₃-C₈)-cycloalkyl],
(CH₂)ₙ—CO—[O—(CH₂)ᵥ-aryl],
(CH₂)ₙ—CO—NH₂, (CH₂)ₙ—COON, (CH₂)ₙ—CO—NH—CN,
(CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂,
(CH₂)ₙ—SO₃H₅ (CH₂)ₙ—SO₂—NH₂,
(CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl],
(C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH,
(CH₂)ₙ—CO—R16,
(CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—CH₂)ₙ—O—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H₅ (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂)ᵣ—OH, O—R13, OCF₃, (CH₂)ₙ—NH₂, (CH₂)ₙ—NH—(C₁-C₈)-alkyl₅ (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂), —SO₂—R16, SO₂—N═CH—N(CH₃)₂,

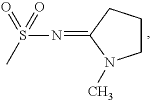

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as T-bicyclic heterocycle, T-aryl or T-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23-C(═NR13)-NR24, NR23-C(═NR22)-NR24, CO—NR23-CR22R23, CO—CR22R23-CO, CR22R23-CO—CR22R24, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CO—NR23-SO₂, SO₂—NR23-CO, CO—CR22R23-SO₂, SO₂—CR22R23-CO, CR23R24-CR23R24-CR23R24, CR23R24-NR23-CR23R24;
R11 is H, (C₁-C₈)-alkyl, (C₂-C₁₀)-alkenyl, (C₂-C₁₀)-alkynyl, (C₃-C₈)-cycloalkyl, (CH₂)_{q}—[(C₃-C₈)-cycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—NH—[(C₃-C₈)-cycloalkyl], (CH₂)ₙ—CO—N[(C₃-C₈)-cycloalkyl]₂, (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21(CONH₂)₂, (CH₂)ₙ—CR21(COOH)₂, (CH₂)ₙ—CR21R22-CO—O[(C₁-C₆)-alkyl], (CH₂)ₙ—CR21R22-CONH₂, (CH₂)ₙ—CR21R22-CO—NH—[(C₁-C₈)-alkyl], (CH₂)ₙ—CR21R22-CO—N[C₁-C₈)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R12 is H, (C₁-C₈)-alkyl, (C₃-C₈)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl,
   where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ—[(C₁-C₆)-alkyl], SO₂—NH₂, COOH, CONH₂, CO—[O(C₁-C₆)-alkyl], CO—(C₁-C₆)-alkyl and where the alkyl radicals may be substituted by fluorine atoms;
R14 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], (CH₂)ₙ—CO—[O—(CH₂)ₙ-aryl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—COOH, where the alkyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R15 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], CO—[(C₁-C₈)-alkyl], CO—[(C₃-C₈)-cycloalkyl], CO-aryl, CO-heteroaryl, (CH₂)ₙ—CO—NH₂, (CH₂)_{q}—COOH, (CH₂)ₙ—SO₂—NH₂, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R16 is aziridin-1-yl, azetidin-1-yl, 3-hydroxyazetidin-1-yl, piperidin-1-yl, pyrrolidin-1-yl, 3-pyrrolidinol-1-yl, morpholin-N-yl, piperazin-1-yl, 4-[(C₁-C₆)-alkyl]piperazin-1-yl, thiomorpholin-4-yl, thiomorpholine-1,1-dioxid-4-yl, NH—(CH₂), —OH, NH—CH(CH₂OH)₂, NH—C(CH₂OH)₃, N[(C₁-C₆)-alkyl-OH]₂, N[(C₁-C₆)-alkyl][(C₁-C₆)-alkyl-OH], D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-CO—O(C₁-C₆)-alkyl, N[(C₁-C₆)-alkyl][(C₁-C₈)-alkyl]-COOH, N[(C₁-C₆)-alkyl][(C₁-C₈)-alkyl]-CONH₂, NH—[C(H)(aryl)]-CO—O(C₁-C₆)-alkyl, NH—[C(H)(aryl)]-COOH, NH—[C(H)(aryl)]-CONH₂, N[(C₁-C₆)-alkyl][C(H)(aryl)]—CO—O(C₁-C₆)-alkyl, N[(C₁-C₆)-alkyl][C(H)(aryl)]-COOH, N[(C₁-C₆)-alkyl][C(H)(aryl)]-CONH₂, NH—[C(H)(heteroaryl)]—CO—O(C₁-C₆)-alkyl, NH—[C(H)(heteroaryl)]-COOH, NH—[C(H)(heteroaryl)]-CONH₂, N[(C₁-C₆)-alkyl][C(H)(heteroaryl)]—CO—O(C₁-C₆)-alkyl, N[(C₁-C₆)-alkyl][C(H)(heteroaryl)]-COOH, N[(C₁-C₆)-alkyl][C(H)(heteroaryl)]-CONH₂, N[(C₁-C₆)-alkyl][(C₃-C₈)-cycloalkyl]-CO—O(C₁-C₆)-alkyl, N[(C₁-C₆)-alkyl][(C₃-C₈)-cycloalkyl]-COOH, N[(C₁-C₆)-alkyl][(C₃-C₈)-cycloalkyl]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(C₁-C₈)-alkyl-OH, NH—[(C₁-C₆)-alkyl]-SO₂—(C₁-C₆)-alkyl, NH—[(C₁-C₆)-alkyl]-SO₃H, NH—[(C₁-C₆)-alkyl]-SO₂—NH₂, N[(C₁-C₆)-alkyl]{[(C₁-C₆)-alkyl]-SO₃H},
   where the alcohol (OH) functions may be replaced by F and where the aryl or heteroaryl radical may be replaced by halogen, CN, (C₁-C₆)-alkyl, O—(C₁-C₆)-alkyl, OH, SO₂—NH₂, COOH, CONH₂, CO—O(C₁-C₆)-alkyl, CO—(C₁-C₆)-alkyl;
R18 is (CH₂)ₙ—CR25R26-(O—O(C₁-C₆)-alkyl, (CH₂)ₙ—CR25R26-CO—NH₂, (CH₂)ₙ—CR25R26-COOH;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
R23, R24 are each independently H, (C₁-C₆)-alkyl, (C₃-C₈)-cycloalkyl, [(C₁-C₆)-alkyl]-[(C₃-C₈)-cycloalkyl], aryl, [(C₁-C₆)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₆)-alkyl radical;
R25, R26 are each independently H, F, (C₁-C₆)-alkyl, aryl, [(C₁-C₆)-alkyl]-aryl, where the aryl may be substituted by halogen, CN, OH, O—(C₁-C₆)-alkyl, or the R25 and R26 radicals, together with the carbon atom bonded to them, form a three- to seven-membered carbocycle in which one carbon atom may be replaced by O, S(O)ₘ, NH, N[(C₁-C₆)-alkyl] or CO;
   excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
   and physiologically compatible salts thereof.

Preference is given to compounds of the formula I in which one or more radicals are each defined as follows:
R, R′ are each independently H, (CH₂)ₙ-aryl, (C₁-C₆)-alkyl, where (C₁-C₆)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2, 3;
p is 1, 2, 3, 4;
q is 1, 2, 3;
r is 2, 3, 4, 5;
v is 0, 1, 2, 3;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, (CH₂)_{q}-[(C₃-C₈)-cycloalkyl], (CH₂)ₙ—[(C₇-C₁₂)-bicycloalkyl], (CH₂)ₙ—[(C₇-C₁₂)-tricycloalkyl], adamantan-1-yl, adamantan-2-yl, (CH₂)ₙ-aryl, (CH₂)ₙ-heteroaryl, OCF₃, O—R11, NR13R15, NH—CN, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₈)-cycloalkyl], CO—O—(CH₂)ₙ-aryl, CO—O—(CH₂)ₙ-heteroaryl, CO—NH₂, CO—NH—CN, CO—NH—[(C₁-C₈)-alkyl], CO—N[(C₁-C₈)-alkyl]₂, CO—NH—[(C₃-C₈)-cycloalkyl], C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₈)-alkyl, CO—(C₃-C₈)-cycloalkyl, CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O—R12, CH₂—O—(CH₂)_{q}—COOH, where the alkyl, cycloalkyl, cycloalkenyl, bicycloalkyl and tricycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, OCF₃, OH, O—(CH₂)ₙ-aryl, (CH₂)ₙ-aryl, S(O)ₘ—(C₁-C₆)-alkyl, SO₂—NH₂, SH, NR12R13, NH—CO—[(C₁-C₆)-alkyl], NH—CO—(CH₂)ₙ-aryl, (CH₂)ₙ—COOH, (CH₂)ₙ—CONH₂, (CH₂)ₙ—CO—O(C₁-C₆)-alkyl, (CH₂)ₙ—CO—(C₁-C₆)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
   R11, F, Cl, Br, I, CN, N₃, NC, NO₂, CF₃, (CH₂)ₙ—O—R11, (CH₂)ₙ—O—(CH₂), —OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂), —OH, O—R13, OCF₃, (CH₂)ₙ—O—(CH₂), —NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂), —NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—NH—[(C₁-C₈)-alkyl], (CH₂)ₙ, —NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂,

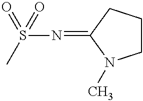

SO₂—NH—CO—R12, SO₂—NHR12, SO₂—NH—(CH₂), —OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)NH₂, (CH₂)ₙ—C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂), —OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂), —OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NFI[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣOH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂), —SO₂—R16, SO₂—N═CH—N(CH₃)₂,

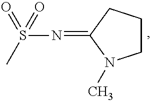

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)—OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₈)-alkyl, SO₂—NH₂, COOH, CONH₂, CO—[O(C₁-C₆)-alkyl], and where the alkyl radicals may be substituted by fluorine atoms;
   where at least one of the R6, R7, R8, R9 and R10 radicals is always defined as T-bicyclic heterocycle, T-aryl or T-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23-C(═NR13)-NR24, NR23-C(═NR22)—NR24, CO—NR23-CR22R23, CO—CR22R23-CO, CR22R23-CO—CR22R24, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CO—NR23-SO₂, SO₂—NR23-CO, CO—CR22R23-SO₂, SO₂—CR22R23-CO, CR23R24-CR23R24-CR23R24, CR23R24-NR23-CR23R24;
R11 is H, (C₁-C₈)-alkyl, (C₂-C₁₀)-alkenyl, (C₂-C₁₀)-alkynyl, (C₃-C₈)-cycloalkyl, (CH₂)_{q}-[(C₃-C₈)-cycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}CO—NH₂, (CH₂)_{q}—COOH, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21(CONH₂)₂, (CH₂)ₙ—CR21(COOH)₂, (CH₂)ₙ—CR21R22-CO—O—[(C₁-C₆)-alkyl], (CH₂)ₙ—CR21R22-CONH₂, (CH₂)ₙ—CR21R22-CO—NH—[(C₁-C₈)-alkyl], (CH₂)ₙ—CR21R22-CO—N[(C₁-C₈)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl, and where the alkyl radicals may be substituted by fluorine atoms;
R12 is H, (C₁-C₈)-alkyl, (C₃-C₆)-cycloalkyl, (CH₂)ₙ—[(C₇-C₁₀)-tricycloalkyl], (CH₂)ₙ-aryl, (CH₂)ₙ-heteroaryl, where the alkyl, cycloalkyl, bicycloalkyl or tricycloalkyl radicals may be substituted by fluorine atoms, and where the aryl or heteroaryl radical may be substituted by halogen, CN, (C₁-C₃)-alkyl, O—(C₁-C₃)-alkyl, SO₂—NH₂, COOH, CONH₂, CO—O(C₁-C₃)-alkyl, CO—(C₁-C₃)-alkyl and where the alkyl radicals may be substituted by fluorine atoms;
R13 is H, SO₂—[(C₁-C₆)-alkyl], SO₂—[(C₃-C₆)-cycloalkyl], SO₂—(CH₂)ₙ-aryl, SO₂—(CH₂)ₙ-heteroaryl,
   where the alkyl and cycloalkyl radicals may be substituted by fluorine atoms and where the aryl or heteroaryl radical may be substituted by halogen, CN, (C₁-C₃)-alkyl, (C₃-C₆)-cycloalkyl, O—[(C₁-C₃)-alkyl], S(O)ₘ—[(C₁-C₃)-alkyl], SO₂—NH₂, COOH, CONH₂, CO—[O(C₁-C₃)-alkyl], CO—(C₁-C₃)-alkyl, and where the alkyl radicals may be substituted by fluorine atoms;
R15 is (C₁-C₈)-alkyl,
   where the alkyl radical may be substituted by fluorine atoms;
R16 is aziridin-1-yl, azetidin-1-yl, 3-hydroxy-azetidin-1-yl, piperidin-1-yl, pyrrolidin-1-yl, 3-pyrrolidinol-1-yl, morpholin-N-yl, piperazin-1-yl, 4-[(C₁-C₆)-alkyl]piperazin-1-yl, thiomorpholin-4-yl, thiomorpholine-1,1-dioxid-4-yl, NH₄—CH₂)ᵣ—OH, NH—CH(CH₂OH)₂, NH—C(CH₂OH)₃, N[(C₁-C₆)-alkyl-OH]₂, D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-COOH, NH—[C(H)(aryl)]-CO—O(C₁-C₆)-alkyl, NH-[C(H)(aryl)]-COOH, NH—[C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(C₁-C₆)-alkyl-OH, NH—[(C₁-C₆)-alkyl]-SO₂—(C₁-C₆)-alkyl, NH—[(C₁-C₆)-alkyl]-SO₃H, NH—[(C₁-C₆)-alkyl]-SO₂—NH₂,
   where the alcohol (OH) functions may be replaced by F and where the aryl or heteroaryl radical may be replaced by halogen, CN, (C₁-C₆)-alkyl, O—(C₁-C₆)-alkyl, OH, SO₂—NH₂, COOH, CONH₂, CO—O(C₁-C₆)-alkyl, CO—(C₁-C₆)-alkyl;
R18 is (CH₂)ₙ—CR25R26-(O—O(C₁-C₄)-alkyl, (CH₂)ₙ—CR25R26-CO—NH₂, (CH₂)ₙ—CR25R26-COOH;
R20 is H, (C₁-C₃)-alkyl, (C₃-C₆)-cycloalkyl, aryl, [(C₁-C₆)-alkyl]-aryl;
R21 is H, F, CF₃, (C₁-C₃)-alkyl, (C₃-C₆)-cycloalkyl, OH, O—(C₁-C₃)-alkyl, O—(C₃-C₆)-cycloalkyl, O—(CH₂)ₙ-aryl, O—(CO)—(C₁-C₃)-alkyl, O—(CO)—(C₃-C₆)-cycloalkyl, O—(CO)—O—(C₁-C₃)-alkyl, O—(CO)—O—(C₃-C₆)-cycloalkyl, NH₂, NH—[(C₁-C₃)-alkyl]-aryl, NH—(C₁-C₃)-alkyl, NH—(CO)—(C₁-C₃)-alkyl;
R22 is H, CF₃, (C₁-C₃)-alkyl, aryl, [(C₁-C₆)-alkyl]-aryl;
R23, R24 are each independently H, (C₁-C₆)-alkyl, (C₃-C₈)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
   excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
   and the physiologically compatible salts thereof.

Particular preference is given to compounds of the formula I in which one or more radicals are each defined as follows:
R, R′ are each independently H, (CH₂)ₙ-aryl, (C₁-C₆)-alkyl where (C₁-C₆)-alkyl or the aryl radical may be substituted by halogen;
or R and R′ together form a ring having three to eight carbon atoms where one carbon atom may be replaced by O, S(O)ₘ, NR13 or NR15;
m is 0, 1, 2;
n is 0, 1, 2;
P is 1, 2, 3;
q is 1, 2;
r is 2, 3, 4;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, adamantan-1-yl, adamantan-2-yl, (CH₂)ₙ-aryl, (CH₂)ₙ-heteroaryl, OCF₃, O—R11, NR13R15, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₆)-cycloalkyl], CO—NH₂, CO—NH—[(C₁-C₄)-alkyl], CO—N[(C₁-C₄)-alkyl]₂, CO—NH—[(C₃-C₆)-cycloalkyl], C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH,
   where the alkyl, cycloalkyl radicals may be substituted by fluorine atoms, and where the aryl or heteroaryl radicals may be substituted by halogen, CN, (C₁-C₄)-alkyl, (C₃-C₆)-cycloalkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
   R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ₙ—OH, O—R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣOH, (CH₂)ₙ—NH—(CH₂)ₙ—CO—N [(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂ (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂,

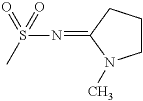

SO₂—NH—CO—R12, SO₂—NHR12, SO₂—NH—(CH₂)ᵣ—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)NH₂, (CH₂)ₙ—C(═NH)—NHOH, (CH₂)—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂; (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl, and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂), —OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl₅ (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂,

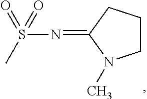

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)—OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl],
where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)alkyl and where the alkyl radicals may be substituted by fluorine atoms;
   where at least one of the R6, R7, R8, R9 and R10 radicals is always defined as T-bicyclic heterocycle, T-aryl or T-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23—C(═NR13)-NR24, NR23-C(═NR22)—NR24, CO—NR23-CR22R23, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CR23R24-CR23R24-CR23R24, CR23R24-NR23-CR23R24;
R11H, (C₁-C₈)-alkyl, (C₂-C₁₀)-alkynyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₆)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₄)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22-CO—O[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms;
R12 is H, (C₁-C₈)-alkyl, (C₃-C₈)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl
   where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl] and where the alkyl radicals may be substituted by fluorine atoms;
R15 is (C₁-C₆)-alkyl,
   where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidinol-1-yl, morpholin-N-yl, piperazin-1-yl, 4-[(C₁-C₆)-alkyl]piperazin-1-yl, thiomorpholine-1,1-dioxid-4-yl, NH—(CH₂), —OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-alkyl][C₁-C₄)-alkyl]-COOH, NH—[C(H)(aryl)]-CO—O(C₁-C₄)-alkyl, NH—[C(H)(aryl)]-COOH, NH—[C(H)(aryl)]-CONH₂, NH—[C(H)(heteroaryl)]-CO—O(C₁-C₄)-alkyl, NH—[C(H)(heteroaryl)]-COOH, NH—[C(H)(heteroaryl)]-CONH₂, NH—[(C₃-C₆)-cycloalkyl]-CO—O(C₁-C₄)-alkyl, NH—[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
   where the alcohol (OH) functions may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 is (CH₂)ₙ—CR25R26-CO—O(C₁-C₄)-alkyl, (CH₂)ₙ—CR25R26-CO—NH₂, (CH₂)ₙ—CR25R26-COOH;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R23, R24 are each independently H, (C₁-C₄)-alkyl, (C₃-C₆)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
   excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
   and the physiologically compatible salts thereof.

Very particular preference is given to compounds of the formula I in which one or more radicals are each defined as follows:
R, R′ are each independently H, aryl, (C₁-C₄)-alkyl, where (C₁-C₄)-alkyl or the aryl radical may be substituted by halogen; or R and R′ together form a ring having from three to eight carbon atoms, where one carbon atom may be replaced by O, S(O)ₘ, NR13 or NR15;
m is 0, 1, 2;
n is 0, 1, 2;
P is 1, 2, 3;
q is 1, 2;
r is 2, 3;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, (CH₂)ₙ-aryl, (CH₂)ₙ-heteroaryl, OCF₃, O—R11, NR13R15, S(O)ₘ—R12, SO₂—NH₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₈)-cycloalkyl], CO—NH₂, CO—NH—[(C₁-C₄)-alkyl], CO—N[(C₁-C₄)-alkyl]₂, C(═NH)—NH₂, C(═NH)—R16, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH, where the alkyl, cycloalkyl radicals may be substituted by fluorine atoms, and where the aryl or heteroaryl radicals may be substituted by halogen, CN, (C₁-C₄)-alkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
   R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O—R13, OCF₃, (CH₂)ₙ—O—(CH₂)ᵣ—NH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂), —NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—N [(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂,

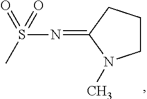

SO₂—NH—CO—R12, SO₂—NHR12, SO₂—NH—(CH₂)—OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)—NH₂, (CHA-C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂), —OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH— (CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂), —NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂), —NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)_{I}—S(O)ₘ—(C₁-C₈)-alkyl, (CH₂)_{I}—S(O)ₘ—(C₃-C₈)-CyCiOalkyl, (CH₂)ₙ—SO₂—R16, SO₂—N═CH—N(CH₃)₂,

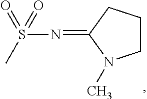

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂)ᵣ—OH, SO₂—NH—(CH₂)ᵣ—NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
   where at least one of the R6, R7, R8, R9 and R10 radicals is always defined as T-bicyclic heterocycle, T-aryl or T-heteroaryl and
   where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T NR23-CO—NR24, NR23-SO₂—NR24, SO₂—NR23-SO₂, CO—NR23-CO, NR23-C(═NR13)-NR24, NR23-C(═NR22)-NR24, CO—NR23-CR22R23, NR23-SO₂—CR22R24, CR22R24-SO₂—NR23, CR22R23-NR23-SO₂, SO₂—CR22R23-NR23, SO₂—NR23-CR22R23-, NR23-CR22R23-SO₂, CR23R24-CR23R24-CR23R24;
R11 H, (C₁-C₈)-alkyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₄)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22-CO—O—[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH,
   where the alkyl, alkeny, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl,
   where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl] and where the alkyl radicals may be substituted by fluorine atoms;
R15 (C₁-C₆)-Alkyl,
   where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholine-1,1-dioxid-4-yl, NH—(CH₂)ᵣ—OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-Alkyl][(C₁-C₄)-Alkyl]-COOH, NH—[C(H)(aryl)]-CO—O(C₁-C₄)-alkyl, NH—[C(H)(aryl)]-COOH, NH—[C(H)(aryl)]-CONH₂, NH—[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH—[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
   where the alcohol (OH) functionalities may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 (CH₂)ₙ—CR25R26-CO—O(C₁-C₄)-alkyl, (CH₂)ₙ—CR25R26-CO—NH₂, (CH₂)ₙ—CR25R26-COOH;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R23, R24 are each independently H, (C₁-C₄)-alkyl, (C₃-C₆)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
   excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
   and the physiologically compatible salts thereof.

Preference is further given to compounds of the formula Ia

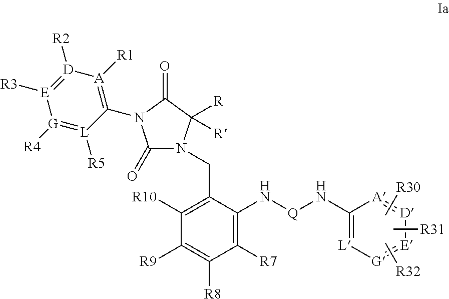

in which
R, R′ are each independently H, aryl, (C₁-C₄)-alkyl, where (C₁-C₄)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2;
q is 1, 2;
r is 2, 3;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 is defined as S or O and where the five- or six-membered ring may be fused to —(CH₂)₃— or —(CH₂)₄— or —CH═CH—CH═CH— to form a bicyclic system;
Q is C═O, SO₂;
R1, R2, R3, R4, R5 are each independently H, F, Cl, Br, I, CN, CF₃, (C₁-C₈)-alkyl, (C₃-C₈)-cycloalkyl, (CH₂)ₙ-aryl, (CH₂)ₙ-heteroaryl, OCF₃, O—R11, NR13R15, S(O)ₘ—R12, SO₂—NH₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₆)-cycloalkyl], CO—NH₂, CO—NH—[(C₁-C₄)-alkyl], CO—N[(C₁-C₄)-alkyl]₂, C(═NH)—NH₂, C(═NH)—R16, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH, where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R7, R8, R9, R10 are each independently H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, Aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-Cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂), —OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂), —OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂₅(CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂₅(CH₂)ₙ—NH—C(═NH)—R16, (CH₂)ₙ—NH—C(═NH)—NH[(C₁-C₈)-Alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂), —OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₙᵣ(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂,

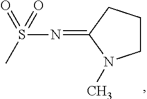

(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl, and where the alkyl radicals may be substituted by fluorine atoms;
   where one of the three radical pairs of R7 and R8, or R8 and R9, or R9 and R10 may in each case together form the —CH₂—CH₂—CH₂— or —CH₂—CH₂—CH₂—CH₂— groups in which up to two —CH₂ groups may be replaced by —O— and where the —CH₂—CH₂—CH₂— or —CH₂—CH₂—CH₂—CH₂-groups may be substituted by F, (C₁-C₈)-alkyl or ═O;
R11 is H, (C₁-C₈)-alkyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₄)-alkyl], (C₂-C₆)-alkynyl-CONH₂, (C₂-C₆)-alkynyl-COOH, (CH₂)ₙ—CR21[(C₁-C₄)-alkyl)]₂, (CH₂)ₙ—CR21(CONH₂)₂, (CH₂)ₙ—CR21(COOH)₂, (CH₂)ₙ—CR21R22-CO—O—[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alk_{yl]}₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms, and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl,
   where the alkyl and cycloalkyl radicals may be substituted by fluorine atoms, and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl], and where the alkyl radicals may be substituted by fluorine atoms;
R15 is (C₁-C₆)-alkyl,
   where the alkyl radical may be substituted by fluorine atoms;
R16 is aziridin-1-yl, az etidin-1-yl, 3-hydroxyazetidin-1-yl, piperidin-1-yl, pyrrolidin-1-yl, 3-pyrro lidino1-1-yl, morpholin-N-yl, piperazin-1-yl, 4-[(C₁-C₆)-alkyl]piperazin-1-yl, thiomorpholin-1,1-dioxid-4-yl, NH—(CH₂)ᵣ—OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-alkyl][(C₁-C₄)-alkyl]-COOH, NH—[C(H)(aryl)]-CO—O(C₁-C₄)-alkyl, NH—[C(H)(aryl)]-COOH, NH—[C(H)(aryl)]-CONH₂, NH—[C(H)(heteroaryl)]-CO—O(C₁-C₄)-alkyl, NH—[C(H)(heteroaryl)]-COOH, NH—[C(H)(heteroaryl)]-CONH₂, NH—[(C₃-C₆)-cycloalkyl]-CO—O(C₁-C₄)-alkyl, NH—[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
   where the alcohol (OH) functions may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 is (CH₂)ₙ—CR25R26-CO—O(C₁-C₄)-alkyl, (CH₂)ₙ—CR25R26-CO—NH₂, (CH₂)ₙ—CR25R26-COOH;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
A′, D′, E′, G′, L′ are each independently CH or N, where, when they are defined as N, the corresponding substituent R30, R31 or R32 is absent if it would be bonded to the nitrogen atom;
R30, R31, R32 are each independently R11, F, Cl, Br, I, CN, CF₃, (CH₂)ₙ—O—R11, O—R13, OCF₃, (CH₂)ₙ—NH—R11, (CH₂)ₙ—NRCH₂)_{q}—CO—O(C₁-C₄)-alkylh, (CH₂)ₙ—NRCH₂)_{q}—COOK, (CH₂)ₙ—N[(CH₂)_{q}—CONH₂]₂, (CH₂)ₙ—NH—R13, (CH₂)ₙ—N(R13)₂, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NH—(CH₂)ₙ—CO—NH—[(C₁-C₄)-alkyl], (CH₂)ₙ—NH—(CH₂)ₙ—CO—N[(C₁-C₄)-alkyl]₂, (CH₂)ₙ—NH—C(CH₃)₂—CO—O(C₁-C₄)-alkyl, (CH₂)ₙ—NH—C(CH₃)₂—CO—O(C₃-C₆)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₄)-alkyl], (CH₂)ₙ—NH—C(CH₃)₂—CO—N[(C₁-C₄)-alkyl]₂, (CH₂)ₙ—NH—C(CH₃)₂—COOH, S(O)ₘ—R12, SO₂—R16, SO₂—N═CH—N(CH₃)₂, SO₂—NH—CO—R12, SO₂—NHR12, SO₂—N[(C₁-C₄)-alkyl]₂, SF₅, COOH, CO—NH₂, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—C(═NH)—NHOH, (CH₂)ₙ—C(═NR13)NHR12, (CH₂)ₙ—C(═NR12)NR12R13, (CH₂)ₙ—C(═NSO₂—R12)NH₂, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms;
   excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
   and the physiologically compatible salts thereof.

Preference is further given to compounds of the formula Ia in which
R, R′ are each (C₁-C₄)-alkyl;
or R and R′ together form a ring having from three to eight carbon atoms;
m is 0, 1, 2;
n is 0, 1, 2;
A, D, E, G, L are each C;
Q is C═O, SO₂;
R1, R2, R3, R4, R5 are each independently H, F, Cl, Br, CN, CF₃, (C₁-C₈)-cycloalkyl, O-phenyl;
R7, R8, R9, R10 are each independently H, F, Cl, Br, CF₃, —OCH₃;
A′, E′ are each independently CH or N, where, when they are defined as N, the corresponding substituent R30, R31 or R32 is absence if it would be bonded to the nitrogen atom,
R30, R31, R32 are each independently H, F, Cl, Br, CF₃, OH, NO₂, NH₂, CONH₂, COOH, —COO—(C₁-C₈)-Alkyl, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, S(O)ₘ—(C₁-C₄)-alkyl, S(O)ₘ—(CH₂)—COOH, S(O)ₘ—(CH₂)—COO—(C₁-C₄)-alkyl, SO₂—Cl, SO₂—NH₂, SO₃H;
   and the physiologically compatible salts thereof.

Preference is further given to compounds of the formula Ia, in which
R, R′ are each (C₁-C₄)-alkyl;
or R and R′ together form a ring having from three to eight carbon atoms;
m is 0, 1, 2;
n is 0, 1, 2;
A, D, E, G, L are each C;
Q is C═O, SO₂;
R1, R2, R3, R4, R5 are each independently H, F, Cl, Br, CN, CF₃, (C₁-C₈)-cycloalkyl, O-phenyl;
R7, R8, R9, R10 are each independently H, F, Cl, Br, CF₃, —OCH₃;
A′, E′ are each independently CH or N, where, when they are defined as N, the corresponding substituent R30, R31 or R32 is absent if it would be bonded to the nitrogen atom,
R30, R31, R32 are each independently H, F, Cl, Br, CF₃, OH, NO₂, NH₂, CONH₂, COOH, —COO—(C₁-C₈)-Alkyl, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, S(O)ₘ—(C₁-C₄)-alkyl, S(O)ₘ—(CH₂)—COOH, S(O)ₘ—(CH₂)—COO—(C₁-C₄)-alkyl, SO₂—Cl, SO₂—NH₂, SO₃H;
   and the physiologically compatible salts thereof.

In one embodiment, preference is given to compounds of the formula I in which p is 1.

In one embodiment, preference is given to compounds of the formula I in which T is —NH—CO—NH—.

In one embodiment, preference is given to compounds of the formula I in which T is —NH—SO₂—NH—.

In one embodiment, preference is given to compounds of the formula I in which R and R′ are each methyl.

In one embodiment, preference is given to compounds of the formula I in which A, D, E, G and L are each substituted or unsubstituted C (carbon).

In one embodiment, preference is given to compounds of the formula I in which one of the R1, R2, R3, R4 and R5 radicals is not H.

In one embodiment, preference is given to compounds of the formula I in which two of the R1, R2, R3, R4 and R5 radicals are not H.

In one embodiment, preference is given to compounds of the formula I in which two of the R3 radicals are CN or F.

In one embodiment, preference is given to compounds of the formula I in which R4 is CF₃ or cyclopropyl.

In one embodiment, preference is given to compounds of the formula Ia in which one of the R30, R31 and R32 radicals is not H.

When radicals or substituents (for example R12) can occur more than once in the compounds of the formula I, they may all each independently be defined as specified and be the same or different.

The invention further provides both stereoisomer mixtures of the formula I and the pure stereoisomers of the formula I, and also diastereoisomer mixtures of the formula I and the pure diastereoisomers. The mixtures are separated, for example, by a chromatographic route.

The invention relates to compounds of the formula I in the form of their tautomers, racemates, racemic mixtures, stereoisomer mixtures, pure stereoisomers, diastereoisomer mixtures, pure diastereoisomers. The mixtures are separated, for example, by a chromatographic route.

The alkyl radicals in the substituents R1 to R26 and R and R′ may be either straight-chain or branched.

Owing to their high water solubility, pharmaceutically acceptable salts are particularly suitable for medical applications compared to the starting or base compounds. These salts must have a pharmaceutically acceptable anion or cation. Suitable pharmaceutically acceptable acid addition salts of the inventive compounds are salts of inorganic acids, such as hydrochloric acid, hydrobromic acid, phosphoric acid, metaphosphoric acid, nitric acid and sulfuric acid, and also organic acids, for example acetic acid, benzenesulfonic acid, benzoic acid, citric acid, ethanesulfonic acid, fumaric acid, gluconic acid, glycolic acid, isethionic acid, lactic acid, lactobionic acid, maleic acid, malic acid, methanesulfonic acid, succinic acid, p-toluenesulfonic acid and tartaric acid. Suitable pharmaceutically acceptable basic salts are ammonium salts, alkali metal salts (such as sodium and potassium salts), alkaline earth metal salts (such as magnesium and calcium salts), trometamol (2-amino-2-hydroxymethyl-1,3-propanediol), diethanolamine, lysine or ethylenediamine.

Salts with a pharmaceutically unacceptable anion, for example trifluoroacetate, are also included within the scope of the invention as useful intermediates for the preparation or purification of pharmaceutically acceptable salts and/or for use in non-therapeutic applications, for example in vitro applications.

The inventive compounds may also be present in different polymorphic forms, for example as amorphous and crystalline polymorphic forms. All polymorphic forms of the inventive compounds are included within the scope of the invention and are a further aspect of the invention.

Hereinafter, all references to “compound(s) of the formula I” relate to compound(s) of the formula I as described above, and to their salts and solvates as described herein.

An alkyl radical is understood to mean a straight-chain or branched hydrocarbon chain having from one to eight carbons, for example methyl, ethyl, isopropyl, tert-butyl, hexyl, heptyl, octyl. The alkyl radicals may be mono- or polysubstituted as described above.

A cycloalkyl radical is understood to mean a ring system which comprises one or more rings, is present in saturated or partially unsaturated form (with one or two double bonds) and is formed exclusively from carbon atoms, for example cyclopropyl, cyclopentyl, cyclopentenyl, cyclohexyl or adamantyl.

The cycloalkyl radicals may be mono- or polysubstituted by suitable groups as described above.

An aryl radical is understood to mean a phenyl, naphthyl, biphenyl, tetrahydronaphthyl, alpha- or beta-tetralonyl, indanyl or indan-1-onyl radical.

The aryl radicals may be mono- or polysubstituted by suitable groups as described above.

A heteroaryl radical is understood to mean aromatic rings and ring systems which, apart from carbon, also contain heteroatoms, for example nitrogen, oxygen or sulfur. This definition also includes ring systems in which the heteroaryl radical is fused to benzene rings. This likewise includes systems in which one or more CH group(s) has/have been replaced by C═O or C═S, preferably C═O.

Suitable heteroaryl radicals are, for example, furyl, imidazolyl, benzimidazolyl, benzothiazolyl, indolyl, indolinyl, pyrimidinyl, pyridyl, pyrazinyl, pyrrolyl, pyrazolyl, thiazolyl, oxazolyl, isoxazolyl, thienyl, 1,2,3-triazolyl, 1,2,4-triazolyl, tetrazolyl, isoxazolyl, pyridazinyl, 1,3,5-triazinyl, 1,2,4-triazinyl; the 2H-pyridazin-3-one, dihydropyridazine-3,6-dione, imidazolidin-2-one, 1,3-dihydroimidazol-2-one, imidazolidin-2,5-dione, quinoline, isoquinoline, quinoxaline, quinazoline benzo[1,3]dioxole, 2,3-dihydrobenzo[1,4]dioxin, 4H-benzo[1,3]dioxin or 3,4-dihydro-2H-benzo[b][1,4]dioxepine system.

The linkage to the heteroaryl radicals may be at any of the possible atoms; for example, pyridyl may be 2-, 3- or 4-pyridyl; thienyl may be 2- or 3-thienyl; furyl may be 2- or 3-furyl.

Also included are the corresponding N-oxides of these compounds, i.e., for example, 1-oxy-2-, -3- or -4-pyridyl.

The heteroaryl radicals may be mono- or polysubstituted by suitable groups as described above.

The invention also encompasses solvates or hydrates of the compounds of the formula I.

The compounds of the formula I are cannabinoid 1 receptor (CB1R) modulators and are, as such, suitable in humans and in animals for the treatment or for the prevention of diseases which are based on disruption of the endocannabinoid system.

For example, and without restriction, the compounds of the formula I are useful as psychotropic medicaments, especially for the treatment of psychiatric disorders including states of anxiety, depressions, disorders of the mind, insomnia, deliria, obsessive-compulsive neuroses, general psychoses, schizophrenia, attention deficit hyperactivity disorder (ADHD) in hyperkinetic children, and for the treatment of disorders in connection with the use of psychotropic substances, especially in the case of abuse of a substance and/or dependence on such a substance, including alcohol dependence and nicotine dependence, but also dependence on cocaine, methamphetamine and heroin (see, for example, Behavioural Pharmacology 2005, 16:275-296). Reviews of CBR1-mediated means of therapeutic intervention can be found, for example, in Ken Mackie: Annu Rev. Pharmacol. Toxicol. 46, 101-122 (2006), S. C. Black: Curr. Opin. Investig. Drugs 5, 389-394 (2004), V. Di Marzio et al.: Nat. Rev. Drug Discov. 3, 771-784 (2004), B. Le Foll et al.: J. Pharmacol. Exp. Ther. 312, 875-883 (2005) or L. Walter et al.: Br. J. Pharmacol. 141, 775-785 (2004).

The inventive compounds of the formula I may be used as medicaments for the treatment of migraine, stress, disorders of psychosomatic origin, panic attacks, epilepsy, disrupted movement, especially dyskinesias or Parkinson's disease, trembling and dystonia.

The inventive compounds of the formula I can also be used as medicaments for the treatment of disorders of memory, mental defects, especially for the treatment of age-related dementia, of Alzheimer's disease and for the treatment of reduced alertness or wakefulness.

In addition, it is also possible to use the compounds of the formula I as neuroprotectors, for the treatment of ischemia, cranial injuries and the treatment of neurodegenerative disorders, including chorea, Huntington's chorea, Tourette's syndrome.

The inventive compounds of the formula I can also be used as medicaments in the treatment of pain; this includes neuropathic pain, acute peripheral pain, chronic pain of inflammatory origin.

The inventive compounds of the formula I may also serve as medicaments for the treatment of eating disorders (for example binge eating disorders, anorexia and bulimia), for the treatment of addiction to confectionery, carbohydrates, drugs, alcohol or other addictive substances.

The inventive compounds of the formula I are particularly suitable for the treatment of obesity or of bulimia, and for the treatment of type II diabetes and also for the treatment of dyslipidemias and of metabolic syndrome. The inventive compounds of the formula I are therefore useful for the treatment of obesity and of the risks associated with obesity, especially the cardiovascular risks.

Moreover, the inventive compounds may be used as medicaments for the treatment of gastrointestinal disorders, for the treatment of diarrhea, of gastric and intestinal ulcers, of vomiting, of bladder trouble and disorders of urination, of disorders of endocrine origin, of cardiovascular problems, of low blood pressure, of hemmorrhagic shock, of septic shock, chronic liver cirrhosis, liver steatosis, of nonalcoholic steatohepatitis, of asthma, of Raynaud's syndrome, of glaucoma, of fertility problems, termination of pregnancy, early birth, inflammatory symptoms, disorders of the immune system, especially autoimmune and neuroinflammatory disorders, for example rheumatic inflammation of joints, reactive arthritis, of disorders which lead to demyelinization, of multiple sclerosis, of infection disorders and viral disorders, for example encephalitis, ischemic stroke, and as medicaments for chemotherapy of cancer, for the treatment of Guillain-Barré syndrome and for the treatment of osteoporosis.

The inventive compounds of the formula I may also find use as medicaments for the treatment of polycystic ovary syndrome (PCOS).

According to the present invention, the compounds of the formula I are particularly useful for the treatment of psychotic complaints, especially of schizophrenia, reduced alertness and hyperactivity (ADHD) in hyperkinetic children, for the treatment of eating disorders and of obesity, for the treatment of type II diabetes, for the treatment of deficits of memory and cognitive deficits, for the treatment of alcohol addiction, of nicotine addiction, i.e. for alcohol and tobacco withdrawal.

The inventive compounds of the formula I are very particularly useful for the treatment and prevention of eating disorders, appetite disorders, metabolic disorders, gastrointestinal disorders, inflammation symptoms, disorders of the immune system, psychotic disorders, alcohol addiction and nicotine addiction.

According to one of its aspects, the invention relates to the use of a compound of the formula I, the pharmaceutically acceptable salts thereof and the solvates or hydrates thereof for the treatment of the above-specified disorders and diseases.

The compound(s) of the formula I may also be administered in combination with further active ingredients.

The amount of a compound of the formula I which is required in order to achieve the desired biological effect is dependent upon a series of factors, for example the specific compound selected, the intended use, the mode of administration and the clinical condition of the patient. The daily dose is generally in the range from 0.3 mg to 100 mg (typically from 3 mg to 50 mg) per day per kilogram of bodyweight, for example 3-10 mg/kg/day. An intravenous dose may, for example, be in the range from 0.3 mg to 1.0 mg/kg and may suitably be administered as an infusion of from 10 ng to 100 ng per kilogram per minute. Suitable infusion solutions for these purposes may, for example, contain from 0.1 ng to 10 mg, typically from 1 ng to 10 mg, per milliliter. Single doses may contain, for example, from 1 mg to 10 g of the active ingredient. Ampoules for injections may therefore contain, for example, from 1 mg to 100 mg, and single dose formulations which can be administered orally, for example tablets or capsules, may contain, for example, from 1.0 to 1000 mg, typically from 10 to 600 mg. The compounds of the formula I may be used for therapy of the above-mentioned conditions as the compounds themselves, although they are preferably in the form of a pharmaceutical composition with an acceptable carrier. The carrier of course has to be acceptable, in the sense that it is compatible with the other constituents of the composition and is not damaging to the health of the patient. The carrier may be a solid or a liquid or both and is preferably formulated with the compound as a single dose, for example as a tablet, which may contain from 0.05 to 95% by weight of the active ingredient. Further pharmaceutically active substances may likewise be present, including further compounds of the formula I. The inventive pharmaceutical compositions may be produced by one of the known pharmaceutical methods which consist essentially in mixing the constituents with pharmacologically acceptable carriers and/or excipients.

Inventive pharmaceutical compositions are those which are suitable for oral, rectal, topical, peroral (for example sublingual) and parenteral (for example subcutaneous, intramuscular, intradermal or intravenous) administration, although the most suitable mode of administration depends in each individual case on the nature and severity of the condition to be treated and on the type of the compound of the formula I used in each case. Coated formulations and coated slow-release formulations are also encompassed by the scope of the invention. Preference is given to acid- and gastric fluid-resistant formulations. Suitable gastric fluid-resistant coatings include cellulose acetate phthalate, polyvinyl acetate phthalate, hydroxypropylmethylcellulose phthalate and anionic polymers of methacrylic acid and methyl methacrylate.

Suitable pharmaceutical preparations for oral administration may be in the form of separate units, for example capsules, cachets, lozenges or tablets, each of which contains a certain amount of the compound of the formula I; as powder or granules; as solution or suspension in an aqueous or nonaqueous liquid; or as an oil-in-water or water-in-oil emulsion. These compositions may, as already mentioned, be prepared by any suitable pharmaceutical method which includes a step in which the active ingredient and the carrier (which may consist of one or more additional constituents) are brought into contact. In general, the compositions are prepared by uniform and homogeneous mixing of the active ingredient with a liquid carrier and/or finely divided solid carrier, after which the product is shaped if necessary. For example, a tablet can thus be produced by compressing or shaping a powder or granules of the compound, optionally with one or more additional constituents. Compressed tablets can be prepared by tableting the compound in free-flowing form, for example a powder or granules, optionally mixed with a binder, lubricant, inert diluent and/or one (or more) surfactants/dispersants in a suitable machine. Shaped tablets can be prepared by shaping the pulverulent compound moistened with an inert liquid diluent in a suitable machine.

Pharmaceutical compositions which are suitable for peroral (sublingual) administration include lozenges which contain a compound of the formula I with a flavoring, customarily sucrose, and gum arabic or tragacanth, and pastilles which include the compound in an inert base, such as gelatin and glycerol or sucrose and gum arabic.

Suitable pharmaceutical compositions for parenteral administration include preferably sterile aqueous preparations of a compound of the formula I which are preferably isotonic with the blood of the intended recipient. These preparations are preferably administered intravenously, although the administration may also be subcutaneous, intramuscular or intradermal as an injection. These preparations can preferably be produced by mixing the compound with water and making the solution obtained sterile and isotonic with the blood. Injectable compositions according to the invention generally contain from 0.1 to 5% by weight of the active compound.

Suitable pharmaceutical compositions for rectal administration are preferably in the form of single dose suppositories. These can be prepared by mixing a compound of the formula I with one or more conventional solid carriers, for example cocoa butter, and shaping the resulting mixture.

Suitable pharmaceutical compositions for topical application on the skin are preferably in the form of an ointment, cream, lotion, paste, spray, aerosol or oil. Useful carriers include petroleum jelly, lanolin, polyethylene glycols, alcohols and combinations of two or more of these substances. The active ingredient is generally present in a concentration of from 0.1 to 15% by weight of the composition, preferably from 0.5 to 2%.

Transdermal administration is also possible. Suitable pharmaceutical compositions for transdermal applications may be in the form of single plasters which are suitable for long-term close contact with the epidermis of the patient. Such plasters suitably contain the active ingredient in an optionally buffered aqueous solution, dissolved and/or dispersed in an adhesive or dispersed in a polymer. A suitable active ingredient concentration is from approx. 1% to 35%, preferably from approx. 3% to 15%. A particular means of releasing the active ingredient may be by electrotransport or iontophoresis, as described, for example, in Pharmaceutical Research, 2(6): 318 (1986).

Further suitable active ingredients for the combination preparations are:

All antidiabetics which are mentioned in the Rote Liste 2007, chapter 12; all weight-reducing agents/appetite suppressants which are mentioned in the Rote Liste 2007, chapter 1; all diuretics which are mentioned in the Rote Liste 2007, chapter 36; all lipid-lowering agents which are mentioned in the Rote Liste 2007, chapter 58. They can be combined with the compound of the invention of the formula I in particular for a synergistic improvement in action. The active ingredient combination can be administered either by separate administration of the active ingredients to the patient or in the form of combination products in which a plurality of active ingredients are present in one pharmaceutical preparation. If the active ingredients are administered separately, this can be done simultaneously or successively. Most of the active ingredients mentioned hereinafter are disclosed in the USP Dictionary of USAN and International Drug Names, US Pharmacopeia, Rockville 2006.

Antidiabetics include insulin and insulin derivatives, for example Lantus® (see www.lantus.com) or HMR 1964 or Levemir® (insulin detemir), Humalog® (Insulin Lispro), Humulin®, VIAject™, SuliXen® or those as described in WO2005005477 (Novo Nordisk), fast-acting insulins (see U.S. Pat. No. 6,221,633), inhalable insulins, for example Exubera®, Nasulin™, or oral insulins, for example IN-105 (Nobex) or Oral-lyn™ (Generex Biotechnology), or Technosphere® Insulin (MannKind) or Cobalamin™ oral insulin, or insulins as described in WO2007128815, WO2007128817, WO2008034881, WO2008049711, or insulins which can be administered transdermally;

GLP-1 derivatives and GLP-1 agonists, for example exenatide or specific formulations thereof, as described, for example, in WO2008061355, liraglutide, taspoglutide (R-1583), albiglutide, lixisenatide or those which have been disclosed in WO 98/08871, WO2005027978, WO2006037811, WO2006037810 by Novo Nordisk A/S, in WO 01/04156 by Zealand or in WO 00/34331 by Beaufour-Ipsen, pramlintide acetate (Symlin; Amylin Pharmaceuticals), AVE-0010, BIM-51077 (R-1583, ITM-077), PC-DAC:Exendin-4 (an exendin-4 analog which is bonded covalently to recombinant human albumin), CVX-73, CVX-98 and CVx-96 (GLP-1 analog which is bonded covalently to a monoclonal antibody which has specific binding sites for the GLP-1 peptide), CNTO-736 (a GLP-1 analog which is bonded to a domain which includes the Fc portion of an antibody), PGC-GLP-1 (GLP-1 bonded to a nanocarrier), agonists, as described, for example, in D. Chen et al., Proc. Natl. Acad. Sci. USA 104 (2007) 943, those as described in WO2006124529, WO2007124461, WO2008062457, WO2008082274, WO2008101017, WO2008081418, WO2008112939, WO2008112941, WO2008113601, WO2008116294, WO2008116648, WO2008119238, peptides, for example obinepitide (TM-30338), amylin receptor agonists, as described, for example, in WO2007104789, analogs of the human GLP-1, as described in WO2007120899, WO2008022015, WO2008056726, and orally active hypoglycemic ingredients.

Antidiabetics also include agonists of the glucose-dependent insulinotropic polypeptide (GIP) receptor, as described, for example, in WO2006121860.

Antidiabetics also include the glucose-dependent insulinotropic polypeptide (GIP), and also analogous compounds, as described, for example, in WO2008021560.

Antidiabetics also include analogs and derivatives of fibroblast growth factor 21 (FGF-21).

The orally active hypoglycemic ingredients preferably include
sulfonylureas,
biguanidines,
meglitinides,
oxadiazolidinediones,
thiazolidinediones,
PPAR and RXR modulators,
glucosidase inhibitors,
inhibitors of glycogen phosphorylase,
glucagon receptor antagonists,
glucokinase activators,
inhibitors of fructose 1,6-bisphosphatase
modulators of glucose transporter 4 (GLUT4),
inhibitors of glutamine-fructose-6-phosphate amidotransferase (GFAT),
GLP-1 agonists,
potassium channel openers, for example pinacidil, cromakalim, diazoxide, or those as described in R. D. Carr et al., Diabetes 52, 2003, 2513.2518, in J. B. Hansen et al., Current Medicinal Chemistry 11, 2004, 1595-1615, in T. M. Tagmose et al., J. Med. Chem. 47, 2004, 3202-3211 or in M. J. Coghlan et al., J. Med. Chem. 44, 2001, 1627-1653, or those which have been disclosed in WO 97/26265 and WO 99/03861 by Novo Nordisk A/S,
active ingredients which act on the ATP-dependent potassium channel of the beta cells,
inhibitors of dipeptidylpeptidase IV (DPP-IV),
insulin sensitizers,
inhibitors of liver enzymes involved in stimulating gluconeogenesis and/or glycogenolysis,
modulators of glucose uptake, of glucose transport and of glucose reabsorption,
modulators of sodium-dependent glucose transporter 1 or 2 (SGLT1, SGLT2),
inhibitors of 11-beta-hydroxysteroid dehydrogenase-1 (11β-HSD1),
inhibitors of protein tyrosine phosphatase 1B (PTP-1B),
nicotinic acid receptor agonists,
inhibitors of hormone-sensitive or endothelial lipases,
inhibitors of acetyl-CoA carboxylase (ACC1 and/or ACC2) or inhibitors of GSK-3 beta.

Also included are compounds which modify the metabolism, such as active antihyperlipidemic ingredients and active antilipidemic ingredients,
HMGCoA reductase inhibitors,
farnesoid X receptor (FXR) modulators,
fibrates,
cholesterol reabsorption inhibitors,
CETP inhibitors,
bile acid reabsorption inhibitors,
MTP inhibitors,
agonists of estrogen receptor gamma (ERRγ agonists),
sigma-1 receptor antagonists,
antagonists of the somatostatin 5 receptor (SSTS receptor);
compounds which reduce food intake, and
compounds which increase thermogenesis.

In one embodiment of the invention, the compound of the formula I is administered in combination with insulin.

In one embodiment, the compound of the formula I is administered in combination with an active ingredient which acts on the ATP-dependent potassium channel of the beta cells, for example sulfonylureas, for example tolbutamide, glibenclamide, glipizide, gliclazide or glimepiride.

In one embodiment, the compound of the formula I is administered in combination with a tablet which comprises both glimepiride, which is released rapidly, and metformin, which is released over a longer period (as described, for example, in US2007264331, WO2008050987, WO2008062273).

In one embodiment, the compound of the formula I is administered in combination with a biguanide, for example metformin.

In another embodiment, the compound of the formula I is administered in combination with a meglitinide, for example repaglinide, nateglinide or mitiglinide.

In a further embodiment, the compound of the formula I is administered with a combination of mitiglinide with a glitazone, e.g. pioglitazone hydrochloride.

In a further embodiment, the compound of the formula I is administered with a combination of mitiglinide with an alpha-glucosidase inhibitor.

In a further embodiment, the compound of the formula I is administered in combination with antidiabetic compounds, as described in WO2007095462, WO2007101060, WO2007105650.

In a further embodiment, the compound of the formula I is administered in combination with antihypoglycemic compounds, as described in WO2007137008, WO2008020607.

In one embodiment, the compound of the formula I is administered in combination with a thiazolidinedione, for example troglitazone, ciglitazone, pioglitazone, rosiglitazone or the compounds disclosed in WO 97/41097 by Dr. Reddy's Research Foundation, especially 5-[[4-[(3,4-dihydro-3-methyl-4-oxo-2-quinazolinylmethoxy]phenyl]methyl]-2,4-thiazolidinedione.

In one embodiment of the invention, the compound of the formula I is administered in combination with a PPAR gamma agonist, for example rosiglitazone, pioglitazone, JTT-501, G1262570, R-483, CS-011 (rivoglitazone), DRL-17564, DRF-2593 (balaglitazone), INT-131, T-2384, or those as described in WO2005086904, WO2007060992, WO2007100027, WO2007103252, WO2007122970, WO2007138485, WO2008006319, WO2008006969, WO2008010238, WO2008017398, WO2008028188, WO2008066356, WO2008084303, WO2008089461-WO2008089464, WO2008093639, WO2008096769, WO2008096820, WO2008096829, US2008194617, WO2008099944, WO2008108602, WO2008109334, WO2008126731, WO2008126732.

In one embodiment of the invention, the compound of the formula I is administered in combination with Competact™, a solid combination of pioglitazone hydrochloride with metformin hydrochloride.

In one embodiment of the invention, the compound of the formula I is administered in combination with Tandemact™, a solid combination of pioglitazone with glimepiride.

In a further embodiment of the invention, the compound of the formula I is administered in combination with a solid combination of pioglitazone hydrochloride with an angiotensin II agonist, for example TAK-536.

In one embodiment of the invention, the compound of the formula I is administered in combination with a PPAR alpha agonist or mixed PPAR alpha/PPAR delta agonist, for example GW9578, GW-590735, K-111, LY-674, KRP-101, DRF-10945, LY-518674, CP-900691, BMS-687453, BMS-711939, or those as described in WO2001040207, WO2002096894, WO2005097076, WO2007056771, WO2007087448, WO2007089667, WO2007089557, WO2007102515, WO2007103252, JP2007246474, WO2007118963, WO2007118964, WO2007126043, WO2008006043, WO2008006044, WO2008012470, WO2008035359, WO2008087365, WO2008087366, WO2008087367, WO2008117982.

In one embodiment of the invention, the compound of the formula I is administered in combination with a mixed PPAR alpha/gamma agonist, for example naveglitazar, LY-510929, ONO-5129, E-3030, AVE 8042, AVE 8134, AVE 0847, CKD-501 (lobeglitazone sulfate), MBX-213, KY-201 or as described in WO 00/64888, WO 00/64876, WO03/020269, WO2004024726, WO2007099553, US2007276041, WO2007085135, WO2007085136, WO2007141423, WO2008016175, WO2008053331, WO2008109697, WO2008109700, WO2008108735 or in J. P. Berger et al., TRENDS in Pharmacological Sciences 28(5), 244-251, 2005.

In one embodiment of the invention, the compound of the formula I is administered in combination with a PPAR delta agonist, for example GW-501516, or as described in WO2006059744, WO2006084176, WO2006029699, WO2007039172-WO2007039178, WO2007071766, WO2007101864, US2007244094, WO2007119887, WO2007141423, US2008004281, WO2008016175, WO2008066356, WO2008071311, WO2008084962, US2008176861.

In one embodiment of the invention, the compound of the formula I is administered in combination with a pan-SPPARM (selective PPAR modulator alpha, gamma, delta), for example GFT-505, or those as described in WO2008035359.

In one embodiment, the compound of the formula I is administered in combination with metaglidasen or with MBX-2044 or other partial PPAR gamma agonists/antagonists.

In one embodiment, the compound of the formula I is administered in combination with an a-glucosidase inhibitor, for example miglitol or acarbose, or those as described, for example, in WO2007114532, WO2007140230, US2007287674, US2008103201, WO2008065796, WO2008082017.

In one embodiment, the compound of the formula I is administered in combination with an inhibitor of glycogen phosphorylase, for example PSN-357 or FR-258900, or those as described in WO2003084922, WO2004007455, WO2005073229-31, WO2005067932, WO2008062739, WO2008099000, WO2008113760.

In one embodiment, the compound of the formula I is administered in combination with glucagon receptor antagonists, for example A-770077 or NNC-25-2504 or as described in WO2004100875, WO2005065680, WO2006086488, WO2007047177, WO2007106181, WO2007111864, WO2007120270, WO2007120284, WO2007123581, WO2007136577, WO2008042223, WO2008098244.

In a further embodiment, the compound of the formula I is administered in combination with an antisense compound, e.g. ISIS-325568, which inhibits the production of the glucagon receptor.

In one embodiment, the compound of the formula I is administered in combination with activators of glucokinase, for example LY-2121260 (WO2004063179), PSN-105, PSN-110, GKA-50, or those as described, for example, in WO2004072031, WO2004072066, WO2005080360, WO2005044801, WO2006016194, WO2006058923, WO2006112549, WO2006125972, WO2007017549, WO2007017649, WO2007007910, WO2007007040-42, WO2007006760-61, WO2007006814, WO2007007886, WO2007028135, WO2007031739, WO2007041365, WO2007041366, WO2007037534, WO2007043638, WO2007053345, WO2007051846, WO2007051845, WO2007053765, WO2007051847, WO2007061923, WO2007075847, WO2007089512, WO2007104034, WO2007117381, WO2007122482, WO2007125103, WO2007125105, US2007281942, WO2008005914, WO2008005964, WO2008043701, WO2008044777, WO2008047821, US2008096877, WO2008050117, WO2008050101, WO2008059625, US2008146625, WO2008078674, WO2008079787, WO2008084043, WO2008084044, WO2008084872, WO2008089892, WO2008091770, WO2008075073, WO2008084043, WO2008084044, WO2008084872, WO2008084873, WO2008089892, WO2008091770, JP2008189659, WO2008104994, WO2008111473, WO2008116107, WO2008118718, WO2008120754.

In one embodiment, the compound of the formula I is administered in combination with an inhibitor of gluconeogenesis, as described, for example, in FR-225654, WO2008053446.

In one embodiment, the compound of the formula I is administered in combination with inhibitors of fructose 1,6-bisphosphatase (FBPase), for example MB-07729, CS-917 (MB-06322) or MB-07803, or those as described in WO2006023515, WO2006104030, WO2007014619, WO2007137962, WO2008019309, WO2008037628.

In one embodiment, the compound of the formula I is administered in combination with modulators of glucose transporters 4 (GLUT4), for example KST-48 (D.-O. Lee et al.: Arzneim.-Forsch. Drug Res. 54 (12), 835 (2004)).

In one embodiment, the compound of the formula I is administered in combination with inhibitors of glutamine: fructose-6-phosphate amidotransferase (GFAT), as described, for example, in WO2004101528.

In one embodiment, the compound of the formula I is administered in combination with inhibitors of dipeptidyl peptidase IV (DPP-IV), for example vildagliptin (LAF-237), sitagliptin (MK-0431), sitagliptin phosphate, saxagliptin ((BMS-477118), GSK-823093, PSN-9301, SYR-322, SYR-619, TA-6666, TS-021, GRC-8200 (Melogliptin), GW-825964X, KRP-104, DP-893, ABT-341, ABT-279 or another salt thereof, S-40010, S-40755, PF-00734200, BI-1356, PHX-1149, alogliptin benzoate, linagliptin, melogliptin, or those compounds as described in WO2003074500, WO2003106456, WO2004037169, WO200450658, WO2005037828, WO2005058901, WO2005012312, WO2005/012308, WO2006039325, WO2006058064, WO2006015691, WO2006015701, WO2006015699, WO2006015700, WO2006018117, WO2006099943, WO2006099941, JP2006160733, WO2006071752, WO2006065826, WO2006078676, WO2006073167, WO2006068163, WO2006085685, WO2006090915, WO2006104356, WO2006127530, WO2006111261, US2006890898, US2006803357, US2006303661, WO2007015767 (LY-2463665), WO2007024993, WO2007029086, WO2007063928, WO2007070434, WO2007071738, WO2007071576, WO2007077508, WO2007087231, WO2007097931, WO2007099385, WO2007100374, WO2007112347, WO2007112669, WO2007113226, WO2007113634, WO2007115821, WO2007116092, US2007259900, EP1852108, US2007270492, WO2007126745, WO2007136603, WO2007142253, WO2007148185, WO2008017670, US2008051452, WO2008027273, WO2008028662, WO2008029217, JP2008031064, JP2008063256, WO2008033851, WO2008040974, WO2008040995, WO2008060488, WO2008064107, WO2008066070, WO2008077597, JP2008156318, WO2008087560, WO2008089636, WO2008093960, WO2008096841, WO2008101953, WO2008118848, WO2008119005, WO2008119208, WO2008120813, WO2008121506.

In one embodiment, the compound of the formula I is administered in combination with Janumet™, a solid combination of sitagliptin phosphate with metformin hydrochloride.

In one embodiment, the compound of the formula I is administered in combination with Eucreas®, a solid combination of vildagliptin with metformin hydrochloride.

In a further embodiment, the compound of the formula I is administered in combination with a solid combination of alogliptin benzoate with pioglitazone.

In one embodiment, the compound of the formula I is administered in combination with a solid combination of a salt of sitagliptin with metformin hydrochloride.

In one embodiment, the compound of the formula I is administered in combination with a combination of a DPP-IV inhibitor with omega-3 fatty acids or omega-3 fatty acid esters, as described, for example, in WO2007128801.

In one embodiment, the compound of the formula I is administered in combination with a solid combination of a salt of sitagliptin with metformin hydrochloride.

In one embodiment, the compound of the formula I is administered in combination with a substance which enhances insulin secretion, for example KCP-265 (WO2003097064), or those as described in WO2007026761, WO2008045484, US2008194617.

In one embodiment, the compound of the formula I is administered in combination with agonists of the glucose-dependent insulinotropic receptor (GDIR), for example APD-668.

In one embodiment of the invention, the compound of the formula I is administered in combination with an ATP citrate lyase inhibitor, for example SB-204990.

In one embodiment, the compound of the formula I is administered in combination with modulators of the sodium-dependent glucose transporter 1 or 2 (SGLT1, SGLT2), for example KGA-2727, T-1095, SGL-0010, AVE 2268, SAR 7226, SGL-5083, SGL-5085, SGL-5094, ISIS-388626, sergliflozin or dapagliflozin, or as described, for example, in WO2004007517, WO200452903, WO200452902, PCT/EP2005/005959, WO2005085237, JP2004359630, WO2005121161, WO2006018150, WO2006035796, WO2006062224, WO2006058597, WO2006073197, WO2006080577, WO2006087997, WO2006108842, WO2007000445, WO2007014895, WO2007080170, WO2007093610, WO2007126117, WO2007128480, WO2007129668, US2007275907, WO2007136116, WO2007143316, WO2007147478, WO2008001864, WO2008002824, WO2008013277, WO2008013280, WO2008013321, WO2008013322, WO2008016132, WO2008020011, JP2008031161, WO2008034859, WO2008042688, WO2008044762, WO2008046497, WO2008049923, WO2008055870, WO2008055940, WO2008069327, WO2008070609, WO2008071288, WO2008072726, WO2008083200, WO2008090209, WO2008090210, WO2008101586, WO2008101939, WO2008116179, WO2008116195, US2008242596 or by A. L. Handlon in Expert Opin. Ther. Patents (2005) 15(11), 1531-1540.

In one embodiment, the compound of the formula I is administered in combination with inhibitors of 11-beta-hydroxysteroid dehydrogenase 1 (11(3-HSD1), for example BVT-2733, JNJ-25918646, NCB-13739, INCB-20817, DIO-92 ((−)-ketoconazole) or those as described, for example, in WO200190090-94, WO200343999, WO2004112782, WO200344000, WO200344009, WO2004112779, WO2004113310, WO2004103980, WO2004112784, WO2003065983, WO2003104207, WO2003104208, WO2004106294, WO2004011410, WO2004033427, WO2004041264, WO2004037251, WO2004056744, WO2004058730, WO2004065351, WO2004089367, WO2004089380, WO2004089470-71, WO2004089896, WO2005016877, WO2005063247, WO2005097759, WO2006010546, WO2006012227, WO2006012173, WO2006017542, WO2006034804, WO2006040329, WO2006051662, WO2006048750, WO2006049952, WO2006048331, WO2006050908, WO2006024627, WO2006040329, WO2006066109, WO2006074244, WO2006078006, WO2006106423, WO2006132436, WO2006134481, WO2006134467, WO2006135795, WO2006136502, WO2006138508, WO2006138695, WO2006133926, WO2007003521, WO2007007688, US2007066584, WO2007029021, WO2007047625, WO2007051811, WO2007051810, WO2007057768, WO2007058346, WO2007061661, WO2007068330, WO2007070506, WO2007087150, WO2007092435, WO2007089683, WO2007101270, WO2007105753, WO2007107470, WO2007107550, WO2007111921, US2007207985, US2007208001, WO2007115935, WO2007118185, WO2007122411, WO2007124329, WO2007124337, WO2007124254, WO2007127688, WO2007127693, WO2007127704, WO2007127726, WO2007127763, WO2007127765, WO2007127901, US2007270424, JP2007291075, WO2007130898, WO2007135427, WO2007139992, WO2007144394, WO2007145834, WO2007145835, WO2007146761, WO2008000950, WO2008000951, WO2008003611, WO2008005910, WO2008006702, WO2008006703, WO2008011453, WO2008012532, WO2008024497, WO2008024892, WO2008032164, WO2008034032, WO2008043544, WO2008044656, WO2008046758, WO2008052638, WO2008053194, WO2008071169, WO2008074384, WO2008076336, WO2008076862, WO2008078725, WO2008087654, WO2008088540, WO2008099145, WO2008101885, WO2008101886, WO2008101907, WO2008101914, WO2008106128, WO2008110196, WO2008119017, WO2008120655, WO2008127924.

In one embodiment, the compound of the formula I is administered in combination with inhibitors of protein tyrosine phosphatase 1B (PTP-1B), as described, for example, in WO200119830-31, WO200117516, WO2004506446, WO2005012295, WO2005116003, WO2005116003, WO2006007959, DE 10 2004 060542.4, WO2007009911, WO2007028145, WO2007067612-615, WO2007081755, WO2007115058, US2008004325, WO2008033455, WO2008033931, WO2008033932, WO2008033934, WO2008089581.

In one embodiment of the invention, the compound of the formula I is administered in combination with an agonist of GPR109A (HM74A receptor agonists; NAR agonists (nicotinic acid receptor agonists)), for example nicotinic acid or “extended release niacin” in conjunction with MK-0524A (laropiprant) or MK-0524, or those compounds as described in WO2004041274, WO2006045565, WO2006045564, WO2006069242, WO2006085108, WO2006085112, WO2006085113, WO2006124490, WO2006113150, WO2007017261, WO2007017262, WO2007017265, WO2007015744, WO2007027532, WO2007092364, WO2007120575, WO2007134986, WO2007150025, WO2007150026, WO2008016968, WO2008051403, WO2008086949, WO2008091338, WO2008097535, WO2008099448, US2008234277, WO2008127591.

In another embodiment of the invention, the compound of the formula I is administered in combination with a solid combination of niacin with simvastatin.

In another embodiment of the invention, the compound of the formula I is administered in combination with nicotinic acid or “extended release niacin” in conjunction with MK-0524A (laropiprant).

In a further embodiment of the invention, the compound of the formula I is administered in combination with nicotinic acid or “extended release niacin” in conjunction with MK-0524A (laropiprant) and with simvastatin.

In one embodiment of the invention, the compound of the formula I is administered in combination with nicotinic acid or another nicotinic acid receptor agonist and a prostaglandin DP receptor antagonist, for example those as described in WO2008039882.

In another embodiment of the invention, the compound of the formula I is administered in combination with an agonist of GPR116, as described, for example, in WO2006067531, WO2006067532.

In one embodiment, the compound of the formula I is administered in combination with modulators of GPR40, as described, for example, in WO2007013689, WO2007033002, WO2007106469, US2007265332, WO2007123225, WO2007131619, WO2007131620, WO2007131621, US2007265332, WO2007131622, WO2007136572, WO2008001931, WO2008030520, WO2008030618, WO2008054674, WO2008054675, WO2008066097, US2008176912.

In one embodiment, the compound of the formula I is administered in combination with modulators of GPR119 (G-protein-coupled glucose-dependent insulinotropic receptor), for example PSN-119-1, PSN-821, PSN-119-2, MBX-2982 or those as described, for example, in WO2004065380, WO2005061489 (PSN-632408), WO2006083491, WO2007003960-62 and WO2007003964, WO2007035355, WO2007116229, WO2007116230, WO2008005569, WO2008005576, WO2008008887, WO2008008895, WO2008025798, WO2008025799, WO2008025800, WO2008070692, WO2008076243, WO200807692, WO2008081204, WO2008081205, WO2008081206, WO2008081207, WO2008081208, WO2008083238, WO2008085316, WO2008109702.

In a further embodiment, the compound of the formula I is administered in combination with modulators of GPR120, as described, for example, in EP1688138, WO2008066131, WO2008066131, WO2008103500, WO2008103501.

In one embodiment, the compound of the formula I is administered in combination with inhibitors of hormone-sensitive lipase (HSL) and/or phospholipases, as described, for example, in WO2005073199, WO2006074957, WO2006087309, WO2006111321, WO2007042178, WO2007119837, WO2008122352, WO2008122357.

In one embodiment, the compound of the formula I is administered in combination with inhibitors of endothelial lipase, as described, for example, in WO2007110216.

In one embodiment, the compound of the formula I is administered in combination with a phospholipase A2 inhibitor, for example darapladib or A-002, or those as described in WO2008048866, WO20080488867.

In one embodiment, the compound of the formula I is administered in combination with myricitrin, a lipase inhibitor (WO2007119827).

In one embodiment, the compound of the formula I is administered in combination with an inhibitor of glycogen synthase kinase-3 beta (GSK-3 beta), as described, for example, in US2005222220, WO2005085230, WO2005111018, WO2003078403, WO2004022544, WO2003106410, WO2005058908, US2005038023, WO2005009997, US2005026984, WO2005000836, WO2004106343, EP1460075, WO2004014910, WO2003076442, WO2005087727, WO2004046117, WO2007073117, WO2007083978, WO2007120102, WO2007122634, WO2007125109, WO2007125110, US2007281949, WO2008002244, WO2008002245, WO2008016123, WO2008023239, WO2008044700, WO2008056266, WO2008057940, WO2008077138, EP1939191, EP1939192, WO2008078196, WO2008094992, WO2008112642, WO2008112651, WO2008113469, WO2008121063, WO2008121064.

In one embodiment, the compound of the formula I is administered in combination with an inhibitor of phosphoenolpyruvate carboxykinase (PEPCK), for example those as described in WO2004074288.

In one embodiment, the compound of the formula I is administered in combination with an inhibitor of phosphoinositide kinase-3 (PI3K), for example those as described in WO2008027584, WO2008070150, WO2008125833, WO2008125835, WO2008125839.

In one embodiment, the compound of the formula I is administered in combination with an inhibitor of serum/glucocorticoid-regulated kinase (SGK), as described, for example, in WO2006072354, WO2007093264, WO2008009335, WO2008086854.

In one embodiment, the compound of the formula I is administered in combination with a modulator of the glucocorticoid receptor, as described, for example, in WO2008057855, WO2008057856, WO2008057857, WO2008057859, WO2008057862, WO2008059867, WO2008059866, WO2008059865, WO2008070507, WO2008124665, WO2008124745.

In one embodiment, the compound of the formula I is administered in combination with a modulator of the mineralocorticoid receptor (MR), for example drospirenone, or those as described in WO2008104306, WO2008119918.

In one embodiment, the compound of the formula I is administered in combination with an inhibitor of protein kinase C beta (PKC beta), for example ruboxistaurin, or those as described in WO2008096260, WO2008125945.

In one embodiment, the compound of the formula I is administered in combination with an inhibitor of protein kinase D, for example doxazosin (WO2008088006).

In a further embodiment, the compound of the formula I is administered in combination with an activator of the AMP-activated protein kinase (AMPK), as described, for example, in WO2007062568, WO2008006432, WO2008016278, WO2008016730, WO2008083124.

In one embodiment, the compound of the formula I is administered in combination with an inhibitor of ceramide kinase, as described, for example, in WO2007112914, WO2007149865.

In a further embodiment, the compound of the formula I is administered in combination with an inhibitor of MAPK-interacting kinase 1 or 2 (MNK1 or 2), as described, for example, in WO2007104053, WO2007115822, WO2008008547, WO2008075741.

In one embodiment, the compound of the formula I is administered in combination with inhibitors of “I-kappaB kinase” (IKK inhibitors), as described, for example, in WO2001000610, WO2001030774, WO2004022057, WO2004022553, WO2005097129, WO2005113544, US2007244140, WO2008099072, WO2008099073, WO2008099073, WO2008099074, WO2008099075.

In another embodiment, the compound of the formula I is administered in combination with inhibitors of NF-kappaB (NFKB) activation, for example salsalate.

In a further embodiment, the compound of the formula I is administered in combination with inhibitors of ASK-1 (apoptosis signal-regulating kinase 1), as described, for example, in WO2008016131.

In one embodiment of the invention, the compound of the formula I is administered in combination with an HMG-CoA reductase inhibitor such as simvastatin, fluvastatin, pravastatin, lovastatin, atorvastatin, cerivastatin, rosuvastatin, pitavastatin, L-659699, BMS-644950, or those as described in US2007249583, WO2008083551.

In a further embodiment of the invention, the compound of the formula I is administered in combination with a farnesoid X receptor (FXR) modulator, for example WAY-362450 or those as described in WO2003099821, WO2005056554, WO2007052843, WO2007070796, WO2007092751, JP2007230909, WO2007095174, WO2007140174, WO2007140183, WO2008000643, WO2008002573, WO2008025539, WO2008025540, JP2008214222.

In another embodiment of the invention, the compound of the formula I is administered in combination with a ligand of the liver X receptor (LXR), as described, for example, in WO2007092965, WO2008041003, WO2008049047, WO2008065754, WO2008073825, US2008242677.

In one embodiment of the invention, the compound of the formula I is administered in combination with a fibrate, for example fenofibrate, clofibrate, bezafibrate, or those as described in WO2008093655.

In one embodiment of the invention, the compound of the formula I is administered in combination with fibrates, for example the choline salt of fenofibrate (SLV-348).

In one embodiment of the invention, the compound of the formula I is administered in combination with fibrates, for example the choline salt of fenofibrate and an HMG-CoA reductase inhibitor, for example rosuvastatin.

In a further embodiment of the invention, the compound of the formula I is administered in combination with bezafibrate and diflunisal.

In a further embodiment of the invention, the compound of the formula I is administered in combination with a solid combination of fenofibrate or a salt thereof with simvastatin, rosuvastatin, fluvastatin, lovastatin, cerivastatin, pravastatin, pitavastatin or atorvastatin.

In a further embodiment of the invention, the compound of the formula I is administered in combination with Synordia (R), a solid combination of fenofibrate with metformin.

In one embodiment of the invention, the compound of the formula I is administered in combination with a cholesterol reabsorption inhibitor, for example ezetimibe, tiqueside, pamaqueside, FM-VP4 (sitostanol/campesterol ascorbyl phosphate; Forbes Medi-Tech, WO2005042692, WO2005005453), MD-0727 (Microbia Inc., WO2005021497, WO2005021495) or with compounds as described in WO2002066464, WO2005000353 (Kotobuki Pharmaceutical Co. Ltd.) or WO2005044256 or WO2005062824 (Merck & Co.) or WO2005061451 and WO2005061452 (AstraZeneca AB) and WO2006017257 (Phenomix) or WO2005033100 (Lipideon Biotechnology AG), or as described in WO2002050060, WO2002050068, WO2004000803, WO2004000804, WO2004000805, WO2004087655, WO2004097655, WO2005047248, WO2006086562, WO2006102674, WO2006116499, WO2006121861, WO2006122186, WO2006122216, WO2006127893, WO2006137794, WO2006137796, WO2006137782, WO2006137793, WO2006137797, WO2006137795, WO2006137792, WO2006138163, WO2007059871, US2007232688, WO2007126358, WO2008033431, WO2008033465, WO2008052658, WO2008057336, WO2008085300.

In one embodiment of the invention, the compound of the formula I is administered in combination with an NPC1L1 antagonist, for example those as described in WO2008033464, WO2008033465.

In one embodiment of the invention, the compound of the formula I is administered in combination with Vytorin™, a solid combination of ezetimibe with simvastatin.

In one embodiment of the invention, the compound of the formula I is administered in combination with a solid combination of ezetimibe with atorvastatin.

In one embodiment of the invention, the compound of the formula I is administered in combination with a solid combination of ezetimibe with fenofibrate.

In one embodiment of the invention, the further active ingredient is a diphenylazetidinone derivative, as described, for example, in U.S. Pat. No. 6,992,067 or U.S. Pat. No. 7,205,290.

In a further embodiment of the invention, the further active ingredient is a diphenylazetidinone derivative, as described, for example, in U.S. Pat. No. 6,992,067 or U.S. Pat. No. 7,205,290, combined with a statin, for example simvastatin, fluvastatin, pravastatin, lovastatin, cerivastatin, atorvastatin, pitavastatin or rosuvastatin.

In one embodiment of the invention, the compound of the formula I is administered in combination with a solid combination of lapaquistat, a squalene synthase inhibitor, with atorvastatin.

In one embodiment of the invention, the compound of the formula I is administered in combination with a CETP inhibitor, for example torcetrapib, anacetrapib or JTT-705 (dalcetrapib), or those as described in WO2006002342, WO2006010422, WO2006012093, WO2006073973, WO2006072362, WO2007088996, WO2007088999, US2007185058, US2007185113, US2007185154, US2007185182, WO2006097169, WO2007041494, WO2007090752, WO2007107243, WO2007120621, US2007265252, US2007265304, WO2007128568, WO2007132906, WO2008006257, WO2008009435, WO2008018529, WO2008058961, WO2008058967, WO2008059513, WO2008070496, WO2008115442, WO2008111604.

In one embodiment of the invention, the compound of the formula I is administered in combination with bile acid reabsorption inhibitors (inhibitors of the intestinal bile acid transporter (IBAT)) (see, for example, U.S. Pat. No. 6,245,744, U.S. Pat. No. 6,221,897 or WO00/61568), for example HMR 1741, or those as described in DE 10 2005 033099.1 and DE 10 2005 033100.9, DE 10 2006 053635, DE 10 2006 053637, WO2007009655-56, WO2008058628, WO2008058629, WO2008058630, WO2008058631.

In one embodiment, the compound of the formula I is administered in combination with agonists of GPBAR1 (G-protein-coupled bile acid receptor-1; TGR5), as described, for example, in US20060199795, WO2007110237, WO2007127505, WO2008009407, WO2008067219, WO2008067222, FR2908310, WO2008091540, WO2008097976.

In one embodiment of the invention, the compound of the formula I is administered in combination with inhibitors of the TRPM5 channel (TRP cation channel M5), as described, for example, in WO2008097504.

In one embodiment of the invention, the compound of the formula I is administered in combination with a polymeric bile acid adsorber, for example cholestyramine, colesevelam hydrochloride.

In one embodiment of the invention, the compound of the formula I is administered in combination with colesevelam hydrochloride and metformin or a sulfonylurea or insulin.

In one embodiment of the invention, the compound of the formula I is administered in combination with a chewing gum comprising phytosterols (Reductol™)

In one embodiment of the invention, the compound of the formula I is administered in combination with an inhibitor of the microsomal triglyceride transfer protein (MTP inhibitor), for example implitapide, BMS-201038, R-103757, AS-1552133, SLx-4090, AEGR-733, or those as described in WO2005085226, WO2005121091, WO2006010423, WO2006113910, WO2007143164, WO2008049806, WO2008049808, WO2008090198, WO2008100423.

In a further embodiment of the invention, the compound of the formula I is administered in combination with a combination of a cholesterol absorption inhibitor, for example ezetimibe, and an inhibitor of the triglyceride transfer protein (MTP inhibitor), for example implitapide, as described in WO2008030382 or in WO2008079398.

In one embodiment of the invention, the compound of the formula I is administered in combination with an active antihypertriglyceridemic ingredient, for example those as described in WO2008032980.

In another embodiment of the invention, the compound of the formula I is administered in combination with an antagonist of the somatostatin 5 receptor (SSTS receptor), for example those as described in WO2006094682.

In one embodiment of the invention, the compound of the formula I is administered in combination with an ACAT inhibitor, for example avasimibe, SMP-797 or KY-382, or those as described in WO2008087029, WO2008087030, WO2008095189.

In a further embodiment of the invention, the compound of the formula I is administered in combination with an inhibitor of liver carnitine palmitoyltransferase 1 (L-CPT1), as described, for example, in WO2007063012, WO2007096251 (ST-3473), WO2008015081, US2008103182, WO2008074692.

In a further embodiment of the invention, the compound of the formula I is administered in combination with a modulator of serine palmitoyltransferase (SPT), as described, for example, in WO2008031032, WO2008046071, WO2008083280, WO2008084300.

In one embodiment of the invention, the compound of the formula I is administered in combination with a squalene synthetase inhibitor, for example BMS-188494, TAK-475 (lapaquistat acetate), or as described in WO2005077907, JP2007022943, WO2008003424.

In one embodiment of the invention, the compound of the formula I is administered in combination with ISIS-301012 (mipomersen), an antisense oligonucleotide which is capable of regulating the apolipoprotein B gene.

In one embodiment of the invention, the compound of the formula I is administered in combination with a stimulator of the ApoA-1 gene, as described, for example in WO2008092231.

In one embodiment of the invention, the compound of the formula I is administered in combination with an LDL receptor inducer (see U.S. Pat. No. 6,342,512), for example HMR1171, HMR1586, or those as described in WO2005097738, WO2008020607.

In another embodiment of the invention, the compound of the formula I is administered in combination with an HDL cholesterol-elevating agent, for example those as described in WO2008040651, WO2008099278.

In one embodiment of the invention, the compound of the formula I is administered in combination with an ABCA1 expression enhancer, as described, for example, in WO2006072393, WO2008062830.

In one embodiment of the invention, the compound of the formula I is administered in combination with a lipoprotein lipase modulator, for example ibrolipim (NO-1886).

In one embodiment of the invention, the compound of the formula I is administered in combination with a lipoprotein(a) antagonist, for example gemcabene (CI-1027).

In one embodiment of the invention, the compound of the formula I is administered in combination with a lipase inhibitor, for example orlistat or cetilistat (ATL-962).

In one embodiment of the invention, the compound of the formula I is administered in combination with an adenosine A1 receptor agonist (adenosine A1 R), as described, for example, in EP1258247, EP1375508, WO2008028590, WO2008077050.

In one embodiment of the invention, the compound of the formula I is administered in combination with an adenosine A2B receptor agonist (adenosine A2B R), for example ATL-801.

In another embodiment of the invention, the compound of the formula I is administered in combination with a modulator of adenosine A2A and/or adenosine A3 receptors, as described, for example, in WO2007111954, WO2007121918, WO2007121921, WO2007121923, WO2008070661.

In a further embodiment of the invention, the compound of the formula I is administered in combination with an agonist of the adenosine A1/A2B receptors, as described, for example, in WO2008064788, WO2008064789.

In one embodiment of the invention, the compound of the formula I is administered in combination with an adenosine A2B receptor antagonist (adenosine A2B R), as described in US2007270433, WO2008027585, WO2008080461.

In one embodiment, the compound of the formula I is administered in combination with inhibitors of acetyl-CoA carboxylase (ACC1 and/or ACC2), for example those as described in WO199946262, WO200372197, WO2003072197, WO2005044814, WO2005108370, JP2006131559, WO2007011809, WO2007011811, WO2007013691, WO2007095601-603, WO2007119833, WO2008065508, WO2008069500, WO2008070609, WO2008072850, WO2008079610, WO2008088688, WO2008088689, WO2008088692, US2008171761, WO2008090944, JP2008179621, US2008200461, WO2008102749, WO2008103382, WO2008121592.

In another embodiment, the compound of the formula I is administered in combination with modulators of microsomal acyl-CoA:glycerol-3-phosphate acyltransferase 3 (GPAT3, described in WO2007100789) or with modulators of microsomal acyl-CoA:glycerol-3-phosphate acyltransferase 4 (GPAT4, described in WO2007100833).

In a further embodiment, the compound of the formula I is administered in combination with modulators of xanthine oxidoreductase (XOR).

In another embodiment, the compound of the formula I is administered in combination with inhibitors of soluble epoxide hydrolase (sEH), as described, for example, in WO2008051873, WO2008051875, WO2008073623, WO2008094869, WO2008112022.

In a further embodiment, the compound of the formula I is administered in combination with CART modulators (see “Cocaine-amphetamine-regulated transcript influences energy metabolism, anxiety and gastric emptying in mice” Asakawa, A. et al.: Hormone and Metabolic Research (2001), 33(9), 554-558);

NPY antagonists, for example N-{4-[(4-aminoquinazolin-2-ylamino)methyl]-cyclohexylmethyl}naphthalene-1-sulfonamide hydrochloride (CGP 71683A) or velneperit;
NPY-5 receptor antagonists, such as L-152804 or the compound “NPY-5-BY” from Banyu, or as described, for example, in WO2006001318, WO2007103295, WO2007125952, WO2008026563, WO2008026564, WO2008052769, WO2008092887, WO2008092888, WO2008092891;
NPY-4 receptor antagonists, as described, for example, in WO2007038942;
NPY-2 receptor antagonists, as described, for example, in WO2007038943;
peptide YY 3-36 (PYY3-36) or analogous compounds, for example CJC-1682 (PYY3-36 conjugated with human serum albumin via Cys34) or CJC-1643 (derivative of PYY3-36, which is conjugated in vivo to serum albumin), or those as described in WO2005080424, WO2006095166, WO2008003947;
derivatives of the peptide obestatin, as described by WO2006096847;
CB1R (cannabinoid receptor 1) antagonists, for example rimonabant, surinabant (SR147778), SLV-319 (ibipinabant), AVE-1625, taranabant (MK-0364) or salts thereof, otenabant (CP-945,598), rosonabant, V-24343 or those compounds as described in, for example, EP 0656354, WO 00/15609, WO2001/64632-64634, WO 02/076949, WO2005080345, WO2005080328, WO2005080343, WO2005075450, WO2005080357, WO200170700, WO2003026647-48, WO200302776, WO2003040107, WO2003007887, WO2003027069, U.S. Pat. No. 6,509,367, WO200132663, WO2003086288, WO2003087037, WO2004048317, WO2004058145, WO2003084930, WO2003084943, WO2004058744, WO2004013120, WO2004029204, WO2004035566, WO2004058249, WO2004058255, WO2004058727, WO2004069838, US20040214837, US20040214855, US20040214856, WO2004096209, WO2004096763, WO2004096794, WO2005000809, WO2004099157, US20040266845, WO2004110453, WO2004108728, WO2004000817, WO2005000820, US20050009870, WO200500974, WO2004111033-34, WO200411038-39, WO2005016286, WO2005007111, WO2005007628, US20050054679, WO2005027837, WO2005028456, WO2005063761-62, WO2005061509, WO2005077897, WO2006018662, WO2006047516, WO2006060461, WO2006067428, WO2006067443, WO2006087480, WO2006087476, WO2006100208, WO2006106054, WO2006111849, WO2006113704, WO2007009705, WO2007017124, WO2007017126, WO2007018459, WO2007018460, WO2007016460, WO2007020502, WO2007026215, WO2007028849, WO2007031720, WO2007031721, WO2007036945, WO2007038045, WO2007039740, US20070015810, WO2007046548, WO2007047737, WO2007057687, WO2007062193, WO2007064272, WO2007079681, WO2007084319, WO2007084450, WO2007086080, EP1816125, US2007213302, WO2007095513, WO2007096764, US2007254863, WO2007119001, WO2007120454, WO2007121687, WO2007123949, US2007259934, WO2007131219, WO2007133820, WO2007136571, WO2007136607, WO2007136571, U.S. Pat. No. 7,297,710, WO2007138050, WO2007139464, WO2007140385, WO2007140439, WO2007146761, WO2007148061, WO2007148062, US2007293509, WO2008004698, WO2008017381, US2008021031, WO2008024284, WO2008031734, WO2008032164, WO2008034032, WO2008035356, WO2008036021, WO2008036022, WO2008039023, WO2998043544, WO2008044111, WO2008048648, EP1921072-A1, WO2008053341, WO2008056377, WO2008059207, WO2008059335, WO2008062424, WO2008068423, WO2008068424, WO2008070305, WO2008070306, WO2008074816, WO2008074982, WO2008075012, WO2008075013, WO2008075019, WO2008075118, WO2008076754, WO2008081009, WO2008084057, EP1944295, US2008090809, US2008090810, WO2008092816, WO2008094473, WO2008094476, WO2008099076, WO2008099139, WO2008101995, US2008207704, WO2008107179, WO2008109027, WO2008112674, WO2008115705, WO2008118414, WO2008119999, WO200812000, WO2008121257, WO2008127585;
cannabinoid receptor 1/cannabinoid receptor 2 (CB1/CB2) modulating compounds, for example delta-9-tetrahydrocannabivarin, or those as described, for example, in WO2007001939, WO2007044215, WO2007047737, WO2007095513, WO2007096764, WO2007112399, WO2007112402, WO2008122618;
modulators of FAAH (fatty acid amide hydrolase), as described, for example, in WO2007140005, WO2008019357, WO2008021625, WO2008023720, WO2008030532;
inhibitors of fatty acid synthase (FAS), as described, for example, in WO2008057585, WO2008059214, WO2008075064, WO2008075070, WO2008075077;
inhibitors of LCE (long chain fatty acid elongase), as described, for example, in WO2008120653;
vanilloid-1 receptor modulators (modulators of TRPV1), as described, for example, in WO2007091948, WO2007129188, WO2007133637, WO2008007780, WO2008010061, WO2008007211, WO2008010061, WO2008015335, WO2008018827, WO2008024433, WO2008024438, WO2008032204, WO2008050199, WO2008059339, WO2008059370, WO2008066664, WO2008075150, WO2008090382, WO2008090434, WO2008093024, WO2008107543, WO2008107544, WO2008110863;
modulators, antagonists or inverse agonists of the opioid receptors, for example GSK-982 or those as described, for example, in WO2007047397, WO2008021849, WO2008021851, WO2008032156, WO2008059335;
modulators of the “orphan opioid (ORL-1) receptor”, as described, for example, in US2008249122, WO2008089201;
agonists of the prostaglandin receptor, for example bimatoprost or those compounds as described in WO2007111806;
MC4 receptor agonists (melanocortin-4 receptor agonists, MC4R agonists, for example N-[2-(3a-benzyl-2-methyl-3-oxo-2,3,3a,4,6,7-hexahydropyrazolo[4,3-c]pyridin-5-yl)-1-(4-chlorophenyl)-2-oxoethyl]-1-amino-1,2,3,4-tetrahydronaphthalene-2-carboxamide; (WO 01/91752)) or LB53280, LB53279, LB53278 or THIQ, MB243, RY764, CHIR-785, PT-141, MK-0493, or those as described in WO2005060985, WO2005009950, WO2004087159, WO2004078717, WO2004078716, WO2004024720, US20050124652, WO2005051391, WO2004112793, WOUS20050222014, US20050176728, US20050164914, US20050124636, US20050130988, US20040167201, WO2004005324, WO2004037797, WO2005042516, WO2005040109, WO2005030797, US20040224901, WO200501921, WO200509184, WO2005000339, EP1460069, WO2005047253, WO2005047251, WO2005118573, EP1538159, WO2004072076, WO2004072077, WO2006021655-57, WO2007009894, WO2007015162, WO2007041061, WO2007041052, JP2007131570, EP-1842846, WO2007096186, WO2007096763, WO2007141343, WO2008007930, WO2008017852, WO2008039418, WO2008087186, WO2008087187, WO2008087189, WO2008087186-WO2008087190, WO2008090357;
orexin receptor 1 antagonists (OX1R antagonists), orexin receptor 2 antagonists (OX2R antagonists) or mixed OX1R/OX2R antagonists (e.g. 1-(2-methylbenzoxazol-6-yl)-3-[1,5]naphthyridin-4-ylurea hydrochloride (SB-334867-A), or those as described, for example, in WO200196302, WO200185693, WO2004085403, WO2005075458, WO2006067224, WO2007085718, WO2007088276, WO2007116374; WO2007122591, WO2007126934, WO2007126935, WO2008008517, WO2008008518, WO2008008551, WO2008020405, WO2008026149, WO2008038251, US2008132490, WO2008065626, WO2008078291, WO2008087611, WO2008081399, WO2008108991, WO2008107335, US2008249125);
histamine H3 receptor antagonists/inverse agonists (e.g. 3-cyclohexyl-1-(4,4-dimethyl-1,4,6,7-tetrahydroimidazo[4,5-c]pyridin-5-yl)propan-1-one oxalic acid salt (WO 00/63208), or those as described in WO200064884, WO2005082893, US2005171181 (e.g. PF-00389027), WO2006107661, WO2007003804, WO2007016496, WO2007020213, WO2007049798, WO2007055418, WO2007057329, WO2007065820, WO2007068620, WO2007068641, WO2007075629, WO2007080140, WO2007082840, WO2007088450, WO2007088462, WO2007094962, WO2007099423, WO2007100990, WO2007105053, WO2007106349, WO2007110364, WO2007115938, WO2007131907, WO2007133561, US2007270440, WO2007135111, WO2007137955, US2007281923, WO2007137968, WO2007138431, WO2007146122, WO2008005338, WO2008012010, WO2008015125, WO2008045371, EP1757594, WO2008068173, WO2008068174, US20080171753, WO2008072703, WO2008072724, US2008188484, US2008188486, US2008188487, WO2008109333, WO2008109336);
histamine H1/histamine H3 modulators, for example betahistine or its dihydrochloride;
modulators of the histamine H3 transporter or of the histamine H3/serotonin transporter, as described, for example, in WO2008002816, WO2008002817, WO2008002818, WO2008002820;
histamine H4 modulators, as described, for example, in WO2007117399;
CRF antagonists (e.g. [2-methyl-9-(2,4,6-trimethylphenyl)-9H-1,3,9-triazafluoren-4-yl]dipropylamine (WO 00/66585) or those CRF1 antagonists as described in WO2007105113, WO2007133756, WO2008036541, WO2008036579, WO2008083070);
CRF BP antagonists (e.g. urocortin);
urocortin agonists;
modulators of the beta-3 adrenoceptor, for example 1-(4-chloro-3-methanesulfonylmethylphenyl)-2-[2-(2,3-dimethyl-1H-indo1-6-yloxy)ethylamino]ethanol hydrochloride (WO 01/83451) or solabegron (GW-427353) or N-5984 (KRP-204), or those as described in JP2006111553, WO2002038543, WO2002038544, WO2007048840-843, WO2008015558, EP1947103;
MSH (melanocyte-stimulating hormone) agonists;
MCH (melanine-concentrating hormone) receptor antagonists (for example NBI-845, A-761, A-665798, A-798, ATC-0175, T-226296, T-71 (AMG-071, AMG-076), GW-856464, NGD-4715, ATC-0453, ATC-0759, GW-803430, or those compounds as described in WO2005085200, WO2005019240, WO2004011438, WO2004012648, WO2003015769, WO2004072025, WO2005070898, WO2005070925, WO2004039780, WO2004092181, WO2003033476, WO2002006245, WO2002089729, WO2002002744, WO2003004027, FR2868780, WO2006010446, WO2006038680, WO2006044293, WO2006044174, JP2006176443, WO2006018280, WO2006018279, WO2006118320, WO2006130075, WO2007018248, WO2007012661, WO2007029847, WO2007024004, WO2007039462, WO2007042660, WO2007042668, WO2007042669, US2007093508, US2007093509, WO2007048802, JP2007091649, WO2007092416; WO2007093363-366, WO2007114902, WO2007114916, WO2007141200, WO2007142217, US2007299062, WO2007146758, WO2007146759, WO2008001160, WO2008016811, WO2008020799, WO2008022979, WO2008038692, WO2008041090, WO2008044632, WO2008047544, WO2008061109, WO2008065021, WO2008068265, WO2008071646, WO2008076562, JP2008088120, WO2008086404, WO2008086409);
CCK-A (CCK-1) agonists/modulators (for example {2-[4-(4-chloro-2,5-dimethoxyphenyl)-5-(2-cyclohexylethyl)thiazol-2-ylcarbamoyl]-5,7-dimethylindol-1-yl}acetic acid trifluoroacetic acid salt (WO 99/15525) or SR-146131 (WO 0244150) or SSR-125180), or those as described in WO2005116034, WO2007120655, WO2007120688, WO2007120718, WO2008091631;
serotonin reuptake inhibitors (e.g. dexfenfluramine), or those as described in WO2007148341, WO2008034142, WO2008081477, WO2008120761;
mixed serotonin/dopamine reuptake inhibitors (e.g. bupropion), or those as described in WO2008063673, or solid combinations of bupropion with naltrexone or bupropion with zonisamide;
mixed reuptake inhibitors, for example DOV-21947;
mixed serotoninergic and noradrenergic compounds (e.g. WO 00/71549);
5-HT receptor agonists, for example 1-(3-ethylbenzofuran-7-yl)piperazine oxalic acid salt (WO 01/09111);
mixed dopamine/norepinephrine/acetylcholine reuptake inhibitors (e.g. tesofensine), or those as described, for example, in WO2006085118;
dopamine antagonists, as described, for example, in WO2008079838, WO2008079839, WO2008079847, WO2008079848;
norepinephrine reuptake inhibitors, as described, for example, in US2008076724;
5-HT2A receptor antagonists, as described, for example, in WO2007138343;
5-HT2C receptor agonists (for example lorcaserine hydrochloride (APD-356) or BVT-933, or those as described in WO200077010, WO200077001-02, WO2005019180, WO2003064423, WO200242304, WO2005035533, WO2005082859, WO2006004937, US2006025601, WO2006028961, WO2006077025, WO2006103511, WO2007028132, WO2007084622, US2007249709; WO2007132841, WO2007140213, WO2008007661, WO2008007664, WO2008009125, WO2008010073, WO2008108445);
5-HT6 receptor modulators, for example E-6837, BVT-74316 or PRX-07034, or those as described, for example, in WO2005058858, WO2007054257, WO2007107373, WO2007108569, WO2007108742-744, WO2008003703, WO2008027073, WO2008034815, WO2008054288, EP1947085, WO2008084491, WO2008084492, WO2008092665, WO2008092666, WO2008101247, WO2008110598, WO2008116831, WO2008116833;
agonists of estrogen receptor gamma (ERRγ agonists), as described, for example, in WO2007131005, WO2008052709;
agonists of estrogen receptor alpha (ERRα/ERR1 agonists), as described, for example, in WO2008109727;
sigma-1 receptor antagonists, as described, for example, in WO2007098953, WO2007098961, WO2008015266, WO2008055932, WO2008055933;
muscarin 3 receptor (M3R) antagonists, as described, for example, in WO2007110782, WO2008041184;
bombesin receptor agonists (BRS-3 agonists), as described, for example, in WO2008051404, WO2008051405, WO2008051406, WO2008073311;
galanin receptor antagonists;
growth hormone (e.g. human growth hormone or AOD-9604);
growth hormone releasing compounds (tert-butyl 6-benzyloxy-1-(2-diisopropylaminoethylcarbamoyl)-3,4-dihydro-1H-isoquinoline-2-carboxylate (WO 01/85695));
growth hormone secretagogue receptor antagonists (ghrelin antagonists), for example A-778193, or those as described in WO2005030734, WO2007127457, WO2008008286;
growth hormone secretagogue receptor modulators (ghrelin modulators), for example JMV-2959, JMV-3002, JMV-2810, JMV-2951, or those as described in WO2006012577 (e.g. YIL-781 or YIL-870), WO2007079239, WO2008092681;
TRH agonists (see, for example, EP 0 462 884);
decoupling protein 2 or 3 modulators;
chemical decouplers (e.g. WO2008059023, WO2008059024, WO2008059025, WO2008059026);
leptin agonists (see, for example, Lee, Daniel W.; Leinung, Matthew C.; Rozhayskaya-Arena, Marina; Grasso, Patricia. Leptin agonists as a potential approach to the treatment of obesity. Drugs of the Future (2001), 26(9), 873-881);
DA agonists (bromocriptin, doprexin);
lipase/amylase inhibitors (e.g. WO 00/40569, WO2008107184);
inhibitors of diacylglycerol O-acyltransferases (DGATs), for example BAY-74-4113, or as described, for example, in US2004/0224997, WO2004094618, WO200058491, WO2005044250, WO2005072740, JP2005206492, WO2005013907, WO2006004200, WO2006019020, WO2006064189, WO2006082952, WO2006120125, WO2006113919, WO2006134317, WO2007016538, WO2007060140, JP2007131584, WO2007071966, WO2007126957, WO2007137103, WO2007137107, WO2007138304, WO2007138311, WO2007141502, WO2007141517, WO2007141538, WO2007141545, WO2007144571, WO2008011130, WO2008011131, WO2008039007, WO2008048991, WO2008067257, WO2008099221;
inhibitors of monoacylglycerol acyltransferase (2-acylglycerol O-acyltransferase; MGAT), as described, for example, in WO2008038768;
inhibitors of fatty acid synthase (FAS), for example C75, or those as described in WO2004005277, WO2008006113;
inhibitors of stearoyl-CoA delta9 desaturase (SCD1), as described, for example, in WO2007009236, WO2007044085, WO2007046867, WO2007046868, WO20070501124, WO2007056846, WO2007071023, WO2007130075, WO2007134457, WO2007136746, WO2007143597, WO2007143823, WO2007143824, WO2008003753, WO2008017161, WO2008024390, WO2008029266, WO2008036715, WO2008043087, WO2008044767, WO2008046226, WO2008056687, WO2008062276, WO2008064474, WO2008074824, WO2008074832, WO2008074833, WO2008074834, WO2008074835, WO2008089580, WO2008096746, WO2008104524, WO2008116898, US2008249100, WO2008120744, WO2008120759, WO2008123469, WO2008127349;
inhibitors of fatty acid desaturase 1 (deltas desaturase), as described, for example, in WO2008089310;
hypoglycemic/hypertriglyceridemic indoline compounds, as described in WO2008039087;
inhibitors of “adipocyte fatty acid-binding protein aP2”, for example BMS-309403;
activators of adiponectin secretion, as described, for example, in WO2006082978, WO2008105533;
promoters of adiponectin secretion, as described, for example, in WO2007125946, WO2008038712;
modified adiponectins, as described, for example, in WO2008121009;
oxyntomodulin or analogs thereof;
oleoyl-estrone
or agonists or partial agonists of the thyroid hormone receptor (thyroid hormone receptor agonists), for example: KB-2115 (eprotirome), QRX-431 (sobetirome) or DITPA, or those as described in WO20058279, WO200172692, WO200194293, WO2003084915, WO2004018421, WO2005092316, WO2007003419, WO2007009913, WO2007039125, WO2007110225, WO2007110226, WO2007128492, WO2007132475, WO2007134864, WO2008001959, WO2008106213;
or agonists of the thyroid hormone receptor beta (TR-beta), for example MB-07811 or MB-07344, or those as described in WO2008062469.

In one embodiment of the invention, the compound of the formula I is administered in combination with a combination of eprotirome with ezetimibe.

In one embodiment of the invention, the compound of the formula I is administered in combination with an inhibitor of site-1 protease (S1P), for example PF-429242.

In a further embodiment of the invention, the compound of the formula I is administered in combination with a modulator of the “trace amine associated receptor 1” (TAAR1), as described, for example, in US2008146523, WO2008092785.

In one embodiment of the invention, the compound of the formula I is administered in combination with an inhibitor of growth factor receptor bound protein 2 (GRB2), as described, for example, in WO2008067270.

In a further embodiment of the invention, the compound of the formula I is administered in combination with an RNAi (siRNA) therapeutic agent directed against PCSK9 (proprotein convertase subtilisinikexin type 9).

In one embodiment, the compound of the formula I is administered in combination with Omacor® or Lovaza™ (omega-3 fatty acid ester; highly concentrated ethyl ester of eicosapentaenoic acid and of docosahexaenoic acid).

In one embodiment, the compound of the formula I is administered in combination with lycopene.

In one embodiment of the invention, the compound of the formula I is administered in combination with an antioxidant, for example OPC-14117, AGI-1067 (succinobucol), probucol, tocopherol, ascorbic acid, β-carotene or selenium.

In one embodiment of the invention, the compound of the formula I is administered in combination with a vitamin, for example vitamin B6 or vitamin B12.

In one embodiment, the compound of the formula I is administered in combination with more than one of the aforementioned compounds, for example in combination with a sulfonylurea and metformin, a sulfonylurea and acarbose, repaglinide and metformin (PrandiMet™), insulin and a sulfonylurea, insulin and metformin, insulin and troglitazone, insulin and lovastatin, etc.

In another embodiment, the compound of the formula I is administered in combination with an inhibitor of carboanhydrase type 2 (carbonic anhydrase type 2), for example those as described in WO2007065948.

In another embodiment, the compound of the formula I is administered in combination with topiramat or a derivative thereof, as described in WO2008027557.

In a further embodiment, the compound of the formula I is administered in combination with a solid combination of topiramat with phentermin (Qnexa™)

In a further embodiment, the compound of the formula I is administered in combination with an antisense compound, e.g. ISIS-377131, which inhibits the production of the glucocorticoid receptor.

In another embodiment, the compound of the formula I is administered in combination with an aldosterone synthase inhibitor and an antagonist of the glucocorticoid receptor, a cortisol synthesis inhibitor and/or an antagonist of the corticotropin releasing factor, as described, for example, in EP1886695, WO2008119744.

In one embodiment, the compound of the formula I is administered in combination with an agonist of the RUP3 receptor, as described, for example, in WO2007035355, WO2008005576.

In another embodiment, the compound of the formula I is administered in combination with an activator of the gene which codes for ataxia telangiectasia mutated (ATM) protein kinase, for example chloroquine.

In one embodiment, the compound of the formula I is administered in combination with a tau protein kinase 1 inhibitor (TPK1 inhibitor), as described, for example, in WO2007119463.

In one embodiment, the compound of the formula I is administered in combination with a “c-Jun N-terminal kinase” inhibitor (JNK inhibitor), as described, for example, in WO2007125405, WO2008028860, WO2008118626.

In one embodiment, the compound of the formula I is administered in combination with an endothelin A receptor antagonist, for example avosentan (SPP-301).

In one embodiment, the compound of the formula I is administered in combination with modulators of the glucocorticoid receptor (GR), for example KB-3305 or those compounds as described, for example, in WO2005090336, WO2006071609, WO2006135826, WO2007105766, WO2008120661.

In one embodiment, the further active ingredient is varenicline tartrate, a partial agonist of the alpha 4-beta 2 nicotinic acetylcholine receptor.

In one embodiment, the further active ingredient is trodusquemine.

In one embodiment, the further active ingredient is a modulator of the enzyme SIRT1 and/or SIRT3 (an NAD⁺-dependent protein deacetylase); this active ingredient may, for example, be resveratrol in suitable formulations, or those compounds as specified in WO2007019416 (e.g. SRT-1720), WO2008073451.

In one embodiment of the invention, the further active ingredient is DM-71 (N-acetyl-L-cysteine with bethanechol).

In one embodiment, the compound of the formula I is administered in combination with antihypercholesterolemic compounds, as described, for example, in WO2007107587, WO2007111994, WO2008106600, WO2008113796.

In a further embodiment, the compound of the formula I is administered in combination with inhibitors of SREBP (sterol regulatory element-binding protein), as described, for example, in WO2008097835.

In another embodiment, the compound of the formula I is administered in combination with a cyclic peptide agonist of the VPAC2 receptor, as described, for example, in WO2007101146, WO2007133828.

In a further embodiment, the compound of the formula I is administered in combination with an agonist of the endothelin receptor, as described, for example, in WO2007112069.

In a further embodiment, the compound of the formula I is administered in combination with AKP-020 (bis(ethylmaltolato)oxovanadium(IV)).

In another embodiment, the compound of the formula I is administered in combination with tissue-selective androgen receptor modulators (SARM), as described, for example, in WO2007099200, WO2007137874.

In a further embodiment, the compound of the formula I is administered in combination with an AGE (advanced glycation endproduct) inhibitor, as described, for example, in JP2008024673.

In one embodiment of the invention, the further active ingredient is leptin; see, for example, “Perspectives in the therapeutic use of leptin”, Salvador, Javier; Gomez-Ambrosi, Javier; Fruhbeck, Gema, Expert Opinion on Pharmacotherapy (2001), 2(10), 1615-1622.

In another embodiment of the invention, the further active ingredient is metreleptin (recombinant methionyl-leptin) combined with pramlintide.

In a further embodiment of the invention, the further active ingredient is the tetrapeptide ISF-402.

In one embodiment, the further active ingredient is dexamphetamine or amphetamine.

In one embodiment, the further active ingredient is fenfluramine or dexfenfluramine.

In another embodiment, the further active ingredient is sibutramine or those derivatives as described in WO2008034142.

In one embodiment, the further active ingredient is mazindol or phentermin.

In a further embodiment, the further active ingredient is geniposidic acid (WO2007100104) or derivatives thereof (JP2008106008).

In one embodiment, the further active ingredient is a nasal calcium channel blocker, for example diltiazem, or those as described in U.S. Pat. No. 7,138,107.

In one embodiment, the further active ingredient is an inhibitor of sodium-calcium ion exchange, for example those as described in WO2008028958, WO2008085711.

In a further embodiment, the further active ingredient is a blocker of calcium channels, for example of CaV3.2 or CaV2.2, as described in WO2008033431, WO2008033447, WO2008033356, WO2008033460, WO2008033464, WO2008033465, WO2008033468, WO2008073461.

In one embodiment, the further active ingredient is a modulator of a calcium channel, for example those as described in WO2008073934, WO2008073936.

In one embodiment, the further active ingredient is a blocker of the “T-type calcium channel”, as described, for example, in WO2008033431, WO2008110008.

In one embodiment, the further active ingredient is an inhibitor of KCNQ potassium channel 2 or 3, for example those as described in US2008027049, US2008027090.

In one embodiment, the further active ingredient is an inhibitor of the potassium Kv1.3 ion channel, for example those as described in WO2008040057, WO2008040058, WO2008046065.

In another embodiment, the further active ingredient is a modulator of the MCP-1 receptor (monocyte chemoattractant protein-1 (MCP-1)), for example those as described in WO2008014360, WO2008014381.

In one embodiment, the further active ingredient is a modulator of somatostatin receptor 5 (SSTR5), for example those as described in WO2008019967, US2008064697, US2008249101, WO2008000692.

In one embodiment, the further active ingredient is a modulator of somatostatin receptor 2 (SSTR2), for example those as described in WO2008051272.

In one embodiment, the further active ingredient is an erythropoietin-mimetic peptide which acts as an erythropoietin (EPO) receptor agonist. Such molecules are described, for example, in WO2008042800.

In a further embodiment, the further active ingredient is an anorectic/a hypoglycemic compound, for example those as described in WO2008035305, WO2008035306, WO2008035686.

In one embodiment, the further active ingredient is an inductor of lipoic acid synthetase, for example those as described in WO2008036966, WO2008036967.

In one embodiment, the further active ingredient is a stimulator of endothelial nitric oxide synthase (eNOS), for example those as described in WO2008058641, WO2008074413.

In one embodiment, the further active ingredient is a modulator of carbohydrate and/or lipid metabolism, for example those as described in WO2008059023, WO2008059024, WO2008059025, WO2008059026.

In a further embodiment, the further active ingredient is an angiotensin II receptor antagonist, for example those as described in WO2008062905, WO2008067378.

In one embodiment, the further active ingredient is an agonist of the sphingosine-1-phosphate receptor (SIP), for example those as described in WO2008064315, WO2008074820, WO2008074821.

In one embodiment, the further active ingredient is an agent which retards gastric emptying, for example 4-hydroxyisoleucine (WO2008044770).

In one embodiment, the further active ingredient is a muscle-relaxing substance, as described, for example, in WO2008090200.

In a further embodiment, the further active ingredient is an inhibitor of monoamine oxidase B (MAO-B), for example those as described in WO2008092091.

In another embodiment, the further active ingredient is an inhibitor of the binding of cholesterol and/or triglycerides to the SCP-2 protein (sterol carrier protein-2), for example those as described in US2008194658.

In another embodiment, the further active ingredient is lisofylline, which prevents autoimmune damage to insulin-producing cells.

In one embodiment, the compound of the formula I is administered in combination with bulking agents, preferably insoluble bulking agents (see, for example, Carob/Caromax®(Zunft H J; et al., Carob pulp preparation for treatment of hypercholesterolemia, ADVANCES IN THERAPY (2001 September-October), 18(5), 230-6). Caromax is a carob-containing product from Nutrinova, Nutrition Specialties & Food Ingredients GmbH, Industriepark Hochst, 65926 Frankfurt/Main)). Combination with Caromax® is possible in one preparation or by separate administration of compounds of the formula I and Caromax®. Caromax® can in this connection also be administered in the form of food products such as, for example, in bakery products or muesli bars.

It will be appreciated that every suitable combination of the compounds of the invention with one or more of the aforementioned compounds and optionally one or more other pharmacologically active substances is considered to be covered within the scope of protection conferred by the present invention.

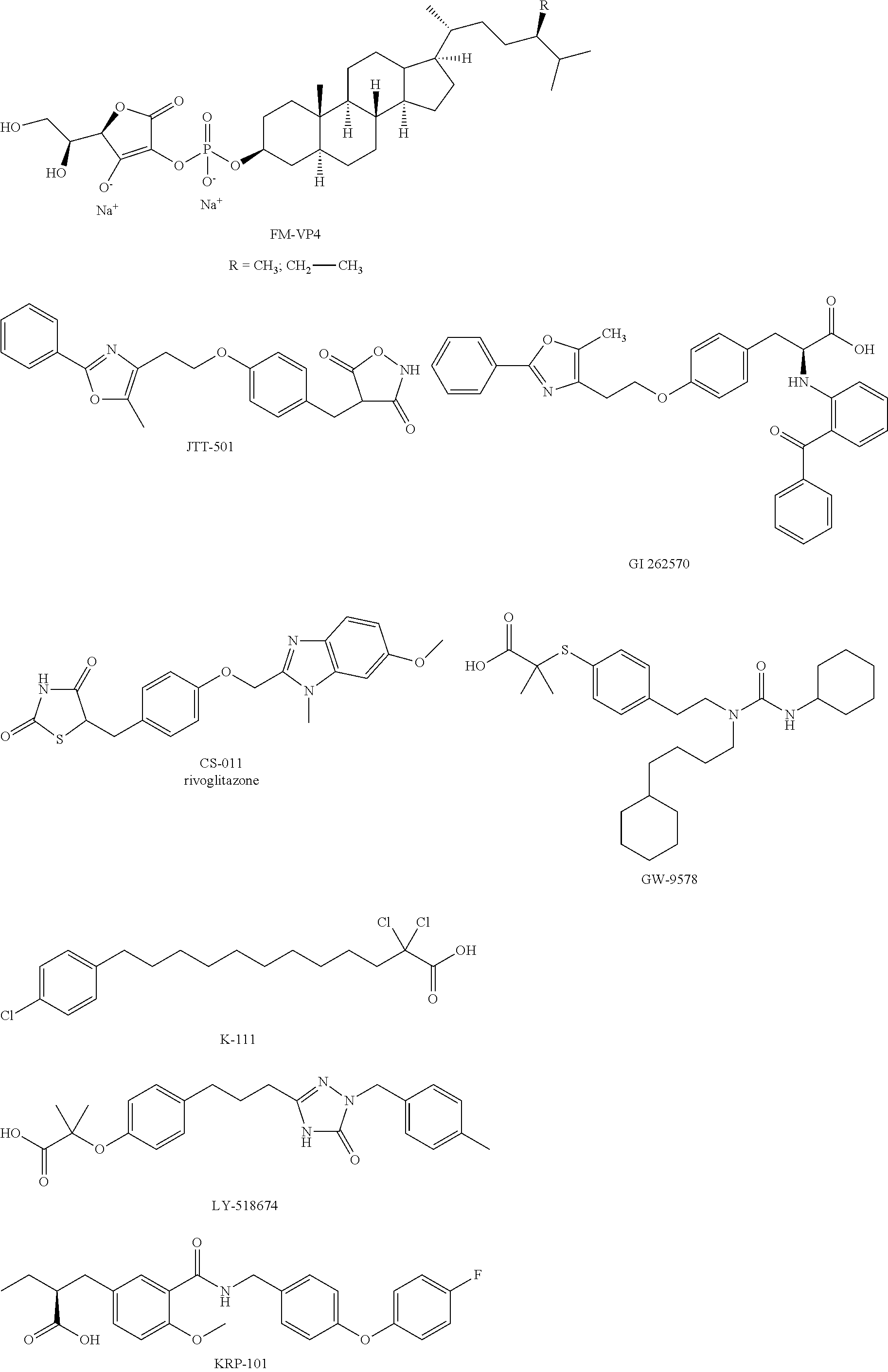

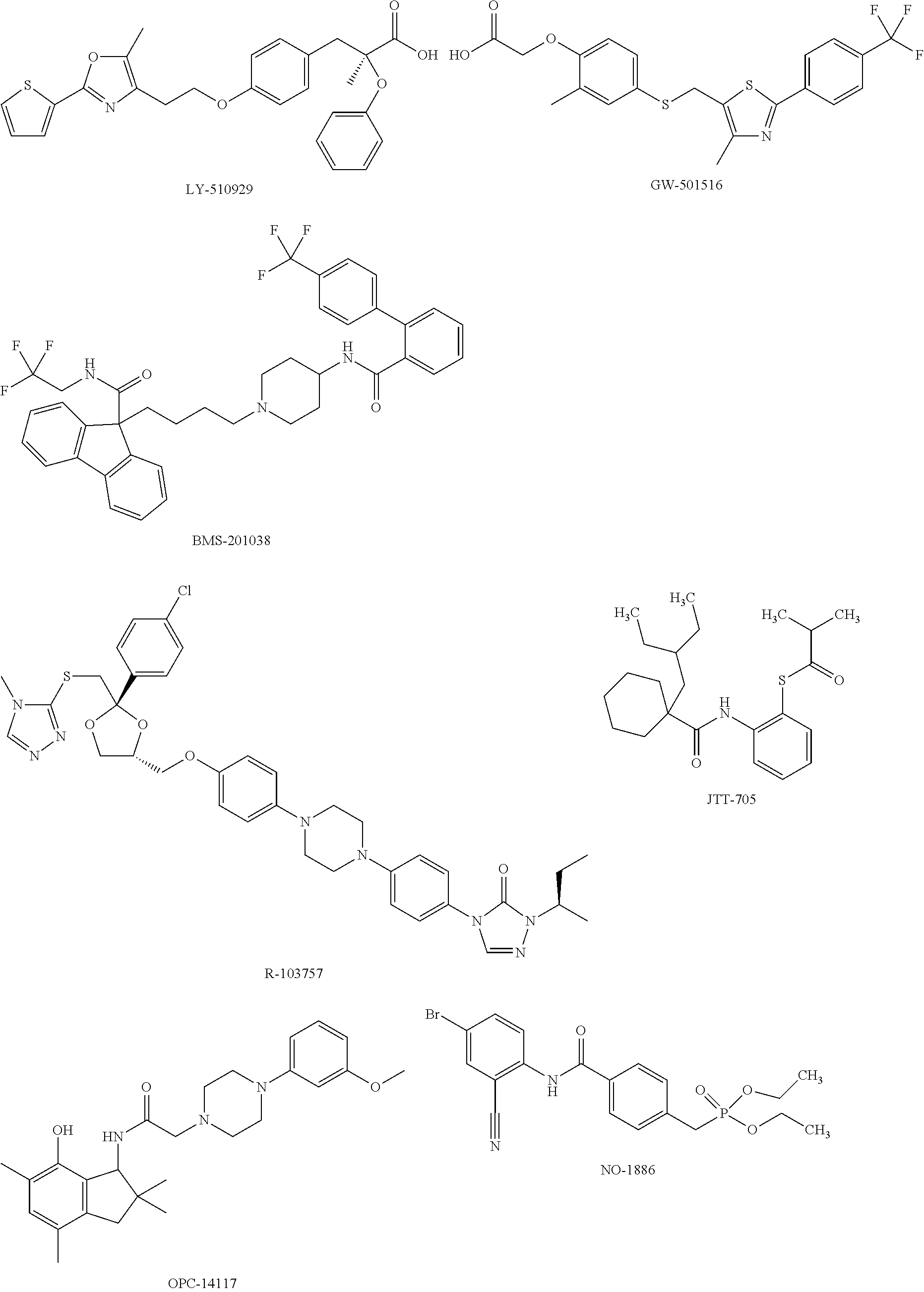

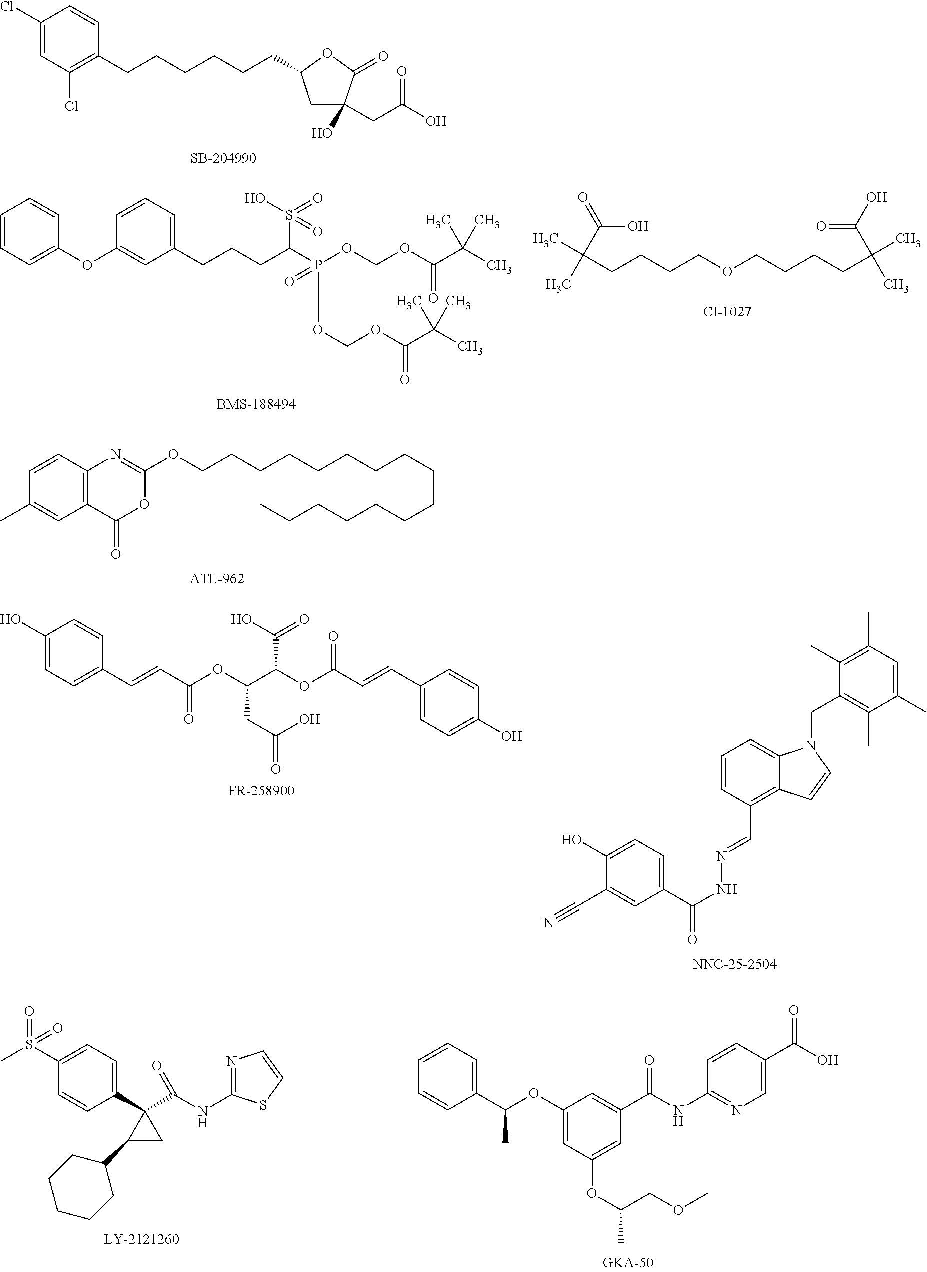

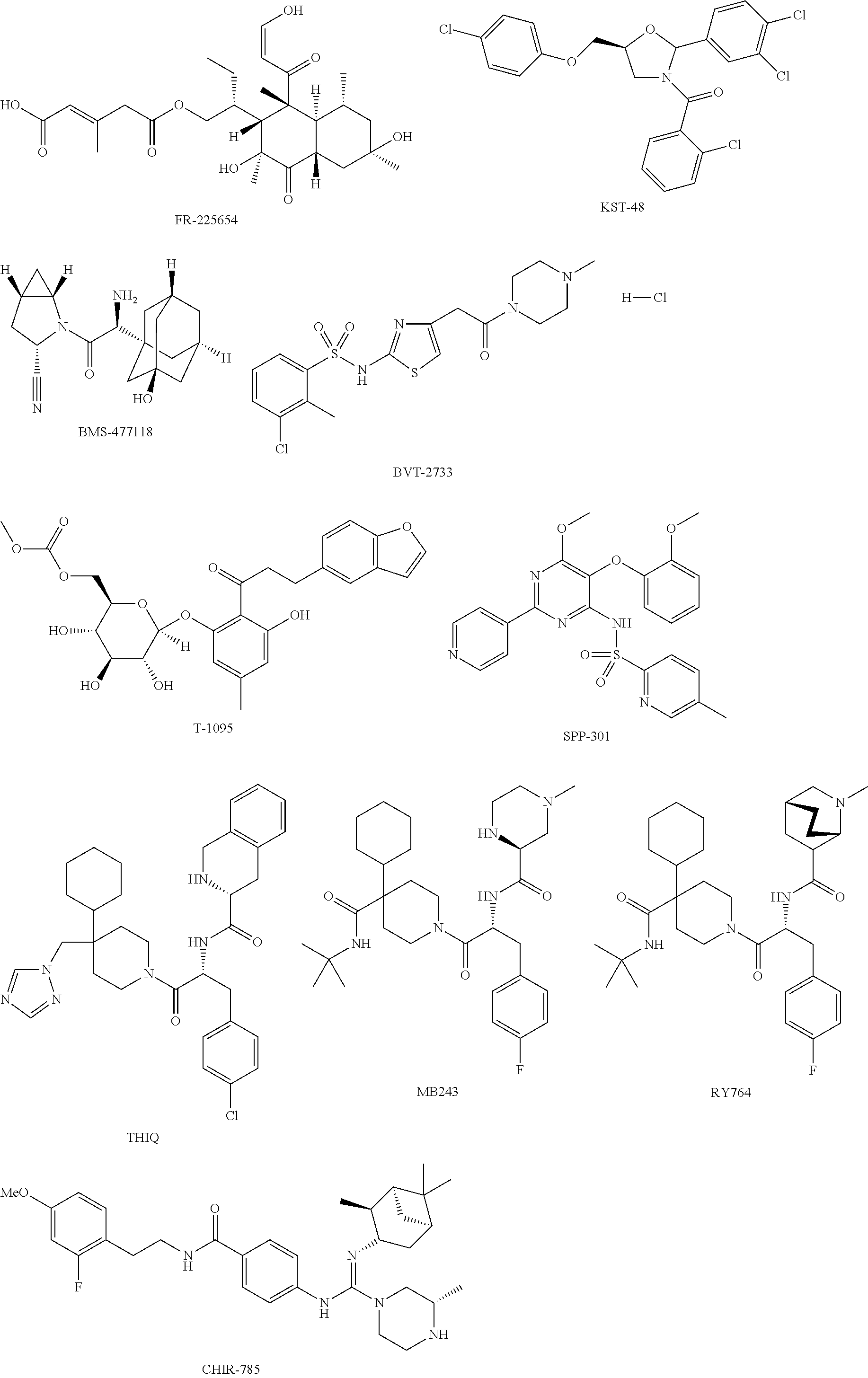

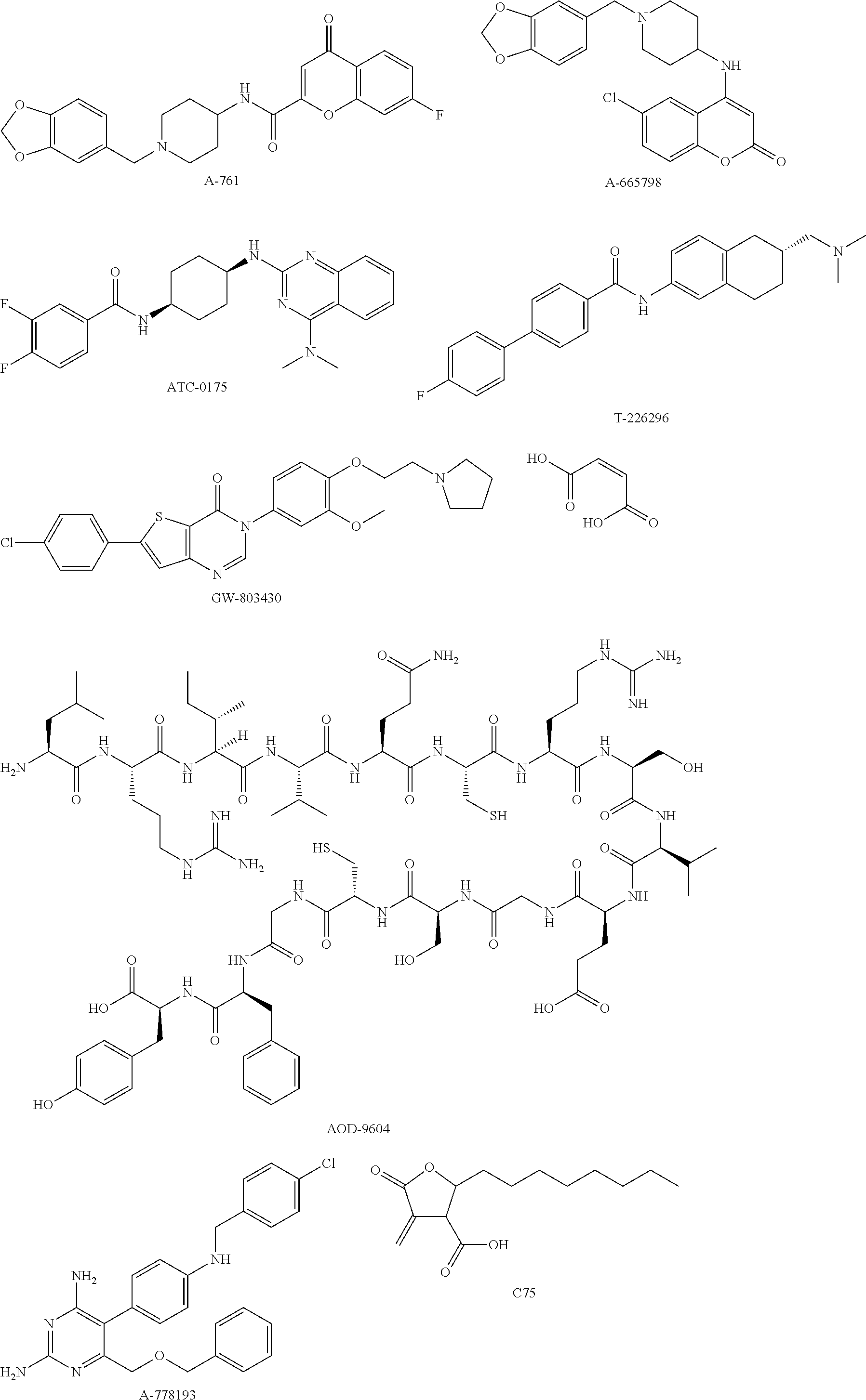

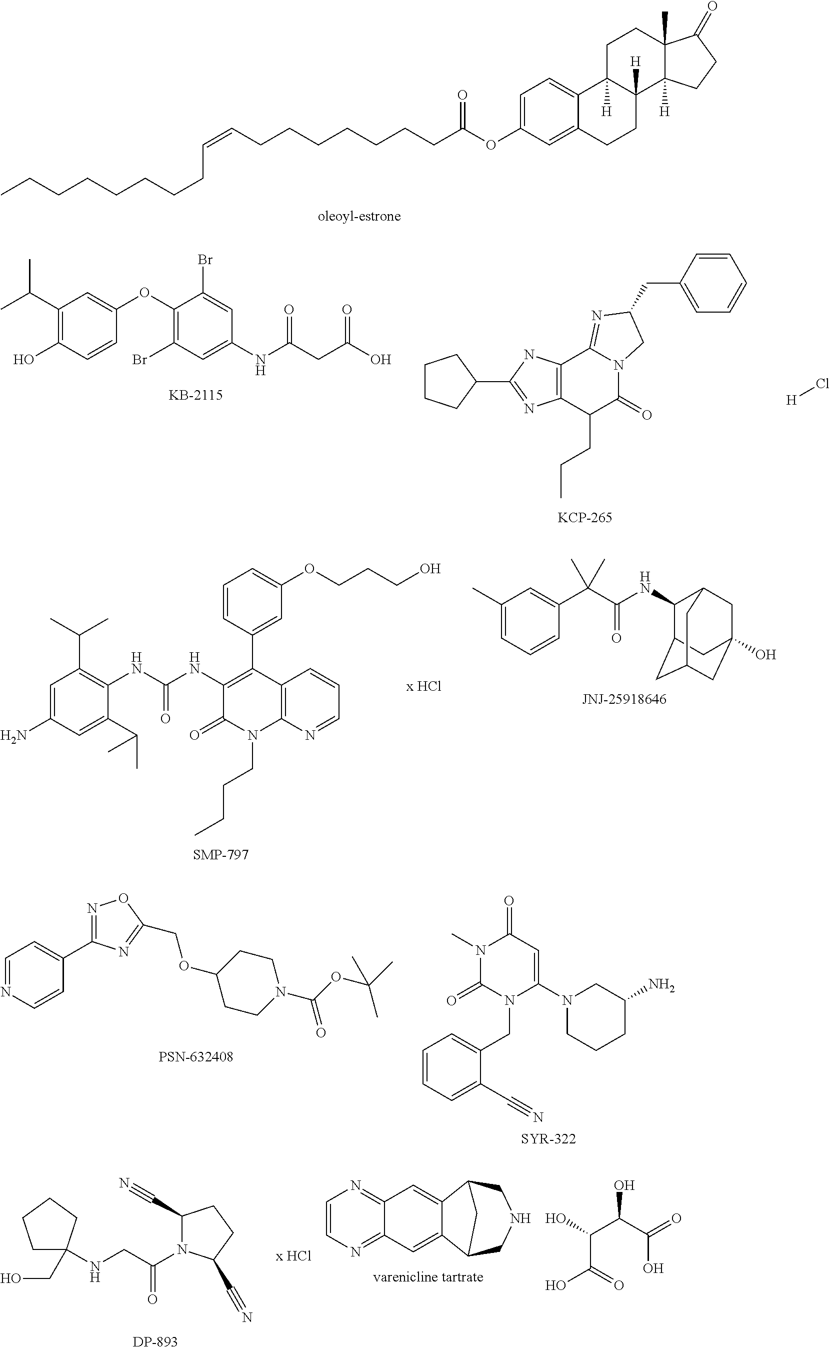

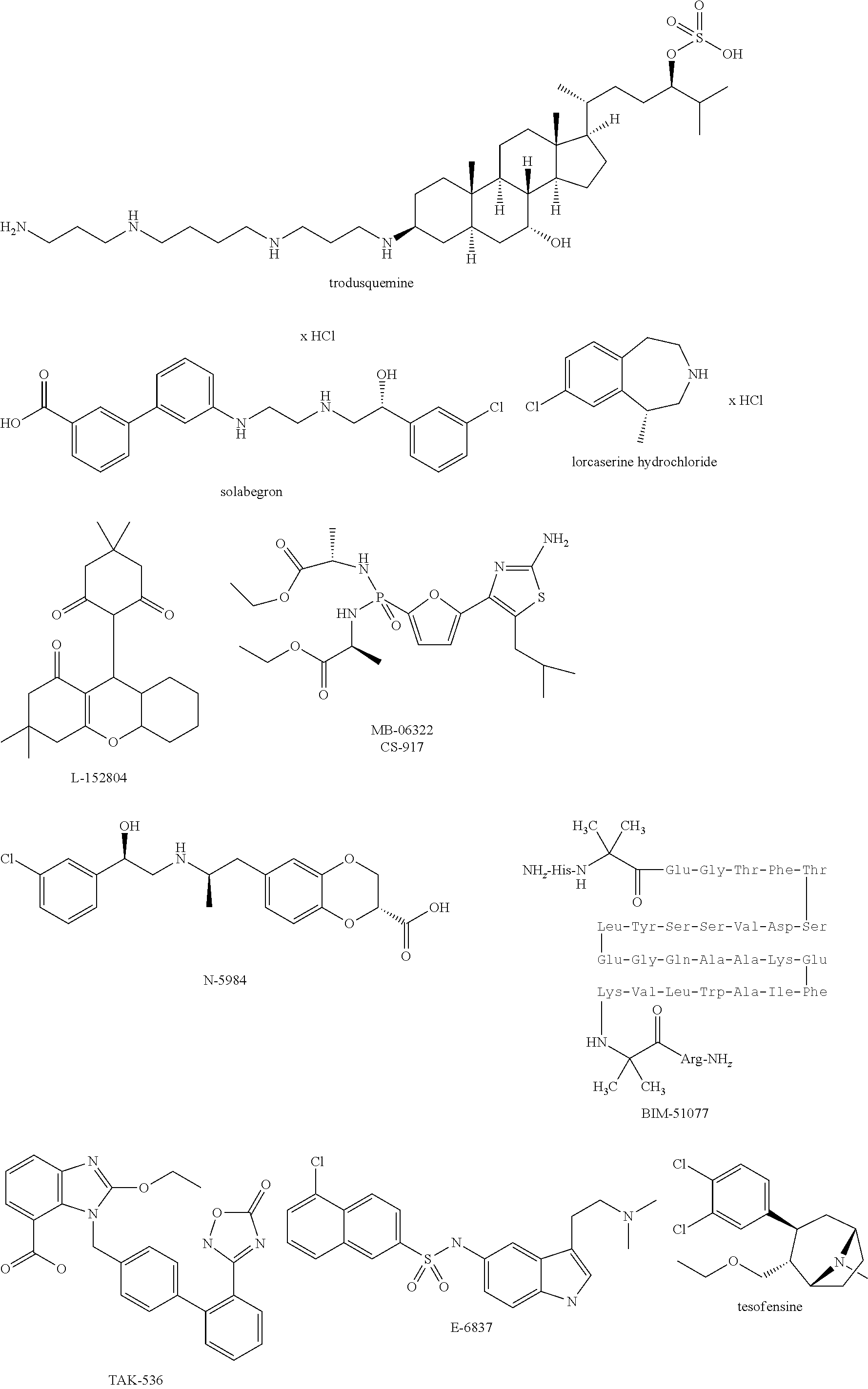

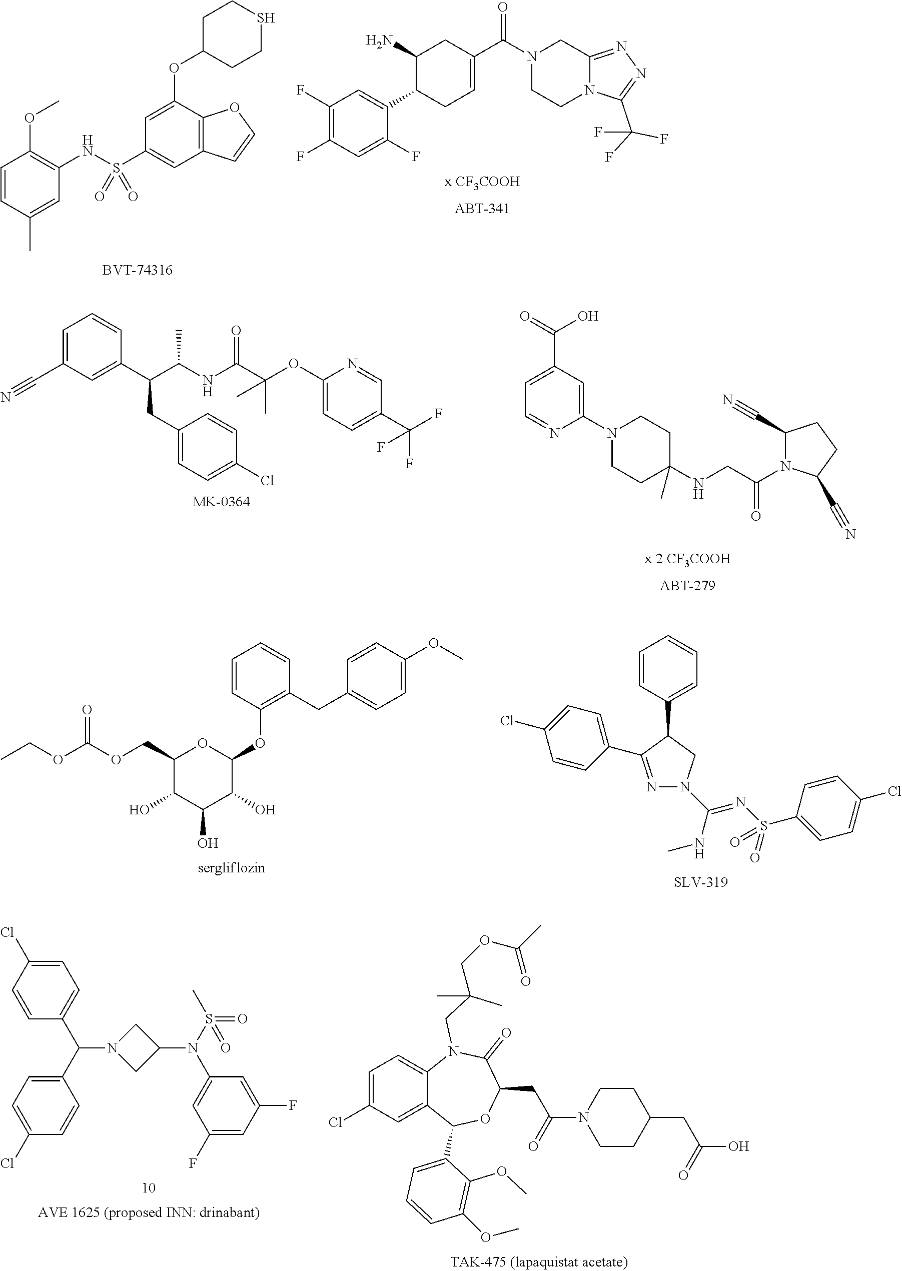

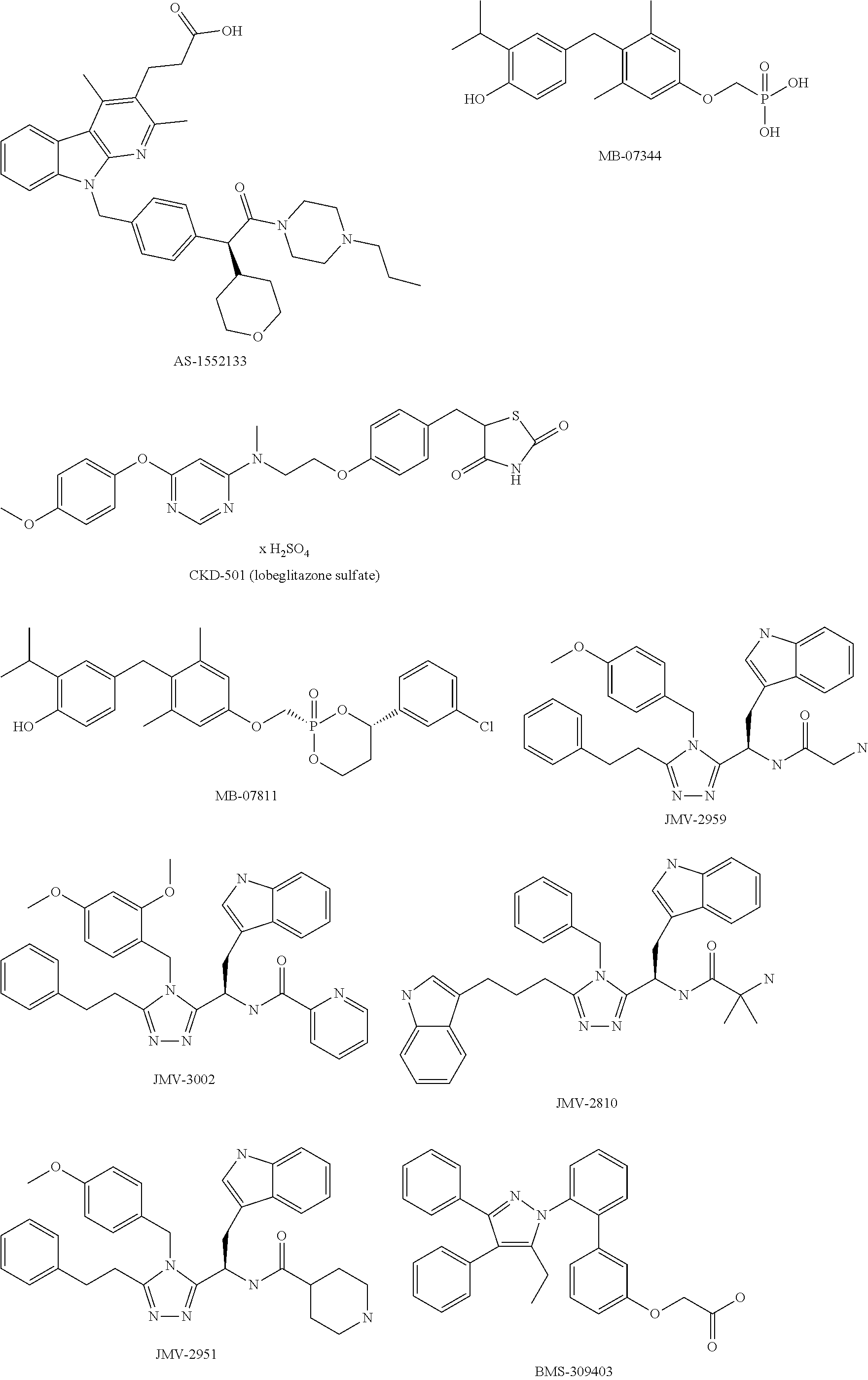

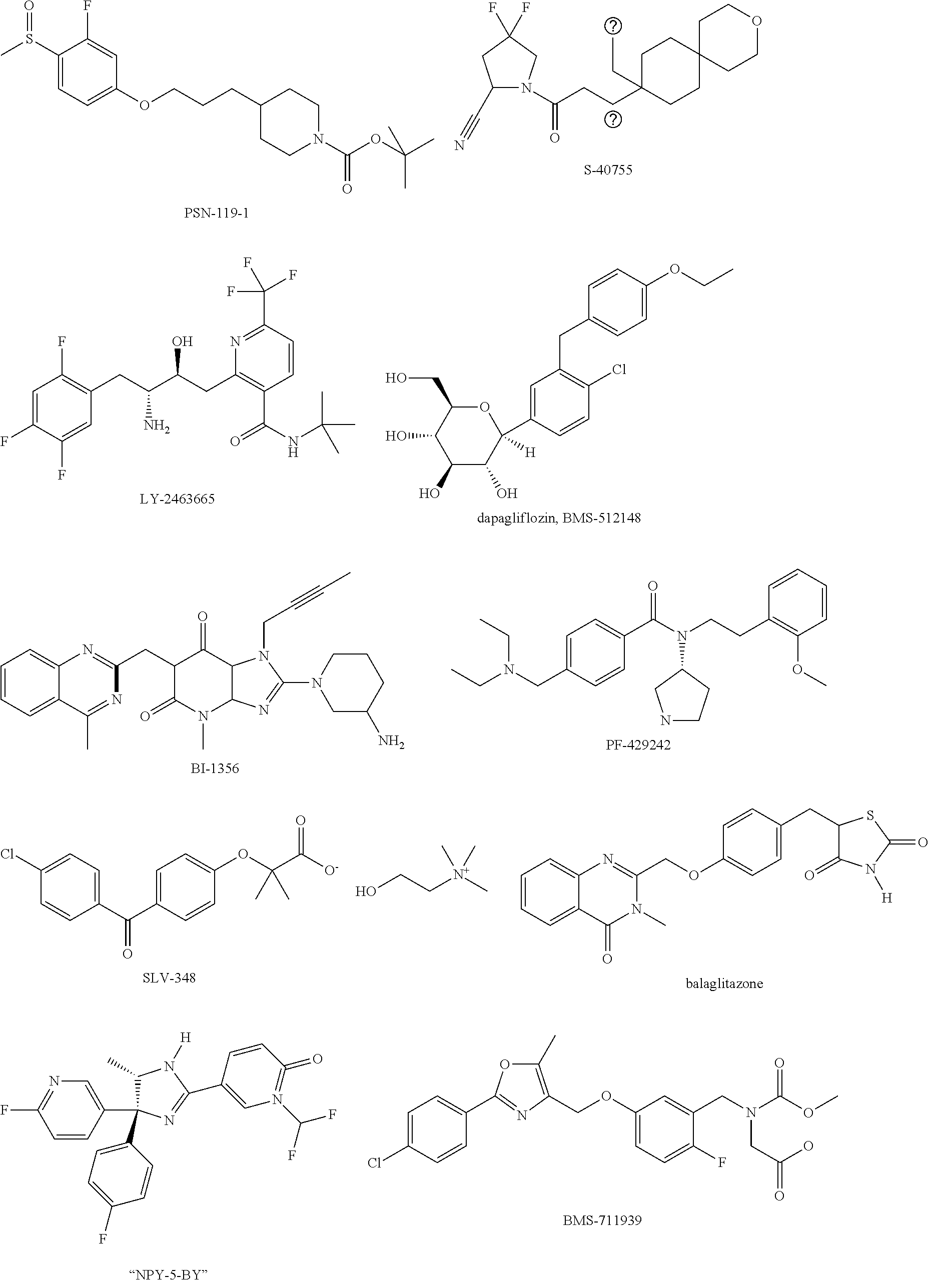

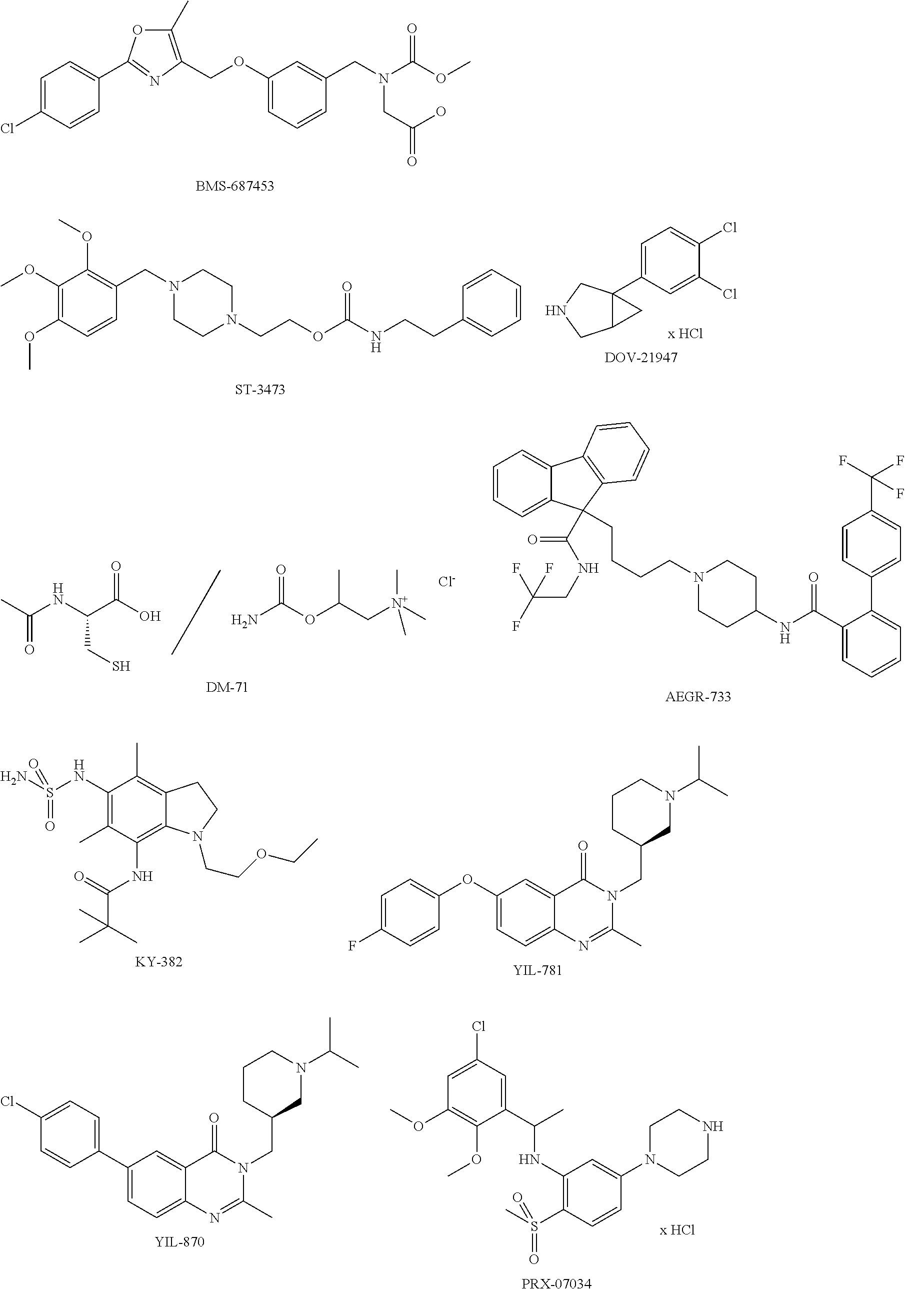

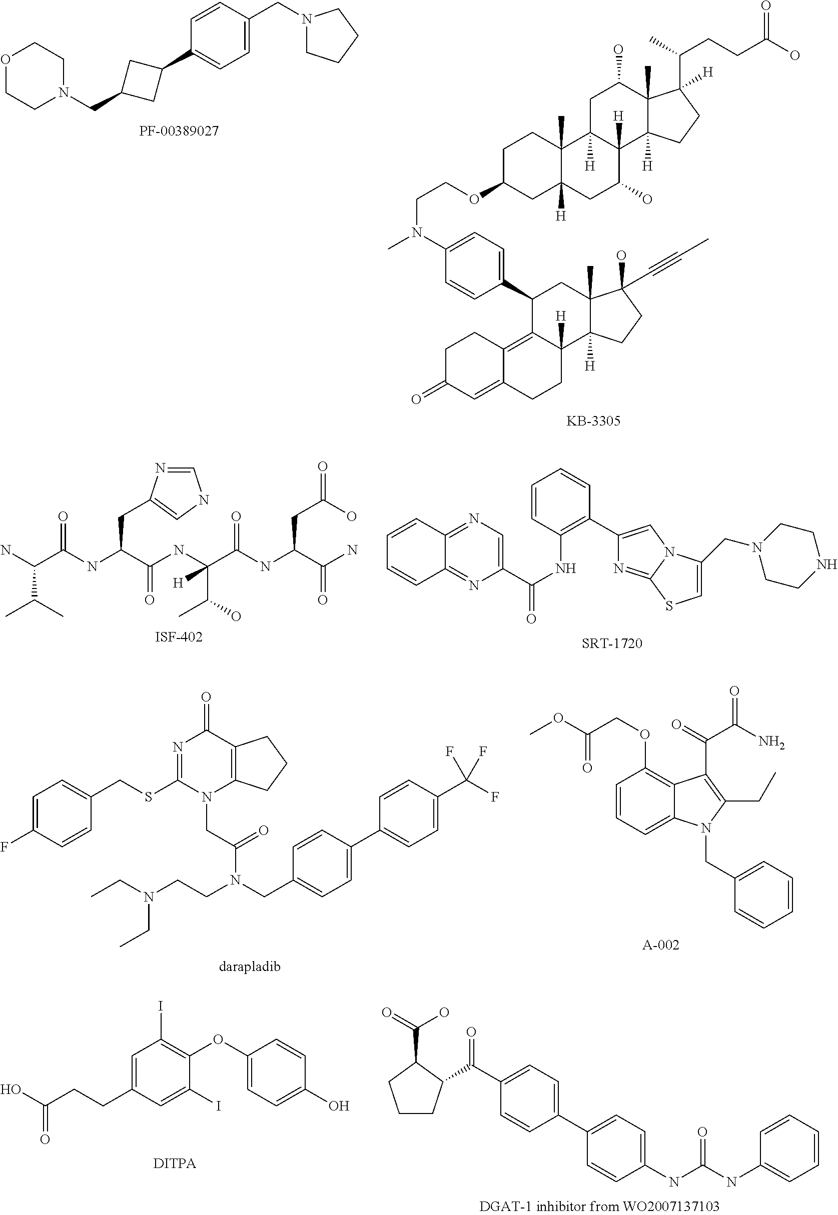

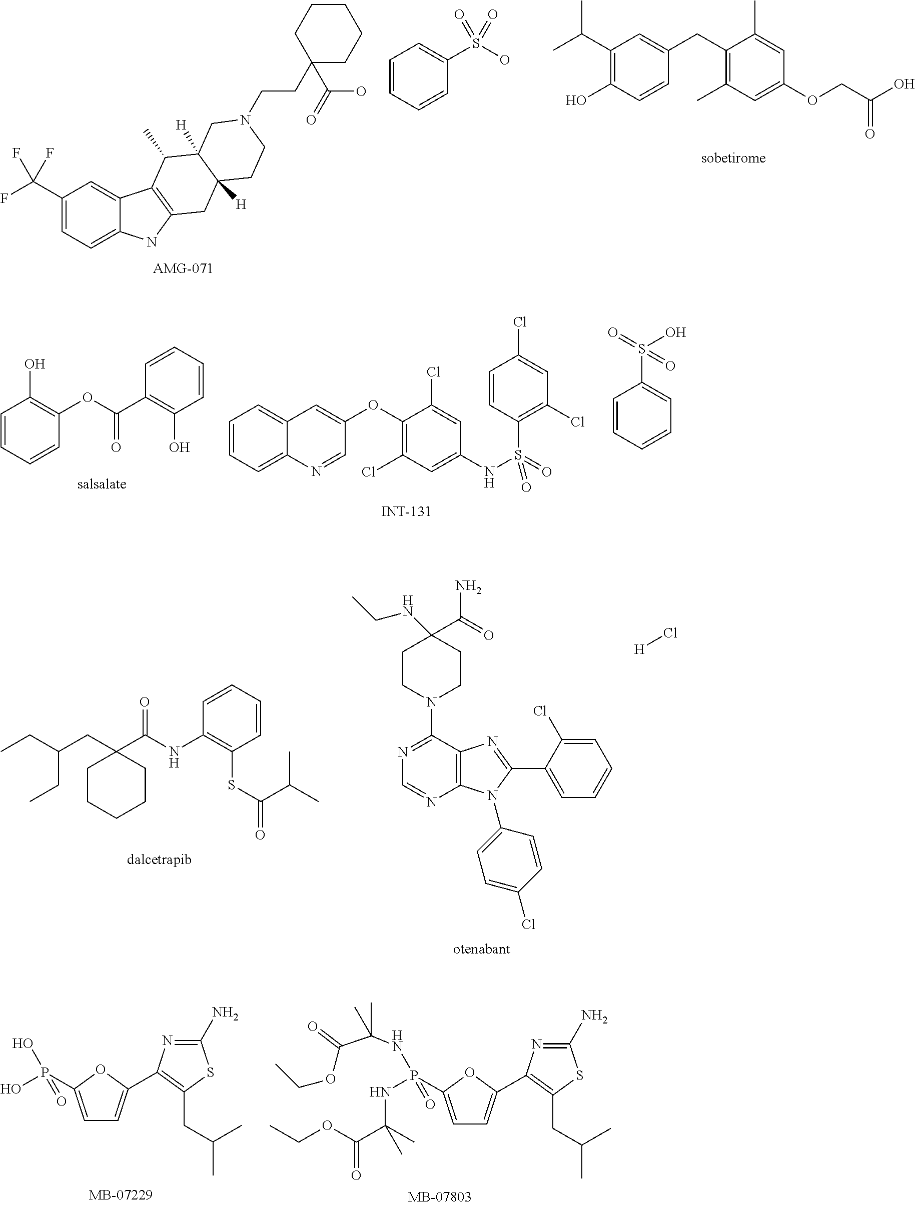

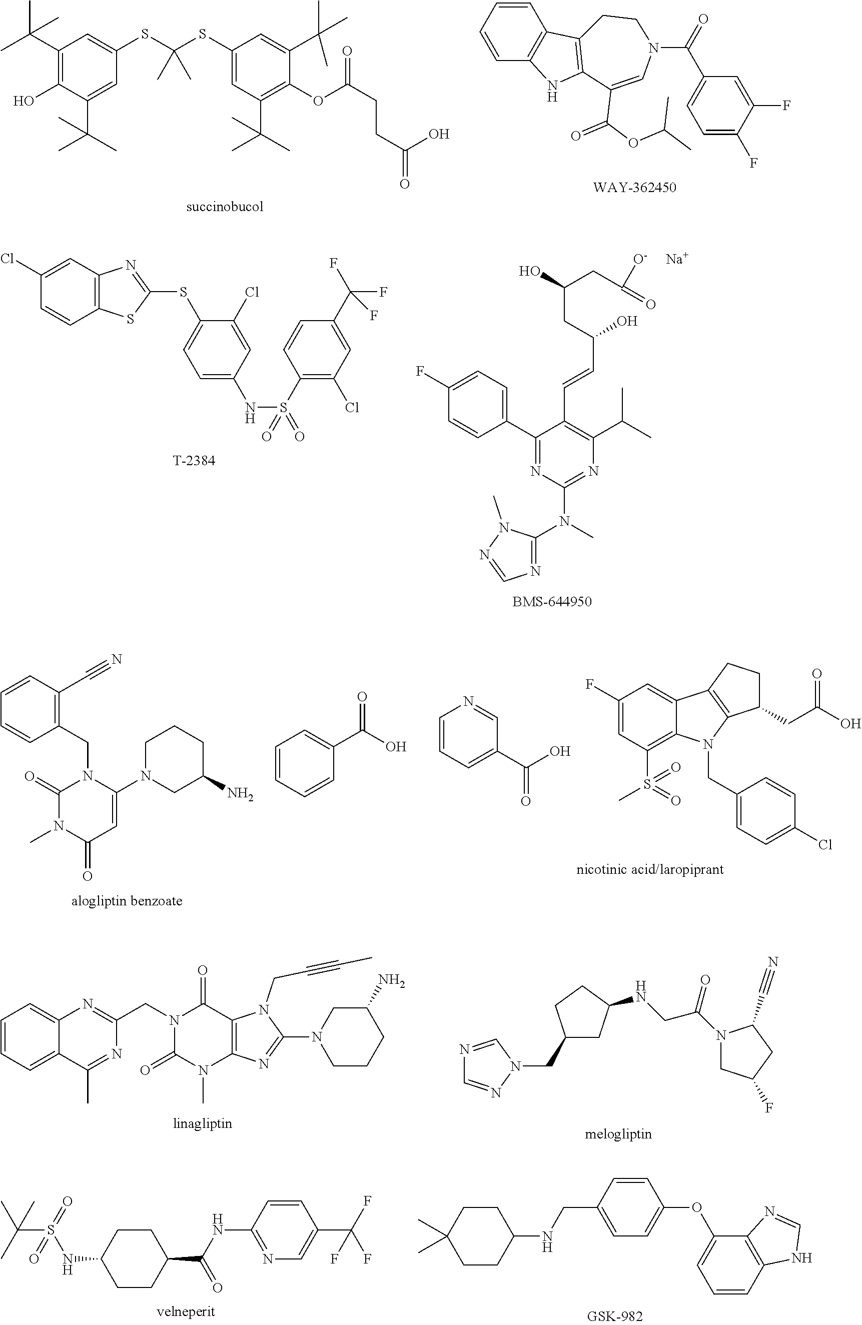

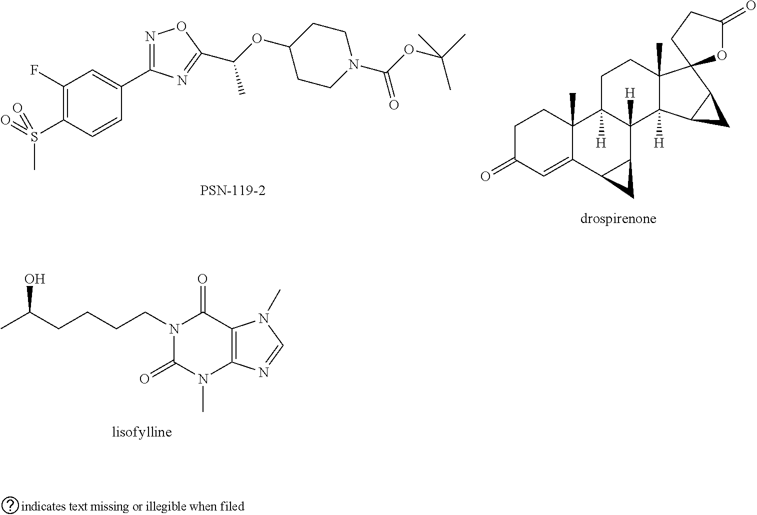

Also suitable are the following active ingredients for combination preparations:

all antiepileptics specified in the Rote Liste 2007, chapter 15;
all antihypertensives specified in the Rote Liste 2007, chapter 17;
all hypotonics specified in the Rote Liste 2007, chapter 19;
all anticoagulants specified in the Rote Liste 2007, chapter 20;
all arteriosclerosis drugs specified in the Rote Liste 2007, chapter 25;
all beta receptors, calcium channel blockers and inhibitors of the renin angiotensin system specified in the Rote Liste 2007, chapter 27;
all diuretics and perfusion-promoting drugs specified in the Rote Liste 2007, chapter 36 and 37;
all withdrawal drugs/drugs for the treatment of addictive disorders specified in the Rote Liste 2007, chapter 39;
all coronary drugs and gastrointestinal drugs specified in the Rote Liste 2007, chapter 55 and 60;
all migraine drugs, neuropathy preparations and Parkinson's drugs specified in the Rote Liste 2007, chapter 61, 66 and 70.

The invention further provides processes for preparing the compounds of the general formula I, wherein the compounds of the formula I are obtained in a procedure analogous to the reaction schemes which follow.

### Method “A”:

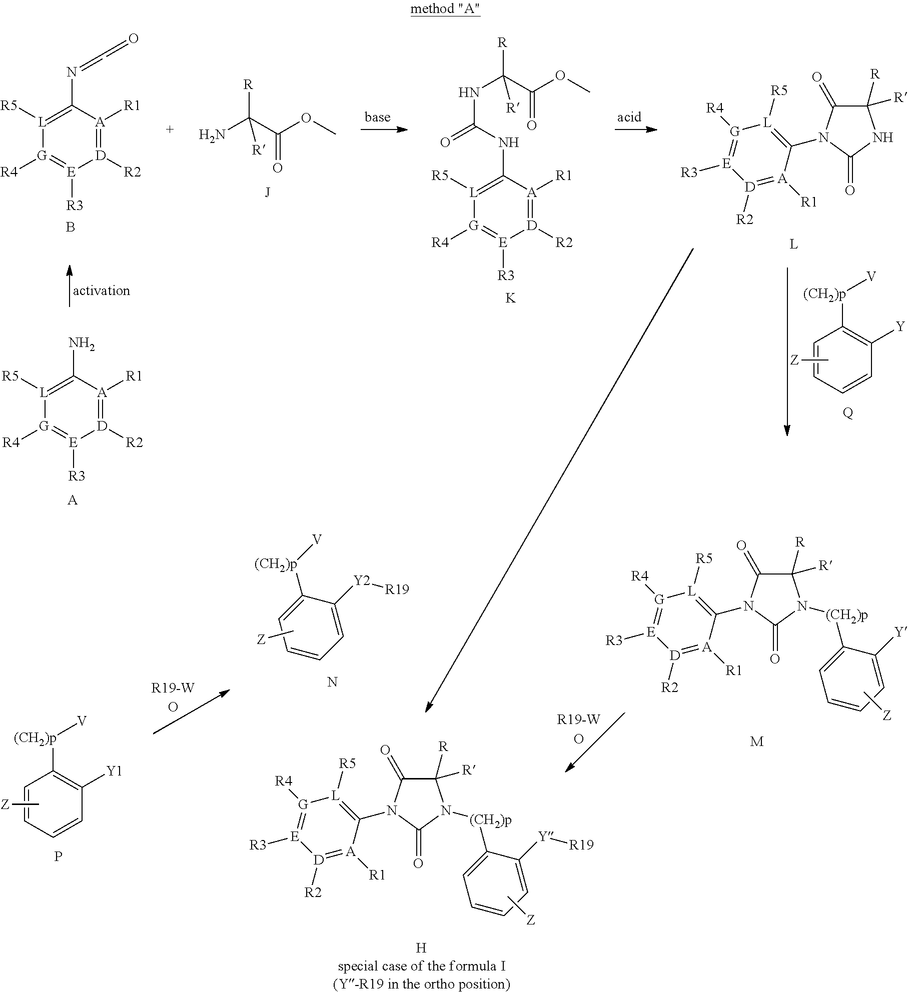

In a first method “A”, the procedure is to convert a suitably substituted aniline of the formula A in which the R1 to R5 radicals are in some circumstances present in protected form to an isocyanate of the formula B. This reaction can be carried out, for example, with phosgene in toluene or with diphosgene or triphosgene. The isocyanate B is subsequently reacted with the methyl ester or another ester (e.g. tert-butyl) of the amino acid J, in which R and R′ are each as defined in formula I, or a salt of an ester of the amino acid J with addition of base (e.g. triethylamine) to give a urea of the formula K. This urea can be ring-closed under basic or acidic conditions, preferably acidic conditions, to give the imidazolidine-2,4-dione of the formula L. The further conversion to a compound of the formula H, which constitutes the ortho-substituted special case of a compound of the formula I, can, for example, be effected by alkylating a suitably substituted compound Q where Z may be one or more substituents as described above in formula I, and Y is either a nitro function (—NO₂) or a halogen atom, preferably a bromine atom, or else a suitably protected amino function (e.g. isoindo1-1,3-dion-2-yl or N═CH—N(CH₃)₂), and V is either also a halogen atom, preferably a chlorine or bromine atom, or else, for example, an O—SO₂—C₆H₄-4-CH₃ radical or an O—SO₂—CH₃ radical or an O—SO₂—CF₃ radical, to obtain the compound M. When Y′ in M is nitro (NO₂), it can be converted by reduction to a compound M where Y′ is amino (NH₂). When Y′ in M is a protected amino function, it can be converted to a free amino function by protecting group-specific elimination. When Y′ in M is a halogen atom, preferably a bromine atom, it can be converted under Buchwald-Hartwig conditions (e.g.: S. L. Buchwald et al.: Acc. Chem. Res. 1998, 31, 805; J. F. Hartwig et al.: J. Org. Chem. 1999, 64, 5575-5580; J. P. Wolfe et al.: J. Org. Chem. 2000, 65, 144-1157; M. D. Charles et al.: Org. Lett. 2005, 7, 3965-68), for example by reaction with benzophenone imine and subsequent hydrolysis, to a compound of the formula M where Y′ is amino (NH₂). The further conversion to compounds of the formula H can be effected by reaction with isocyanates of the formula O where W is —N═C═O.

Alternatively, a compound of the formula Min which Y′ is NH₂ can be converted using, for example, phosgene or phosgene equivalents, to an isocyanate in which Y′ is —N═C═O, which can subsequently be reacted with a compound of the formula O in which W is NH₂. Or the further conversion of the compound L to the compound H can be effected by reacting L under alkylating conditions with a compound of the formula N where V may be defined as just outlined, and where Y2 may be defined as NR23-CO—NR23 (in the case of the urea-bridged compounds). The compound N in turn can be obtained by reaction of P in which V may be defined as described above, and where Y1 is NH₂, with a possibly substituted R19—W compound O in which W is defined as —N═C═O. R19 is defined as substituted or unsubstituted aryl, heteroaryl or bicyclic heteroaryl.

Any protecting groups present in the compound H can be removed at the end.

The formula H shown here constitutes a special case of the formula I in which the Y″—R19 radical in formula I is in the ortho position; this radical may correspondingly also be in the meta or para position.

The process described here constitutes the special case in which Y2 or Y″ describes a urea bridge (—NR23-CO—NR24—). The process can also be applied correspondingly to other compounds bridged with “T”. The definition of “T” is described above.

One variant of method “A” is that of method “N”:

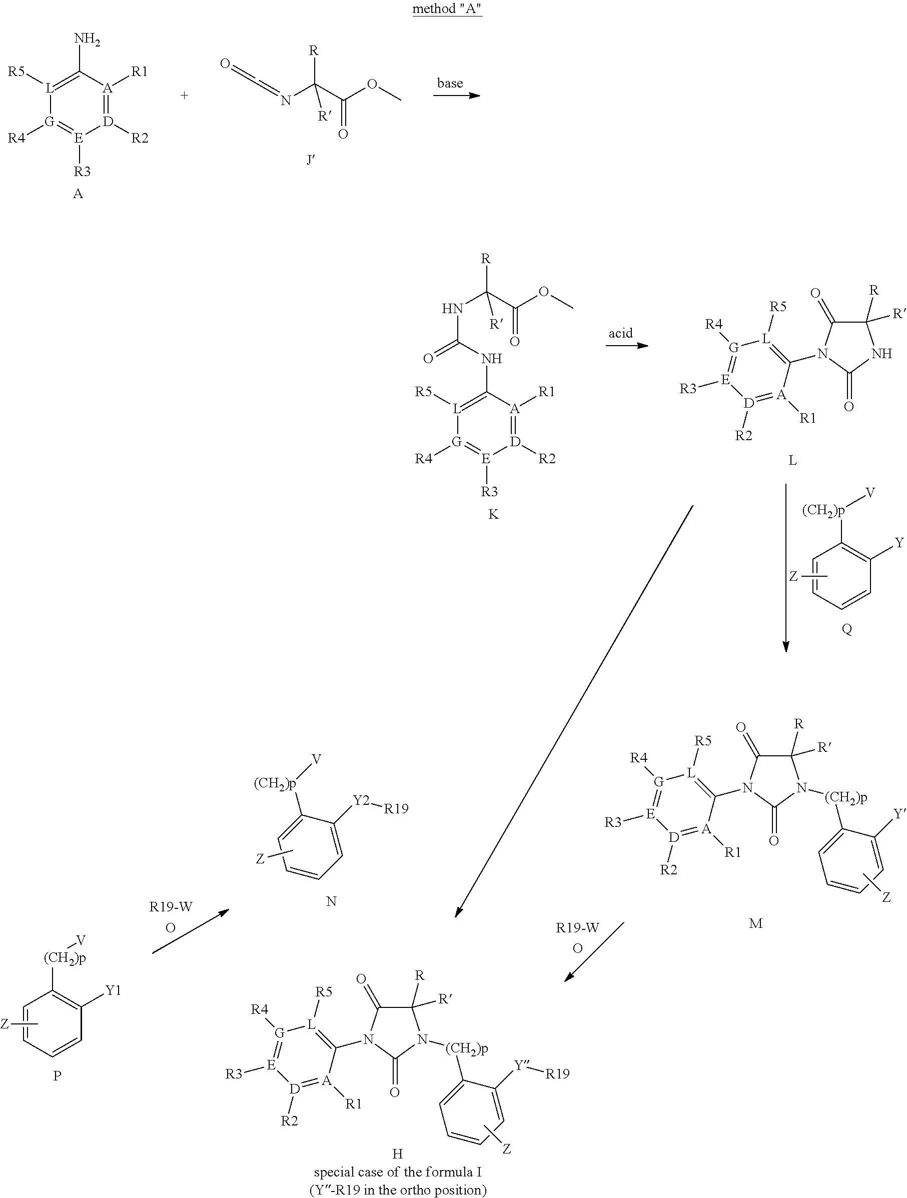

In method “A′”, the amine A is reacted with the isocyanate of the amino acid ester J′ to form the compound K. The further steps can be effected as in method “A”.

In another method “B”,

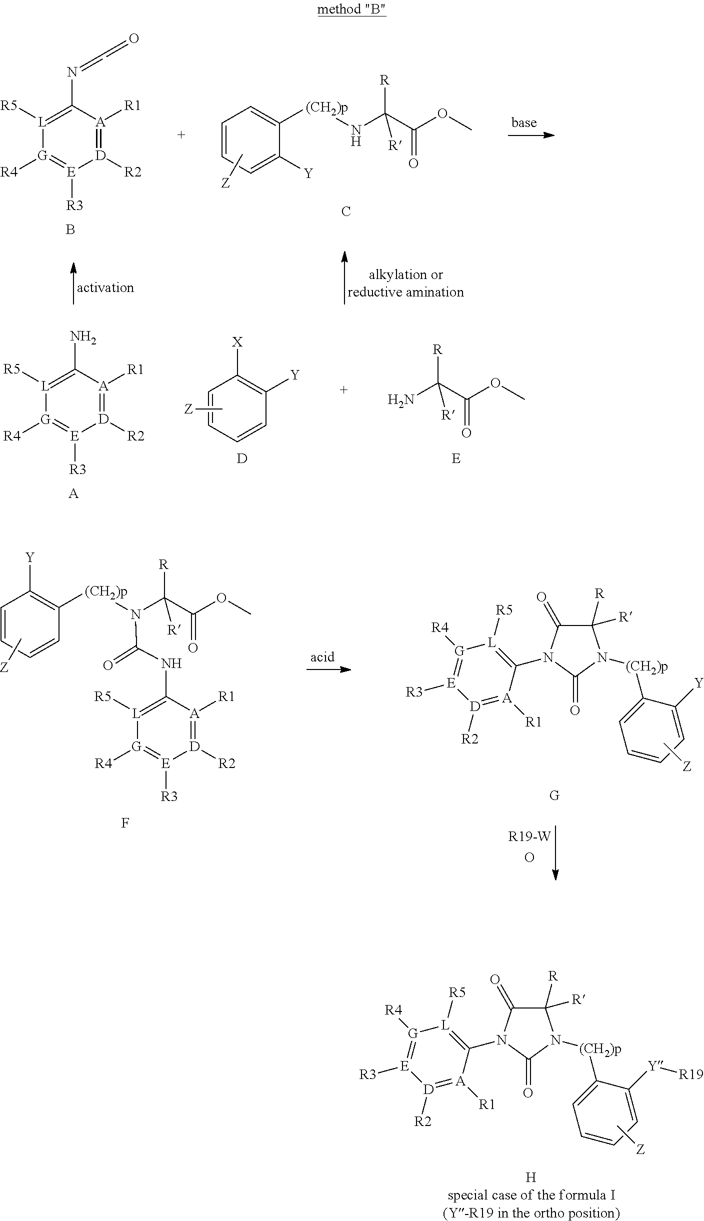

the isocyanate B is reacted with a suitably substituted amino acid ester derivative C in which the particular substituents may be provided with protecting groups, and where the methyl ester shown in the scheme is a nonlimiting example of an ester, and where Y is bromine or a protected amino function (e.g. N—CO—CH₃ or N═CH—N(CH₃)₂), with addition of a base (e.g. triethylamine), to give a urea of the formula F. The amino acid ester derivative C can be prepared from the compound D in which Z may be one or more substituents as described above in formula I, and where Y is bromine or a protected amino function and X is a (CH₂)ₚ-U moiety in which U may be defined as Cl, Br, I, O—SO₂—C₆H₄-4-CH₃, O—SO₂—CH₃ or O—SO₂—CF₃, with an amino acid ester of the formula E in which R and R′ are each as defined in formula I, under alkylating conditions. Alternatively, the compound of the formula C can be obtained by reductive amination of the aldehyde D (Z and Y as described above and X═(CH₂)₍ₚ₋₁₎—CHO with the amino acid derivative E. The urea F can be ring-closed under basic or acidic conditions, preferably acidic conditions, to give the imidazolidine-2,4-dione of the formula G. Compounds of the formula G in which Y is bromine can be converted to compounds of the formula G in which Y is NH₂ by the method described in method “A”. The conversion of G to H can subsequently be carried out by the methods described in method “A”.

Any protecting groups present in the compound H can be removed at the end.

The formula H shown here constitutes a special case of the formula I in which the Y″—R19 radical in formula I is in the ortho position; this radical may correspondingly also be in the meta or para position.

The process described here constitutes the special case in which Y″ describes a urea bridge (—NR23-CO—NR24—). The process can also be applied correspondingly to other compounds bridged with “T”. The definition of “T” is described above.

In a further method (method “C”),

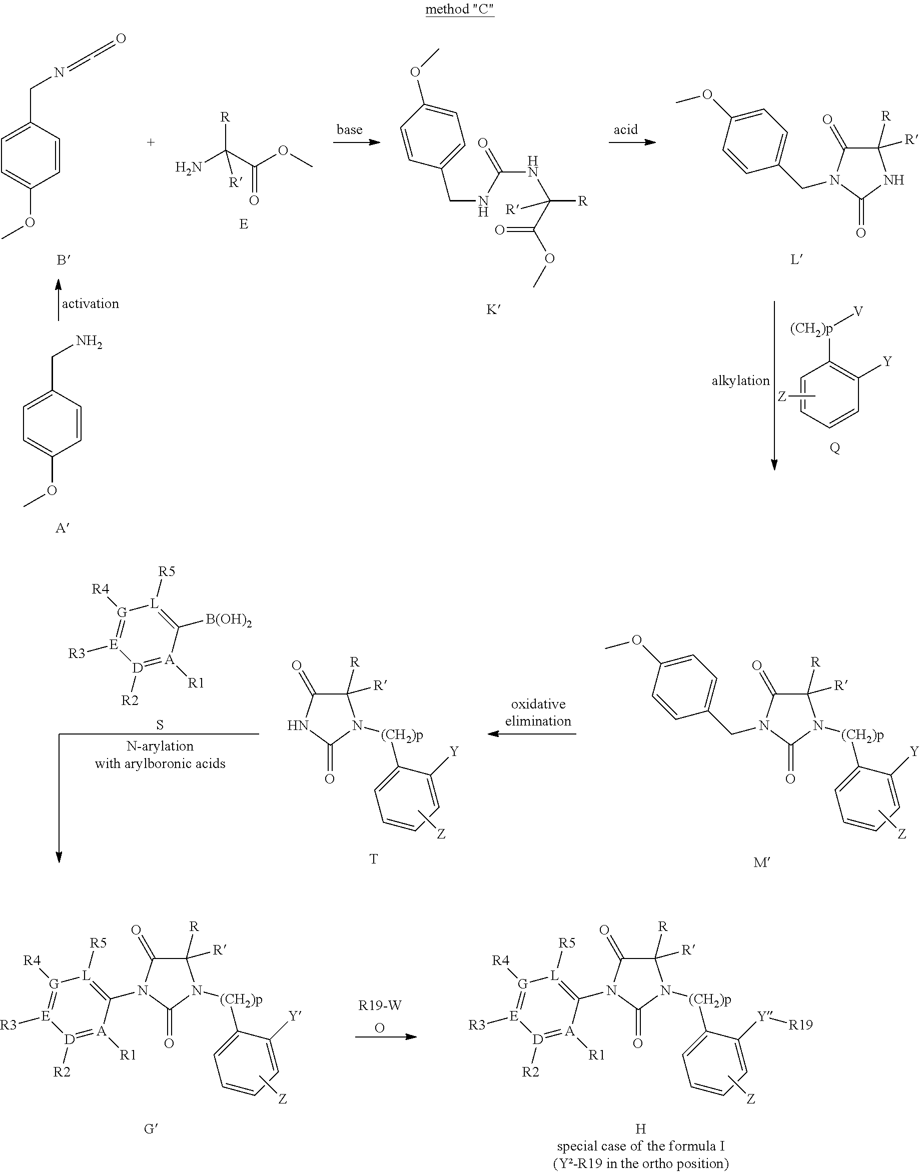

p-methoxybenzyl isocyanate B′ is reacted with an amino acid ester, for example E, in which R and R′ are each as defined in formula I, under basic conditions to give the urea K′. The urea K′ can be ring-closed under basic or acidic conditions, preferably acidic conditions, to give the imidazolidine-2,4-dione of the formula L′. The compounds M′ are obtained by reacting the compounds L′ with the compounds Q under alkylating conditions. Z, V and Y of the compounds Q are each defined as specified in method “A”. The p-methoxybenzyl group in the compounds M′ can be eliminated oxidatively to obtain the compounds T. The N-arylation of the imide nitrogen atom in compounds of the formula T using arylboronic acids of the formula S by processes as described, for example, in J.-B. Lan et al.: SYNLETT 2004, 1095-1097 or D. M. T. Chan et al.: Tetrahedron Lett. 1998, 39, 2933-2936, affords compounds of the formula G′. Compounds of the formula G′ can be converted to compounds of the formula H under the different conditions as specified in method “A” for the conversion of M to H. Any protecting groups present in the compound H can be removed at the end. The formula H shown here constitutes a special case of the formula I in which the Y″—R19 radical in formula I is in the ortho position; this radical may correspondingly also be in the meta or para position.

The process described here constitutes the special case in which Y″ describes a urea bridge (—NR23-CO—NR24—). The process can also be applied correspondingly to other compounds bridged with “T”. The definition of “T” is described above.

A further method “D” finds use especially in the synthesis of alkyl-, cycloalkyl-, cycloalkenyl-, arylalkylene-, heteroarylalkylene-, aryloxy-, heteroaryloxy-, alkyloxy-, alkylthio-, cycloalkylthio-, arylthio-, heteroarylthio-, alkylcarbonyl-, cycloalkylcarbonyl-, arylcarbonyl-, heteroarylcarbonyl-, aryl- and heteroaryl-substituted N3-aryl- or N3-heteroaryl-substituted imidazolidine-2,4-diones.

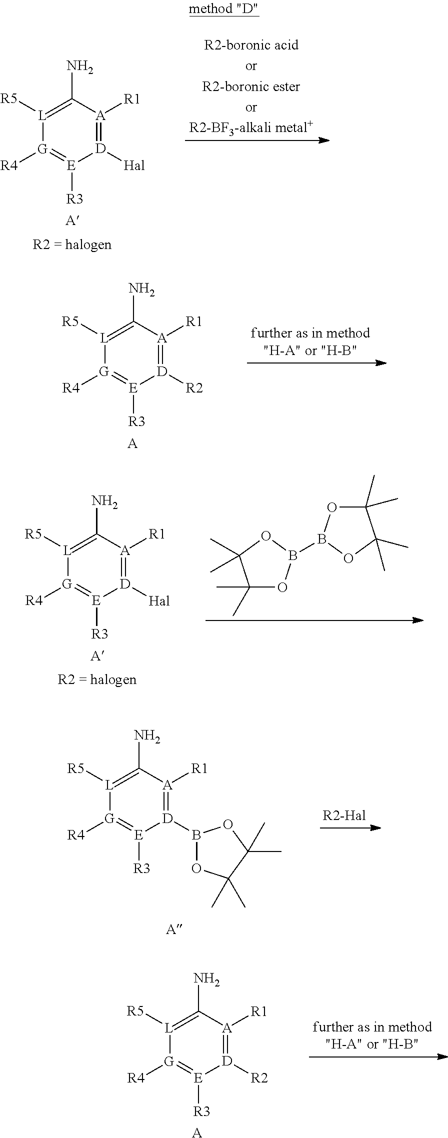

To prepare compounds in which, for example, R2=alkyl, cycloalkyl, cycloalkenyl, aryl or heteroaryl or another of the above-described radicals, the procedure may be to react a compound of the formula A′ in which the amino function may be provided with a protecting group and R2 is halogen, preferably bromine or chlorine, with an alkyl-, cycloalkyl-, cycloalkenyl-, aryl- or heteroarylboronic acid or an ester derivative thereof or an R2 trifluoroborate, under conditions as described, for example, in J. Zhou and G. C. Fu, J. Am. Chem. Soc. 126 (2004) 1340-1341; F. Gonzales-Bobes and G. C. Fu, J. Am. Chem. Soc. 128 (2006) 5360-5361; D. J. Wallace and C.-Y. Chen, Tetrahedron Letters 43 (2002) 6987-6990; T. E. Barder et al., J. Am. Chem. Soc. 127 (2005) 4685-4696; D. W. Old et al., J. Am. Chem. Soc. 120 (1998) 9722; T. E. Barder and St. L. Buchwald, Org. Lett. 6 (2004) 2649-2652. The further conversion of the thus R2-substituted compound A can be effected as described for method “A” and “B”.

In method “D”, the procedure may also be to react the compound A′ where R2 is halogen, preferably chlorine or bromine, under palladium catalysis, with a diboron compound, e.g. bis(pinacolato)diboron, to give the arylboronate of the formula A″ where R2 is

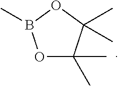

In a further step, this compound can be reacted with an organohalogen compound R2-Hal to give compounds of the formula A in which R2 is, for example, cycloalkyl or aryl. The subsequent reactions to obtain the compounds of the formula H can again be effected according to method “A” or “B”.

Compounds of the formula A in which R2 is —O/S-alkyl, —O/S-cycloalkyl, —O/S—CH₂-aryl, —O/S—CH₂-heteroaryl, —O/S-aryl, —O/S-heteroaryl, can be prepared from compounds of the formula A′ in which R2 is halogen, preferably bromine or chlorine, by reaction with the corresponding alcohols or phenols, or mercaptans or mercaptoaryls and -heteroaryls, and cesium carbonate under palladium or copper catalysis (see also R. Frlan and D. Kikelj; Synthesis 14 (2006) 2271-2285; A. V. Vorogushin et al., J. Am. Chem. Soc. 127 (2005) 8146-8149; F. Y. Kwong and St. L. Buchwald, Org. Lett. 4 (2002) 3517-3520).

Compounds of the formula A in which R2 is —CH₂-aryl or —CH₂-heteroaryl can be obtained, for example, from compounds of the formula A″ by reaction with halomethylaryls or halomethylheteroaryls, where halogen is preferably bromine or chlorine, under basic conditions and palladium catalysis (see also S. M. Nobre and A. L. Monteiro, Tetrahedron Letters 45 (2004) 8225-8228; S. Langle et al., Tetrahedron Letters 44 (2003) 9255-9258).

The processes described here are detailed for the specific case of the urea bridge (—NR23-CO—NR24—). Compounds with other claimed bridges can be prepared correspondingly.

The examples which follow serve to illustrate the invention in detail, without limiting it to the products and embodiments described in the examples.

### General Experimental Methods:

¹H NMR:

The ¹H NMR spectra were measured in deuterated dimethyl sulfoxide on a 500 MHz instrument (Bruker DRX 500) or on a 400 MHz instrument (Bruker DRX 400) at 300K. Data: δ in ppm, multiplicity (s for singlet, d for doublet, t for triplet, q for quartet, m for multiplet, x H (number of hydrogen atoms))

HPLC-MS:

The HPLC-MS analyses were carried out on an LCT instrument from Waters. Column: YMC Jshere 33×2 4 μm; gradient [A]: (acetonitrile+0.05% trifluoroacetic acid):(water+0.05% trifluoroacetic acid) 5:95 (0 minutes) to 95:5 (3 minutes); gradient [B]: (acetonitrile+0.05% trifluoroacetic acid):(water+0.05% trifluoroacetic acid) 5:95 (0 minutes) to 95:5 (2.5 minutes) to 95:5 (3.0 minutes); gradient [C]: (acetonitrile+0.05% trifluoroacetic acid):(water+0.05% trifluoroacetic acid) 5:95 (0 minutes) to 95:5 (3.4 minutes) to 95:5 (4.4 minutes); gradient [D]: (acetonitrile+0.05% trifluoroacetic acid):(water+0.05% trifluoroacetic acid) 2:98 (1 minute) to 95:5 (5 minutes) to 95:5 (6.25 minutes); gradient [E]: (acetonitrile+0.05% trifluoroacetic acid):(water+0.05% trifluoroacetic acid) 5:95 (0 minutes) to 5:95 (0.5 minute) to 95:5 (3.5 minutes) to 95:5 (4.0 minutes); gradient [F]: column: YMC Jsphere ODS H80 20×2 mm, 4 μm; (water+0.05% trifluoroacetic acid):(acetonitrile+0.05% trifluoroacetic acid) 96:4 (0 minutes) to 5:95 (2 minutes) to 96:4 (2.4 minutes); detector: Tecan-LCT.

### Chromatographic Purification Methods:

[RP1]: flow rate: 30 ml/min; gradient: acetonitrile/water+0.1% trifluoroacetic acid; 30 min. column: XTerra C18 5 μm 30×100 mm; detection: MS (ESI), UV (DAD).
[RP2]: flow rate: 150 ml/min; gradient: acetonitrile/water+0.1% trifluoroacetic acid; 20 min. column: XTerra C18 10 μm 50×250 mm; detection: MS (ESI), UV (DAD).

### EXAMPLE 1

### 4-(3-{2-[3-(4-Cyano-3-trifluoromethylphenyl)-5,5-dimethyl-2,4-dioxo-imidazolidin-1-ylmethyl]phenyl}ureido)benzenesulfonamide

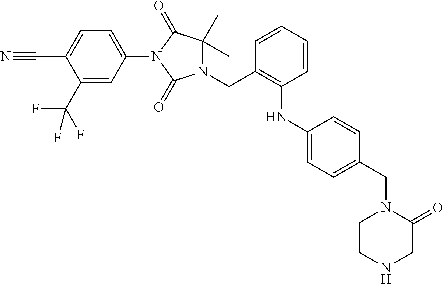

### 1) Preparation of 4-(4,4-dimethyl-2,5-dioxoimidazolidin-1-yl)-2-trifluoromethylbenzonitrile (1.1)

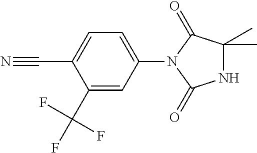 [0495] Compound 1.1 can be prepared by method “A”. To this end, 14.74 g (79.21 mmol) of 4-amino-2-trifluoromethylbenzonitrile were dissolved in 200 ml of dry acetonitrile. This solution was added dropwise with stirring to a 20% solution, heated to 70° C., of phosgene in toluene and then stirred for 1 h. The cooled reaction solution was concentrated under reduced pressure, the residue was taken up with toluene and concentrated again under reduced pressure. Finally, the residue was dissolved in 150 ml of dry acetonitrile and the solution was admixed with stirring with 15.5 g (79.21 mmol) of tert-butyl 2-amino-2-methylpropionate hydrochloride. 12.02 g (118.8 mmol) of triethylamine were slowly added dropwise to the reaction mixture which was then stirred at room temperature for 45 min. Thereafter, the mixture was admixed cautiously with 50 ml of concentrated hydrochloric acid and stirred at 70° C. for 1 h. The cooled reaction mixture was concentrated under reduced pressure and the residue was admixed with ethyl acetate and water. The organic phase was removed, washed with saturated sodium hydrogencarbonate solution and then with water, dried over sodium sulfate, filtered and concentrated under reduced pressure. The residue was purified chromatographically using silica gel with 2:1 heptane/ethyl acetate. This afforded 21.2 g (90% yield) of 444,4-dimethyl-2,5-dioxoimidazolidin-1-yl)-2-trifluoromethylbenzonitrile 1.1 with melting point 208-211° C.

### 2) Preparation of 4-[3-(2-bromobenzyl)-4,4-dimethyl-2,5-dioxoimidazolidin-1-yl]-2-trifluoromethylbenzonitrile (1.2)

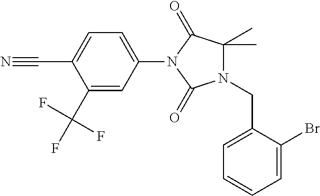 [0497] Compound 1.2 can be prepared by method “A”. To this end, 21.2 g (71.32 mmol) of compound 1.1 and 17.83 g (71.32 mmol) of 2-bromobenzyl bromide were dissolved in 200 ml of dry acetonitrile, admixed with 12.32 g of potassium carbonate and stirred at room temperature for 5 h. For workup, the reaction mixture was admixed with water, the mixture was extracted by shaking with ethyl acetate, and the organic phase was dried over sodium sulfate, filtered and concentrated under reduced pressure. The residue was purified chromatographically using silica gel with 3:1 heptane/ethyl acetate. This afforded 28.5 g (86% yield) of 4-[3-(2-bromobenzyl)-4,4-dimethyl-2,5-dioxoimidazolidin-1-yl]-2-trifluoromethylbenzonitrile (1.2) with melting point 56-58° C.

### 3) Preparation of 4-[3-(2-aminobenzyl)-4,4-dimethyl-2,5-dioxoimidazolidin-1-yl]-2-trifluoromethylbenzonitrile 1.3

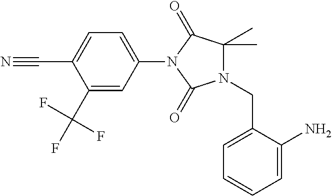 [0499] Compound 1.3 can be prepared by method “A”. 370 mg (0.794 mmol) of the compound of example 1.2 together with 216 mg of benzophenone imine, 776 mg of cesium carbonate, 9 mg of palladium(II) acetate and 46 mg of 9,9-dimethyl-4,5-bis(diphenylphosphino)xanthene were admixed under an argon atmosphere with 2.8 ml of dry dioxane. The mixture was stirred at 95° C. for 6 h; 7.5 ml of 1 N hydrochloric acid were added to the cooled reaction mixture. The mixture was stirred at room temperature for 10 min and neutralized with aqueous sodium hydroxide solution. For workup, the reaction mixture was extracted by shaking 3× with dichloromethane, and the organic phase was dried over magnesium sulfate, filtered and concentrated under reduced pressure. The residue was purified by chromatography (method [RP2]). This afforded 4-[3-(2-aminobenzyl)-4,4-dimethyl-2,5-dioxoimidazolidin-1-yl]-2-trifluoromethylbenzonitrile 1.3 in 78% yield. Molecular weight 402.13 (C₂₀H₁₇F₃N₄O₂); retention time Rₜ=1.61 min. [B]; MS (ESI): 403.06 (MH⁺).

### 4) Preparation of 4-(3-{2-[3-(4-cyano-3-trifluoromethylphenyl)-5,5-dimethyl-2,4-dioxoimidazolidin-1-ylmethyl]phenyl}ureido)benzenesulfonyl chloride 1.4

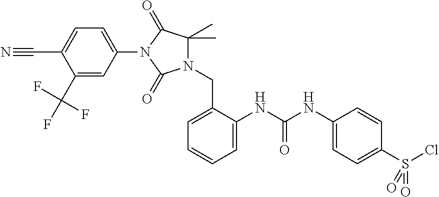 [0501] Compound 1.4 can be prepared by method “A”. To this end, 0.2 g of compound 1.3 was dissolved at room temperature in 5 ml of dry tetrahydrofuran, admixed with 0.11 g of 4-(chlorosulfonyl)phenyl isocyanate, stirred at room temperature for 4 h and left to stand overnight. To complete the conversion, 55 mg of the isocyanate was added and the mixture was stirred at room temperature for a further 6 h. For workup, the reaction mixture was filtered and the filtrate was concentrated under reduced pressure. Chromatographic purification (silica gel; 50/50 n-heptane/ethyl acetate to 33/67 n-heptane/ethyl acetate in 25 min.) afforded 4-(3-{2-[3-(4-cyano-3-trifluoromethylphenyl)-5,5-dimethyl-2,4-dioxoimidazolidin-1-ylmethyl]-phenyl}ureido)benzenesulfonyl chloride 1.4. Molecular weight 619.09 (C₂₇H₂₁ClF₃N₅O₅S); retention time Rₜ=2.52 min. [B]; MS (ES): 618.21 (M-H⁺).

### 5) Preparation of 4-(3-{2-[3-(4-cyano-3-trifluoromethylphenyl)-5,5-dimethyl-2,4-dioxoimidazolidin-1-ylmethyl]phenyl}ureido)benzenesulfonamide 1

0.12 g of compound 1.4 were admixed at room temperature with 3 ml of a solution of ammonia in methanol (7 M) and left to stand at room temperature for 3 days. For workup, the reaction mixture was concentrated under reduced pressure and purified by chromatography (method [RP1]). The eluates were concentrated under reduced pressure, neutralized with a solution of ammonia in water and freeze-dried. This afforded 4-(3-{2-[3-(4-cyano-3-trifluoromethylphenyl)-5,5-dimethyl-2,4-dioxoimidazolidin-1-ylmethyl]phenyl}ureido)benzenesulfonamide 1. Molecular weight 600.14 (C₂₇H₂₃F₃N₆O₅S); retention time Rₜ=1.73 min. [B]; MS (ESI): 601.12 (MH⁺).

Compound 1.3 (4-[3-(2-aminobenzyl)-4,4-dimethyl-2,5-dioxo-imidazolidin-1-yl]-2-trifluoromethylbenzonitrile) can alternatively also be prepared by the following route:

### 1) Preparation of tert-butyl (2-iodomethylphenyl)carbamate 1.3.2

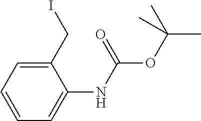 [0505] 0.7 g of imidazole were dissolved at room temperature in 20 ml of dry dichloromethane and admixed with 3.4 g of polymer-bound triphenylphosphine (approx. 3 mmol/g of resin). A solution of 2.5 g of iodine in 80 ml of dry dichloromethane was slowly added dropwise to this mixture with stirring, and the solution was stirred further until discoloration. This mixture was subsequently admixed gradually with a solution of 1.0 g of tert-butyl (2-hydroxymethylphenyl)-carbamate 1.3.1 in 20 ml of dry dichloromethane and stirred at room temperature for 4 h. For workup, the reaction mixture was filtered; the filtrate was washed successively with saturated ammonium chloride solution, water and saturated sodium chloride solution. The organic phase was dried over magnesium sulfate, filtered and concentrated under reduced pressure. The product (tert-butyl (2-iodomethylphenyl)carbamate) was used in the next stage without any further purification.

### 2) Preparation of tert-butyl {2-[3-(4-cyano-3-trifluoromethylphenyl)-5,5-dimethyl-2,4-dioxoimidazolidin-1-ylmethyl]phenyl}carbamate 1.3.3

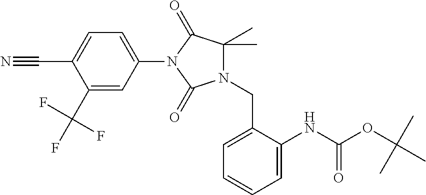 [0507] 0.86 g of compound 1.1 were dissolved at room temperature in 20 ml of dry acetonitrile, admixed with 1.06 g of compound 1.3.2 and 1.04 g of cesium carbonate, stirred at room temperature for 4 h and then left to stand overnight. For workup, the reaction mixture was concentrated under reduced pressure, the residue was taken up with water, the aqueous phase was extracted with dichloromethane, and the organic phase was dried over magnesium sulfate, filtered and concentrated under reduced pressure. The chromatographic purification (silica gel; 67/33 n-heptane/ethyl acetate to 50/50 n-heptane/ethyl acetate in 35 min.) afforded tert-butyl {2-[3-(4-cyano-3-trifluoromethylphenyl)-5,5-dimethyl-2,4-dioxoimidazolidin-1-ylmethyl]-phenyl}carbamate 1.3.3. ¹H NMR: 8.82, s, 1H, 8.35, d, 1H, 8.27, s, 1H, 8.1, d, 1H, 7.44, d, 1H, 7.33, d, 1H, 7.26, t, 1H, 7.13, t, 1H, 4.58, s, 2H, 1.48, s, 9H, 1.35, s, 6H.

### 3) Preparation of 4-[3-(2-aminobenzyl)-4,4-dimethyl-2,5-dioxoimidazolidin-1-yl]-2-trifluoromethylbenzonitrile 1.3

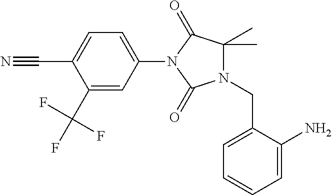 [0509] 1.15 g of compound 1.3.3 were dissolved at room temperature in 20 ml of dry dichloromethane, admixed with 3.5 ml of trifluoroacetic acid and 0.35 ml of water, stirred at room temperature for 4 h and then left to stand overnight. The reaction mixture was concentrated under reduced pressure and the residue was dissolved in toluene and concentrated again under reduced pressure. This residue was dissolved in dichloromethane and washed with saturated sodium hydrogencarbonate solution, and the organic phase was removed, dried over magnesium sulfate, filtered and concentrated under reduced pressure. This afforded 4-[3-(2-aminobenzyl)-4,4-dimethyl-2,5-dioxoimidazolidin-1-yl]-2-trifluoromethylbenzonitrile 1.3. ¹H NMR: 8.34, d, 1H, 8.25, s, 1H, 8.09, d, 1H, 7.2, d, 1H, 7.01, t, 1H, 6.69, d, 1H, 6.57, t, 1H, 5.7, s, broad, 2H, 4.48, s, 2H, 1.39, s, 6H.

### EXAMPLE 2

### 4-(3-{2-[3-(4-cyano-3-trifluoromethylphenyl)-5,5-dimethyl-2,4-dioxoimidazolidin-1-ylmethyl]phenyl}ureido)benzenesulfonic acid

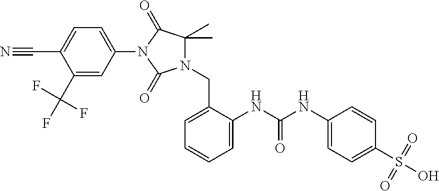 [0511] 0.12 g of the sulfonyl chloride 1.4 was dissolved at room temperature in 2 ml of dry acetonitrile and admixed with 1.9 ml of 1 N sodium hydroxide solution and stirred at room temperature for 2 days. For workup, the reaction mixture was neutralized with 1 N hydrochloric acid and concentrated under reduced pressure. Chromatographic purification (method [RP1]) afforded 4-(3-{2-[3-(4-cyano-3-trifluoromethylphenyl)-5,5-dimethyl-2,4-dioxoimidazolidin-1-yl-methyl]phenyl}ureido)benzenesulfonic acid 2. Molecular weight 601.12 (C₂₇H₂₂F₃N₆O₆S); retention time Rₜ=1.51 min. [B]; MS (ESI): 602.11 (MH⁺).

The compound of example 3, methyl 4-(3-{2-[3-(4-cyano-3-trifluoromethylphenyl)-5,5-dimethyl-2,4-dioxoimidazolidin-1-ylmethyl]phenyl}ureido)benzoate (molecular weight 579.17 (C₂₉H₂₄F₃N₅O₅); retention time Rₜ=1.99 min. [B]; MS (ESI): 580.15 (MH⁺)), of example 5, 1-{2-[3-(4-cyano-3-trifluoromethylphenyl)-5,5-dimethyl-2,4-dioxoimidazolidin-1-ylmethyl]phenyl}-3-(3,4-dimethoxyphenyl)urea (molecular weight 581.18 (C₂₉H₂₆F₃N₅O₅); retention time Rₜ=2.34 min. [C]; MS (ESI): 582.28 (MH⁺)),

of example 6, 1-(4-cyanophenyl)-3-{2-[3-(4-cyano-3-trifluoromethylphenyl)-5,5-dimethyl-2,4-dioxoimidazolidin-1-ylmethyl]phenyl}urea (molecular weight 546.16 (C₂₈H₂₁F₃N₆O₃); retention time Rₜ=1.96 min. [B]; MS (ESI): 547.13 (MH⁺)),
of example 7, 1-{2-[3-(4-cyano-3-trifluoromethylphenyl)-5,5-dimethyl-2,4-dioxoimidazolidin-1-ylmethyl]phenyl}-3-(2,4-difluorophenyl)urea (molecular weight 557.14 (C₂₇H₂₀F₅N₅O₃); retention time Rₜ=2.06 min. [B]; MS (ESI): 558.18 (MH⁺)),
of example 8, 1-(3-cyanophenyl)-3-{2-[3-(4-cyano-3-trifluoromethylphenyl)-5,5-dimethyl-2,4-dioxoimidazolidin-1-ylmethyl]phenyl}urea (molecular weight 546.16 (C₂₈H₂₁F₃N₆O₃); retention time Rₜ=1.96 min. [B]; MS (ESI): 547.15 (MH⁺)),
of example 9, 1-{2-[3-(4-cyano-3-trifluoromethylphenyl)-5,5-dimethyl-2,4-dioxoimidazolidin-1-ylmethyl]phenyl}-3-phenylurea (molecular weight 521.16 (C₂₇H₂₂F₃N₅O₃); retention time Rₜ=1.99 min. [B]; MS (ESI): 522.15 (MH⁺)),
of example 10, 1-{2-[3-(4-cyano-3-trifluoromethylphenyl)-5,5-dimethyl-2,4-dioxoimidazolidin-1-ylmethyl]phenyl}-3-(4-ethoxyphenyl)urea (molecular weight 565.19 (C₂₉H₂₆F₃N₅O₄); retention time Rₜ=2.01 min. [B]; MS (ESI): 566.16 (MH⁺)),
of example 11, methyl 3-(3-{2-[3-(4-cyano-3-trifluoromethylphenyl)-5,5-dimethyl-2,4-dioxoimidazolidin-1-ylmethyl]phenyl}ureido)benzoate (molecular weight 579.17 (C₂₉H₂₄F₃N₅O₅); retention time Rₜ=1.98 min. [B]; MS (ESI): 580.15 (MH⁺)),
of example 35, 1-{2-[3-(4-cyano-3-trifluoromethylphenyl)-5,5-dimethyl-2,4-dioxoimidazolidin-1-ylmethyl]phenyl}-3-(4-methylsulfanylphenyl)urea (molecular weight 567.15 (C₂₈H₂₄F₃N₅O₃S); retention time Rₜ=2.44 min. [B]; MS (ESI): 568.33 (MH⁺)),
were obtained like compound 1.4 by reaction of compound 1.3 with
methyl 4-isocyanatobenzoate (for 3),
4-isocyanato-1,2-dimethoxybenzene (for 5),
4-isocyanatobenzonitrile (for 6),
2,4-difluoro-1-isocyanatobenzene (for 7),
3-isocyanatobenzonitrile (for 8),
isocyanatobenzene (for 9),
1-ethoxy-4-isocyanatobenzene (for 10),
methyl 3-isocyanatobenzoate (for 11),
1-isocyanato-4-methylsulfanylbenzene (for 35).

### EXAMPLE 4

### 4-(3-{2-[3-(4-Cyano-3-trifluoromethylphenyl)-5,5-dimethyl-2,4-dioxo-imidazolidin-1-ylmethyl]phenyl}ureido)benzoic acid

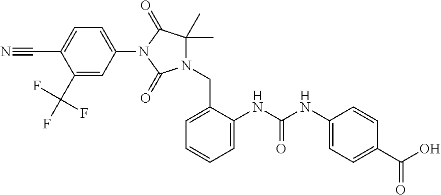 [0523] 0.23 g of the compound of example 3 was dissolved at room temperature in 4 ml of dry dioxane, admixed with 0.33 ml of concentrated hydrochloric acid and stirred at 80° C. for 2 h. To complete the conversion, another 0.67 ml of concentrated hydrochloric acid was added and the mixture was stirred at 80° C. for a further 8 h. For workup, the cooled reaction mixture was concentrated under reduced pressure and the residue was purified by chromatography (method [RP1]). The eluates which contained the product were combined and freeze-dried. This afforded 4-(3-{2-[3-(4-cyano-3-trifluoromethylphenyl)-5,5-dimethyl-2,4-dioxoimidazolidin-1-ylmethyl]phenyl}ureido)benzoic acid 4. Molecular weight 565.15 (C₂₈H₂₂F₃N₅O₅); retention time Rₜ=1.76 min. [B]; MS (ESI): 566.12 (MH⁺).

### EXAMPLE 12

### 1-{2-[3-(4-Cyano-3-trifluoromethylphenyl)-5,5-dimethyl-2,4-dioxo-imidazolidin-1-ylmethyl]phenyl}-3-(4-methane sulfinylphenyl)urea

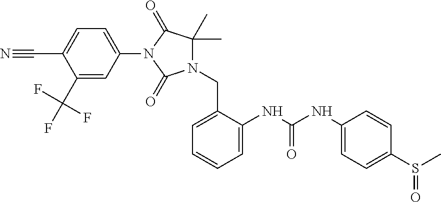 [0525] 0.17 g of sodium periodate were dissolved in 2.5 ml of water. While cooling with an ice bath, a solution of 0.3 g of the compound of example 35 in 5 ml of dry tetrahydrofuran was added slowly and stirred at room temperature for 4 h. The reaction mixture was left to stand at room temperature overnight. For workup, the reaction mixture was admixed with water and then extracted with ethyl acetate. The organic phase was dried over magnesium sulfate, filtered and concentrated under reduced pressure. The crude product was purified by chromatography (method [RP1]). Freeze-drying of the product-containing fractions afforded 1-{2-[3-(4-cyano-3-trifluoromethylphenyl)-5,5-dimethyl-2,4-dioxoimidazolidin-1-ylmethyl]phenyl}-3-(4-methanesulfinylphenyl)urea 12. Molecular weight 583.15 (C₂₈H₂₄F₃N₅O₄S); retention time Rₜ=1.98 min. [C]; MS (ESI): 584.29 (MH⁺).

### EXAMPLE 13

### 1-{2-[3-(4-Cyano-3-trifluoromethylphenyl)-5,5-dimethyl-2,4-dioxoimidazolidin-1-ylmethyl]phenyl}-3-(4-methanesulfonyl-phenyl)ure a

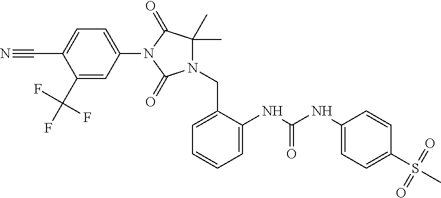 [0527] 0.3 g of the compound of example 35 was dissolved at room temperature in 10 ml of dry dichloromethane, admixed in portions with a total of 0.27 g of m-chloroperbenzoic acid (85%) and stirred at room temperature for 20 h. For workup, the reaction solution was admixed with saturated sodium sulfite solution and stirred briefly. Then the aqueous phase is extracted with dichloromethane; the organic phase is washed with saturated sodium carbonate solution, dried over magnesium sulfate, filtered and concentrated under reduced pressure. The crude product was purified by chromatography (method [RP1]). Freeze-drying of the product-containing fractions afforded 1-{2-[3-(4-cyano-3-trifluoromethylphenyl)-5,5-dimethyl-2,4-dioxoimidazolidin-1-ylmethyl]phenyl}-3-(4-methanesulfonylphenyl)urea 13. Molecular weight 599.14 (C₂₈H₂₄F₃N₅O₅S); retention time Rₜ=2.25 min. [C]; MS (ESI): 600.31 (MH⁺).

### EXAMPLE 16

### 4-(3-{2-[3-(4-Cyano-3-trifluoromethylphenyl)-5,5-dimethyl-2,4-dioxoimidazolidin-1-ylmethyl]phenyl}ureido)benzamide

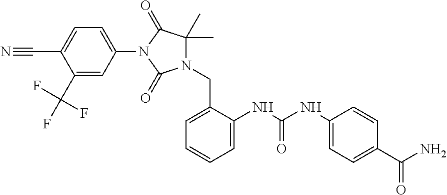 [0529] 0.3 g of the methyl ester of example 3 was admixed at room temperature with 7.4 ml of a 7 M solution of ammonia in methanol and left to stand at room temperature for 6 days. Thereafter, a further 5 ml of the methanolic ammonia solution were added and the mixture was left alone for 10 weeks. For workup, the reaction mixture was concentrated under reduced pressure and the crude product purified by chromatography (method [RP1]). Freeze-drying of the product-containing fractions afforded 4-(3-{2-[3-(4-cyano-3-trifluoromethylphenyl)-5,5-dimethyl-2,4-dioxoimidazolidin-1-ylmethyl]phenyl}ureido)benzamide 16. Molecular weight 564.17 (C₂₈H₂₃F₃N₆O₄); retention time Rₜ=1.68 min. [B]; MS (ESI): 565.26 (MH⁺).

### EXAMPLE 14

### N-{2-[3-(4-Cyano-3-trifluoromethylphenyl)-5,5-dimethyl-2,4-dioxoimidazo lidin-1-ylmethyl]phenyl}-C-(4-trifluoromethylphenyl)-methanesulfonamide

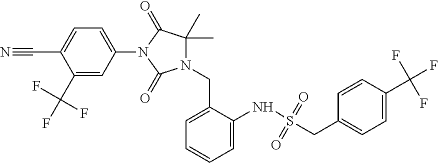 [0531] 0.2 g of compound 1.3 was dissolved in 0 ml of dry dichloromethane, admixed with 60 μl of pyridine and 0.19 g of (4-trifluoromethylphenyl)methanesulfonyl chloride, stirred at room temperature for 4 h and left to stand overnight. For workup, the reaction mixture was concentrated under reduced pressure and the residue was purified by chromatography (method [RP1]). Freeze-drying of the product-containing fractions afforded N-{2-[3-(4-cyano-3-trifluoromethylphenyl)-5,5-dimethyl-2,4-dioxoimidazolidin-1-ylmethyl]phenyl}-C-(4-trifluoromethylphenyl)methanesulfonamide 14. Molecular weight 624.12 (C₂₈H₂₂F₆N₄O₄S); retention time Rₜ=2.55 min. [B]; MS (ES): 623.06 (M-H⁺).

### EXAMPLE 15

### N-{2-[3-(4-Cyano-3-trifluoromethylphenyl)-5,5-dimethyl-2,4-dioxoimidazolidin-1-ylmethyl]phenyl}-C-(2,4-difluorophenyl)-methanesulfonamide

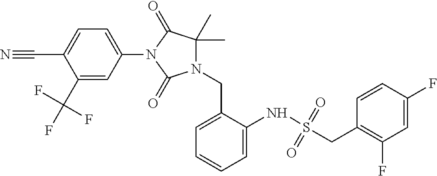 [0533] The compound of example 15 was prepared in an analogous manner by reaction of 1.3 with (2,4-difluorophenyl)methanesulfonyl chloride. This afforded N-{2-[3-(4-cyano-3-trifluoromethylphenyl)-5,5-dimethyl-2,4-dioxoimidazolidin-1-ylmethyl]phenyl}-C-(2,4-difluorophenyl)methanesulfonamide 15. Molecular weight 592.12 (C₂₇H₂₁F₅N₄O₄S); retention time Rₜ=2.06 min. [B]; MS (ESI): 593.15 (MH⁺).

### EXAMPLE 17

### Methyl 4-(3-{2-[3-(4-cyano-3-trifluoromethylphenyl)-5,5-dimethyl-2,4-dioxoimidazolidin-1-ylmethyl]-5-fluorophenyl}ureido)benzoate

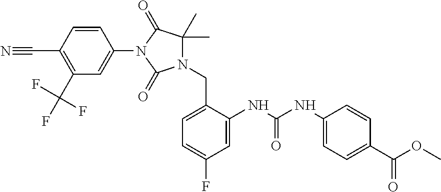

### 1) Preparation of 4-[3-(2-bromo-4-fluorobenzyl)-4,4-dimethyl-2,5-dioxoimidazolidin-1-yl]-2-trifluoromethylbenzonitrile (17.2)

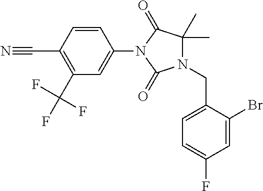 [0536] Compound 17.2 was prepared as described for example 1.2, by reacting compound 1.1, instead of with 2-bromobenzyl bromide, with 2-bromo-1-bromomethyl-4-fluorobenzene (prepared by free-radical bromination from 2-bromo-4-fluoro-1-methylbenzene with N-bromosuccinimide in tetrachloromethane; ¹H NMR: 7.7, m, 1H, 7.65, d, 1H, 7.3, m, 1H, 4.75, s, 2H). Molecular weight 483.02 (C₂₀H₁₄BrF₄N₃O₃); ¹H NMR: 8.37, d, 1H, 8.25, s, 1H, 8.1, d, 1H, 7.65, m, 2H, 7.27, m, 1H, 4.62, s, 2H, 1.41, s, 6H.

### 2) Preparation of 4-[3-(2-amino-4-fluorobenzyl)-4,4-dimethyl-2,5-dioxoimidazolidin-1-yl]-2-trifluoromethylbenzonitrile (17.3)

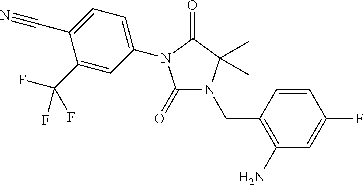 [0538] Analogously to the procedure as described for the preparation of 1.3, 17.2 was reacted with benzophenone imine to give 17.3. Molecular weight 420.12 (C₂₀H₁₆F₄N₄O₂); retention time Rₜ=2.71 min. [E]; MS (ESI): 421.08 (MH⁺).

### 3) Preparation of methyl 4-(3-{2-[3-(4-cyano-3-trifluoromethylphenyl)-5,5-dimethyl-2,4-dioxoimidazolidin-1-ylmethyl]-5-fluorophenyl}ureido)benzoate 17

Compound 17 was prepared analogously to the procedure in the preparation of the compound of example 3, by reacting the aniline 17.3 with methyl 4-isocyanatobenzoate. This afforded methyl 4-(3-{2-[3-(4-cyano-3-trifluoromethylphenyl)-5,5-dimethyl-2,4-dioxoimidazolidin-1-ylmethyl]-5-fluorophenyl}ureido)benzoate. Molecular weight 597.16 (C₂₉H₂₃F₄N₅O₅); retention time Rₜ=2.05 min. [B]; MS (ESI): 598.20 (MH⁺).

The compound of example 36, 1-{2-[3-(4-cyano-3-trifluoromethylphenyl)-5,5-dimethyl-2,4-dioxoimidazolidin-1-ylmethyl]-5-fluorophenyl}-3-(4-methylsulfanylphenyl)urea (molecular weight 585.14 (C₂₈H₂₃F₄N₅O₃S); retention time Rₜ=2.15 min. [B]; MS (ESI): 586.18 (MH⁺)),

of example 20, 1-(6-chloropyridin-3-yl)-3-{2-[3-(4-cyano-3-trifluoromethylphenyl)-5,5-dimethyl-2,4-dioxoimidazolidin-1-ylmethyl]-5-fluorophenyl}urea (isolated as the hydrochloride, molecular weight (free base) 574.11 (C₂₆H₁₉ClF₄N₆O₃); retention time Rₜ=2.46 min. [C]; MS (ESI): 575.8 (MH⁺)),
of example 21, 1-{2-[3-(4-cyano-3-trifluoromethylphenyl)-5,5-dimethyl-2,4-dioxoimidazolidin-1-ylmethyl]-5-fluorophenyl}-3-(2,6-dichloropyridin-4-yl)urea (isolated as the hydrochloride, molecular weight (free base) 608.07 (C₂₆H₁₈Cl₂F₄N₆O₃); retention time Rₜ=2.72 min. [C]; MS (ESI): 609.36 (MH⁺)),
were obtained like compound 1.4 by reaction of compound 17.3 with
1-isocyanato-4-methylsulfanylbenzene (for 36),
2-chloro-5-isocyanatopyridine (for 20),
2,6-dichloro-4-isocyanatopyridine (for 21).

### EXAMPLE 18

### 4-(3-{2-[3-(4-Cyano-3-trifluoromethylphenyl)-5,5-dimethyl-2,4-dioxoimidazolidin-1-ylmethyl]-5-fluorophenyl}ureido)benzoic acid

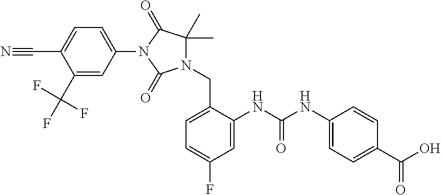 [0545] 0.24 g of the ester of example 17 was dissolved at room temperature in 10 ml of dry tetrahydrofuran, admixed with 0.51 g of potassium trimethylsilanolate, stirred at room temperature for 4 h and then left to stand at room temperature overnight. For workup, the reaction mixture was concentrated under reduced pressure and the residue was purified by chromatography (method [RP1]). This afforded 4-(3-{2-[3-(4-cyano-3-trifluoromethylphenyl)-5,5-dimethyl-2,4-dioxoimidazolidin-1-yl-methyl]-5-fluorophenyl}ureido) benzoic acid 18. Molecular weight 583.14 (C₂₈H₂₁F₄N₅O₅); retention time Rₜ=2.39 min. [C]; MS (ESI): 584.28 (MH⁺).

### EXAMPLE 19

### 1-{2-[3-(4-Cyano-3-trifluoromethylphenyl)-5,5-dimethyl-2,4-dioxoimidazo lidin-1-ylmethyl]-5-fluorophenyl}-3-(4-methane-sulfonylphenyl)urea

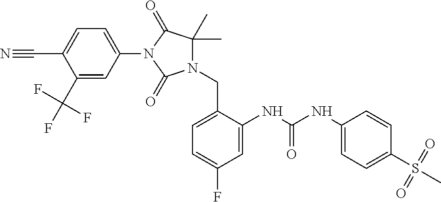 [0547] Analogously to the procedure for the preparation of the compound of example 13, for the preparation of the compound of example 18, compound 36 was oxidized with m-chloroperbenzoic acid. Molecular weight 617.13 (C₂₈H₂₃F₄N₅O₅S); retention time Rₜ=2.28 min. [C]; MS (ESI): 618.27 (MH⁺).

### EXAMPLE 22

### 4-(3-{2-[3-(4-Cyano-3-trifluoromethylphenyl)-5,5-dimethyl-2,4-dioxoimidazolidin-1-ylmethyl]-5-fluorophenyl}ureido)benzenesulfonamide

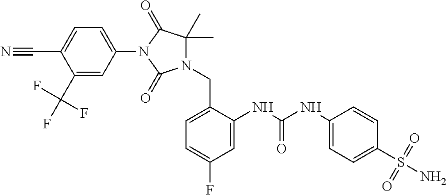

### 1) Preparation of 4-(3-{2-[3-(4-cyano-3-trifluoromethylphenyl)-5,5-dimethyl-2,4-dioxoimidazolidin-1-ylmethyl]-5-fluorophenyl}ureido)benzenesulfonyl chloride (22.1)

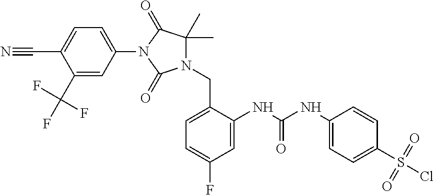 [0550] Analogously to the procedure as described in the preparation of compound 1.4, the aniline 17.3 was reacted with 4-(chlorosulfonyl)phenyl isocyanate and afforded 4-(3-{2-[3-(4-cyano-3-trifluoromethylphenyl)-5,5-dimethyl-2,4-dioxoimidazolidin-1-ylmethyl]-5-fluorophenyl}ureido)benzenesulfonyl chloride (22.1), which was used in the next stage without any further purification.

### 2) Preparation of 4-(3-{2-[3-(4-cyano-3-trifluoromethylphenyl)-5,5-dimethyl-2,4-dioxoimidazolidin-1-ylmethyl]-5-fluorophenyl}ureido)benzenesulfonamide

The reaction of the sulfonyl chloride 22.1 with a solution of ammonia in methanol (see preparation of the compound of example 1) afforded 4-(3-{2-[3-(4-cyano-3-trifluoromethylphenyl)-5,5-dimethyl-2,4-dioxoimidazolidin-1-ylmethyl]-5-fluorophenyl}ureido)benzenesulfonamide (22). Molecular weight 618.13 (C₂₇H₂₂F₄N₆O₅S); retention time Rₜ=2.16 min. [C]; MS (ESI): 619.45 (MH⁺).

### EXAMPLE 23

### 1-(4-Aminophenyl)-3-{5-fluoro-2-[3-(4-fluoro-3-trifluoromethylphenyl)-5,5-dimethyl-2,4-dioxoimidazolidin-1-ylmethyl]phenyl}urea hydrochloride

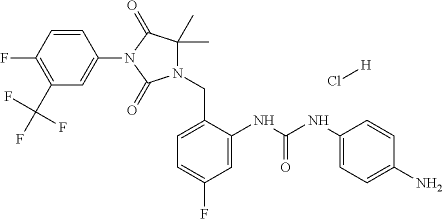

### 1) Preparation of 3-(4-fluoro-3-trifluoromethylphenyl)-5,5-dimethylimidazolidine-2,4-dione (23.1)

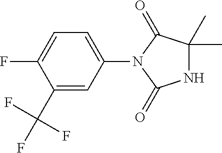 [0554] Compound 23.1 can be prepared by method “A”. To this end, 1.5 g (9.76 mmol) of methyl 2-amino-2-methylpropionate hydrochloride were suspended in 20 ml of dry tetrahydrofuran, and admixed with 1.38 ml (9.76 mmol) of triethylamine and 2 g (9.76 mmol) of 1-fluoro-4-isocyanato-2-trifluoromethylbenzene. The mixture was stirred at 70° C. for 1 h; then the mixture was allowed to cool somewhat, 10 ml of concentrated hydrochloric acid were added and the mixture was stirred at 70° C. for 2 h. The cooled reaction mixture was admixed with ethyl acetate and water; the organic phase was removed, dried over sodium sulfate, filtered and concentrated under reduced pressure. The residue was purified by chromatography (method [RP2]) and, after dissolution in ethyl acetate, drying of the solution, concentration under reduced pressure and redissolution in dichloromethane, crystallized with n-heptane. This afforded 2.8 g of 3-(4-fluoro-3-trifluoromethylphenyl)-5,5-dimethylimidazolidine-2,4-dione (23.1) with melting point 111-114° C. Molecular weight 290.06 (C₁₂H₁₀F₄N₂O₂); retention time Rₜ=1.55 min. [B]; MS (ESI): 291.27 (MH⁺).

### 2) Preparation of 1-(2-bromo-4-fluorobenzyl)-3-(4-fluoro-3-trifluoromethylphenyl)-5,5-dimethylimidazolidine-2,4-dione 23.2

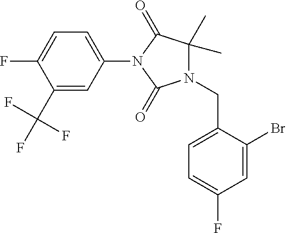 [0556] The title compound was prepared by reacting 23.1 with 2-bromo-1-bromomethyl-4-fluorobenzene under conditions as described above for 1.2. ¹H NMR: 7.98, m, 1H, 7.9, m, 1H, 7.7-7.6, m, 3H, 7.26, m, 1H, 4.6, s, 2H, 1.4, s, 6H.

### 3) Preparation of 1-(2-amino-4-fluorobenzyl)-3-(4-fluoro-3-trifluoromethylphenyl)-5,5-dimethylimidazolidine-2,4-dione 23.3

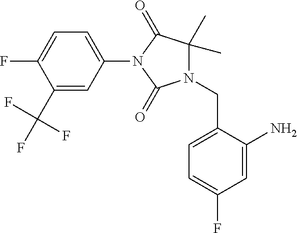 [0558] Analogously to the procedure as described for the preparation of 1.3, 23.2 was reacted with benzophenone imine to give 23.3. The compound was isolated as the salt with trifluoroacetic acid. Molecular weight (free base) 413.11 (C₁₉H₁₆F₅N₃O₂); retention time Rₜ=2.40 min. [C]; MS (ESI): 414.21 (MH⁺).

### 4) Preparation of 1-{5-fluoro-2-[3-(4-fluoro-3-trifluoromethylphenyl)-5,5-dimethyl-2,4-dioxoimidazolidin-1-ylmethyl]phenyl}-3-(4-nitrophenyl)urea 23.4

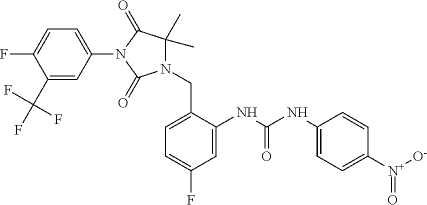 [0560] Analogously to the procedure as described in the preparation of compound 1.4, the aniline 23.3 was reacted with 1-isocyanato-4-nitrobenzene and afforded 1-{5-fluoro-2-[3-(4-fluoro-3-trifluoromethylphenyl)-5,5-dimethyl-2,4-dioxoimidazolidin-1-ylmethyl]phenyl}-3-(4-nitrophenyl)urea (23.4). Molecular weight 577.13 (C₂₆H₂OF5N₅O₅); retention time Rₜ=2.71 min. [C]; MS (ESI): 578.13 (MH¹).

### 5) Preparation of 1-(4-aminophenyl)-3-{5-fluoro-2-[3-(4-fluoro-3-trifluoromethylphenyl)-5,5-dimethyl-2,4-dioxoimidazolidin-1-ylmethyl]phenyl}urea hydrochloride

0.22 g of the compound of example 23.4 was dissolved at room temperature in 3 ml of dry methanol, diluted with 1.5 ml of formic acid, admixed with 45 mg of Raney nickel under argon and stirred at room temperature for 6 h. The reaction mixture was left to stand at room temperature for 2 days. For workup, the mixture was filtered through a depth filter and the filtrate was concentrated under reduced pressure. Chromatographic purification (method [RP1]) afforded the trifluoroacetic acid salt of the compound dissolved in a mixture of water with acetonitrile. The solvent mixture was removed under reduced pressure, and the residue was admixed with a solution of hydrochloric acid in dioxane and then freeze-dried. This afforded 1-(4-aminophenyl)-3-{5-fluoro-2-[3-(4-fluoro-3-trifluoromethylphenyl)-5,5-dimethyl-2,4-dioxoimidazolidin-1-ylmethyl]phenyl}urea hydrochloride (23). Molecular weight (free base) 547.16 (C₂₆H₂₂F₅N₅O₃); retention time Rₜ=1.50 min. [B]; MS (ESI): 548.11 (MH⁺).

### Alternative preparation of 1-(2-amino-4-fluorobenzyl)-3-(4-fluoro-3-trifluoromethylphenyl)-5,5-dimethylimidazolidine-2,4-dione 23.3

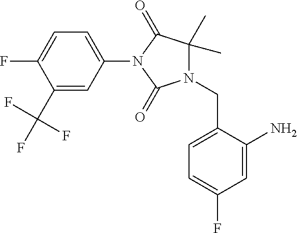

### 1) Preparation of 1-bromomethyl-4-fluoro-2-nitrobenzene 23.10

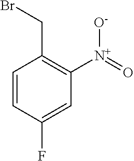 [0564] 1.7 g of 4-fluoronitrobenzyl alcohol were dissolved at room temperature in 30 ml of dry dichloromethane and admixed dropwise at room temperature with a solution of 0.38 ml of phosphorus tribromide in 10 ml of dry dichloromethane. After the addition had ended, the reaction mixture was stirred overnight at room temperature. For workup, the mixture was admixed with saturated sodium hydrogencarbonate solution and extracted with dichloromethane. The organic phase was washed with saturated sodium chloride solution, dried over magnesium sulfate, filtered and concentrated under reduced pressure. This afforded 1-bromomethyl-4-fluoro-2-nitro-benzene. ¹H NMR: 8.02, d, 1H, 7.85, m, 1H, 7.69, t, 1H, 4.91, s, 2H.

### 2) Preparation of 1-(4-fluoro-2-nitrobenzyl)-3-(4-fluoro-3-trifluoromethylphenyl)-5,5-dimethylimidazolidine-2,4-dione 23.11

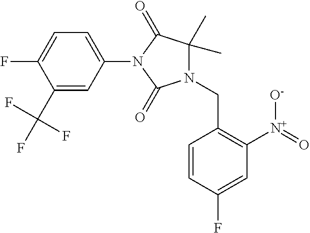 [0566] The reaction of 23.1 with 23.10 in acetonitrile with cesium carbonate afforded 1-(4-fluoro-2-nitrobenzyl)-3-(4-fluoro-3-trifluoromethylphenyl)-5,5-dimethylimidazolidine-2,4-dione 23.11. Molecular weight 443.09 (C₁₉H₁₄F₅N₃O₄); retention time Rₜ=2.82 min. [E]; MS (ESI): 444.12 (MH⁺).

### 3) Preparation of 1-(2-amino-4-fluorobenzyl)-3-(4-fluoro-3-trifluoromethylphenyl)-5,5-dimethylimidazolidine-2,4-dione 23.3

2.33 g of compound 23.11 were dissolved at room temperature in 50 ml of dry methanol and admixed under argon with 37 mg of palladium hydroxide on carbon. Thereafter, 0.46 g of trimethylamine-borane was added and the mixture was stirred under reflux for 4 h. For workup, the reaction mixture was filtered, the filtrate was concentrated under reduced pressure and the residue was taken up with diisopropyl ether. The suspension was filtered, the filtrate concentrated under reduced pressure and the residue freeze-dried. This afforded 1-(2-amino-4-fluorobenzyl)-3-(4-fluoro-3-trifluoromethylphenyl)-5,5-dimethylimidazolidine-2,4-dione (23.3). ¹H NMR: 7.98, d, 1H, 7.88, m, 1H, 7.7, t, 1H, 7.21, m, 1H, 6.44, m, 1H, 6.31, m, 1H, 5.45, s, broad, 2H, 4.42, s, 2H, 1.38, s, 6H.

### EXAMPLE 24

### 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonamide

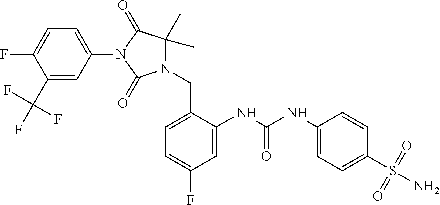

### 1) Preparation of 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonyl chloride (24.1)

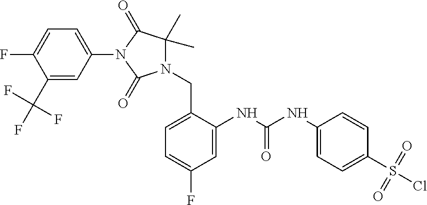 [0570] Analogously to the procedure as described in the preparation of compound 1.4, the aniline 23.3 was reacted with 4-(chlorosulfonyl)phenyl isocyanate and afforded 4-(3-{5-fluoro-2-[3-(4-fluoro-3-trifluoromethylphenyl)-5,5-dimethyl-2,4-dioxoimidazo lidin-1-ylmethyl]phenyl}ureido)benzenesulfonyl chloride (24.1), which was used in the next stage without any further purification.

### 2) Preparation of 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonamide

The reaction of the sulfonyl chloride 24.1 with a solution of ammonia in methanol (see preparation of the compound of example 1) afforded 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonamide (24). Molecular weight 611.12 (C₂₆H₂₂F₅N₅O₅S); retention time Rₜ=1.83 min. [B]; MS (ESI): 612.30 (MH⁺).

### EXAMPLE 25

### 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonic acid

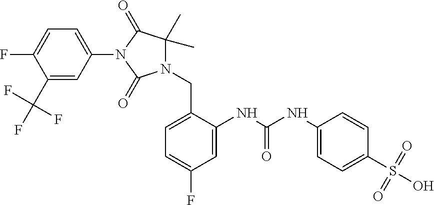 [0573] 0.4 g of the sulfonyl chloride 24.1 was suspended at room temperature in 3 ml of water, admixed with 0.65 ml of pyridine and stirred at 80° C. for 4 h. The reaction mixture was concentrated under reduced pressure and purified by chromatography (method [RP1]). The product-containing fractions were freeze-dried and afforded 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonic acid 25. Molecular weight 612.11 (C₂₆H₂₁F₅N₄O₆S); retention time Rₜ=1.57 min. [B]; MS (ESI): 613.28 (MH⁺).

### EXAMPLE 26

### Sodium salt of 4-(3-{5-fluoro-2-[3-(4-fluoro-3-trifluoromethylphenyl)-5,5-dimethyl-2,4-dioxoimidazolidin-1-ylmethyl]phenyl}ureido)benzenesulfonic acid

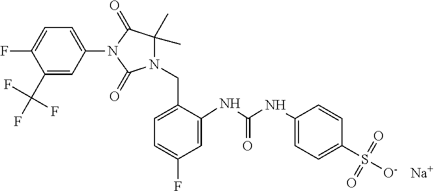 [0575] 0.32 g of the sulfonic acid of example 25 was dissolved at room temperature in 20 ml of water and admixed with sodium hydrogencarbonate until a pH of approx. 7 had been attained. The solution was freeze-dried and afforded the sodium salt 26. ¹H NMR: 9.2, s, 1H, 8.45, s, 1H, 8.0, m, 1H, 7.88, m, 1H, 7.65, m, 2H, 7.5, d, 2H, 7.44, m, 3H, 6.9, t, 1H, 4.58, s, 2H, 1.4, s, 6H.

### EXAMPLE 27

### 1-{5-Fluoro-2-[3-(4-fluoro-3-trifluoromethylphenyl)-5,5-dimethyl-2,4-dioxoimidazolidin-1-ylmethyl]phenyl}-3-(4-methanesulfonylphenyl)urea

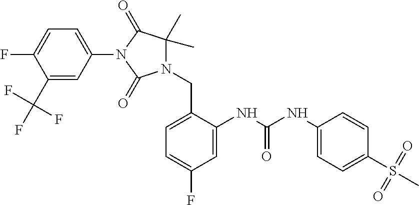

### 1) Preparation of 1-{5-fluoro-2-[3-(4-fluoro-3-trifluoromethylphenyl)-5,5-dimethyl-2,4-dioxoimidazolidin-1-ylmethyl]phenyl}-3-(4-methylsulfanylphenyl)urea (example 37)

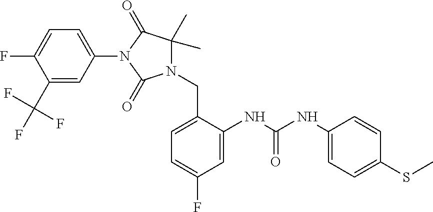 [0578] The compound of example 37, 1-{5-fluoro-2-[3-(4-fluoro-3-trifluoromethylphenyl)-5,5-dimethyl-2,4-dioxoimidazo lidin-1-ylmethyl]phenyl}-3-(4-methylsulfanylphenyl)urea (molecular weight 578.14 (C₂₇H₂₃F₅N₄O₃S); retention time Rₜ=2.22 min. [B]; MS (ESI): 579.29 (MH⁺)), was obtained like compound 1.4 by reaction of compound 23.3 with 1-isocyanato-4-methylsulfanylbenzene.

### 2) Preparation of 1-{5-fluoro-2-[3-(4-fluoro-3-trifluoromethylphenyl)-5,5-dimethyl-2,4-dioxoimidazolidin-1-ylmethyl]phenyl}-3-(4-methanesulfonylphenyl)urea

Analogously to the procedure for the preparation of the compound of example 13, for the preparation of the compound of example 27, compound 37 was oxidized with m-chloroperbenzoic acid. Molecular weight 610.13 (C₂₇H₂₃F₅N₄O₅S); retention time Rₜ=1.91 min. [B]; MS (ESI): 611.30 (MH⁺).

### EXAMPLE 28

### 4-(3-{2-[3-(4-Cyano-3-cyclopropylphenyl)-5,5-dimethyl-2,4-dioxoimidazolidin-1-ylmethyl]-5-fluorophenyl}ureido)benzenesulfonamide

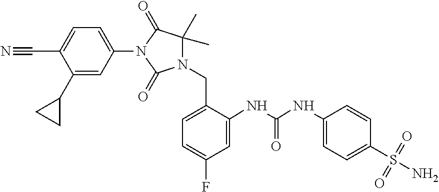

### 1) Preparation of 4-amino-2-cyclopropylbenzonitrile (28.5)

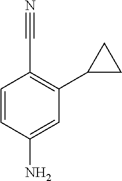 [0582] To prepare the compound of example 28, method “D” is employed: To a suspension of 916 mg of 2-chloro-4-aminobenzonitrile, 773 mg of cyclopropylboronic acid, 5.094 g of potassium phosphate and 673 mg of tricyclohexylphosphine in a mixture of 10.5 ml of toluene and 1.74 ml of water were added, under an argon atmosphere, 269 mg of palladium acetate. The mixture was stirred at 80° C. overnight. The cooled reaction mixture was admixed with water and ethyl acetate and filtered, and the filtrate was extracted three times with a mixture of ethyl acetate with toluene. The organic phases were dried over magnesium sulfate, filtered and concentrated under reduced pressure. The residue was purified by chromatography (method [PR1]) and afforded 4-amino-2-cyclopropylbenzonitrile. ¹H NMR: 7.3, d, 1H, 6.4, d, 1H, 6.1, d, 1H, 4.0, s (broad), 2H, 2.0, m, 1H, 1.0, m, 2H, 0.65, m, 2H.

### 2) Preparation of 2-cyclopropyl-4-(4,4-dimethyl-2,5-dioxoimidazolidin-1-yl)-benzonitrile (28.1)

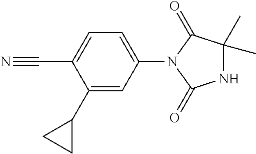 [0584] Compound 28.1 was prepared by the process as described for 1.1 by reaction of 28.5 with tert-butyl 2-amino-2-methylpropionate hydrochloride and phosgene (solution in toluene). Molecular weight 269.11 (C₁₅H₁₅N₃O₂); retention time Rₜ=1.43 min. [B]; MS (ESI): 270.18 (MH⁺).

### 3) Preparation of 4-[3-(2-bromo-4-fluorobenzyl)-4,4-dimethyl-2,5-dioxoimidazolidin-1-yl]-2-cyclopropylbenzonitrile (28.2)

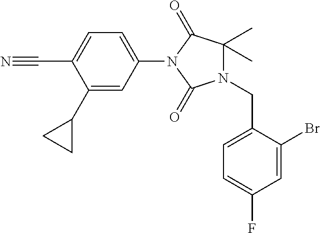 [0586] Under conditions as described for the preparation of 1.2, compound 28.1 was reacted with 2-bromo-1-bromomethyl-4-fluorobenzene and afforded 28.2. Molecular weight 455.06 (C₂₂H₁₉BrFN₃O₂); retention time Rₜ=2.15 min. [B]; MS (ESI): 456.02 (MH⁺).

### 4) Preparation of 4-[3-(2-amino-4-fluorobenzyl)-4,4-dimethyl-2,5-dioxoimidazolidin-1-yl]-2-cyclopropylbenzonitrile 28.3

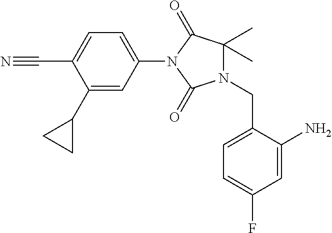 [0588] Analogously to the procedure as described for the preparation of 1.3, 28.2 was reacted with benzophenone imine to give 28.3. The compound was isolated as the salt with trifluoroacetic acid. Molecular weight (free base) 392.16 (C₂₂H₂₁FN₄O₂); retention time Rₜ=1.87 min. [B]; MS (ESI): 393.25 (MH⁺).

### 5) Preparation of 4-(3-{2-[3-(4-cyano-3-cyclopropylphenyl)-5,5-dimethyl-2,4-dioxoimidazolidin-1-ylmethyl]-5-fluorophenyl}ureido)benzenesulfonyl chloride 28.4

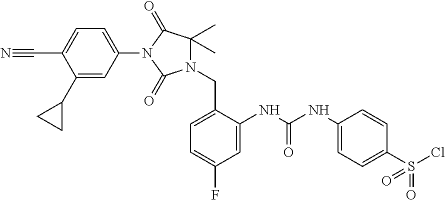 [0590] Analogously to the procedure as described in the preparation of compound 1.4, the aniline 28.3 was reacted with 4-(chlorosulfonyl)phenyl isocyanate and afforded 443-{2-[3-(4-cyano-3-cyclopropylphenyl)-5,5-dimethyl-2,4-dioxoimidazo lidin-1-ylmethyl]-5-fluorophenyl}ureido)benzenesulfonyl chloride (28.4), which was used in the next stage without any further purification.

### 6) Preparation of 4-(3-{2-[3-(4-cyano-3-cyclopropylphenyl)-5,5-dimethyl-2,4-dioxoimidazolidin-1-ylmethyl]-5-fluorophenyl}ureido)benzenesulfonamide 28

The reaction of the sulfonyl chloride 28.4 with a solution of ammonia in methanol (see preparation of the compound of example 1) afforded 4-(3-{2-[3-(4-cyano-3-cyclopropylphenyl)-5,5-dimethyl-2,4-dioxoimidazolidin-1-ylmethyl]-5-fluorophenyl}ureido)benzenesulfonamide (28). Molecular weight 590.17 (C₂₉H₂₇FN₆O₅S); retention time Rₜ=1.76 min. [B]; MS (ESI): 591.32 (MH⁺).

### EXAMPLE 29

### 4-(3-{2-[3-(4-cyano-3-cyclopropylphenyl)-5,5-dimethyl-2,4-dioxo-imidazolidin-1-ylmethyl]-5-fluorophenyl}ureido)benzenesulfonic acid

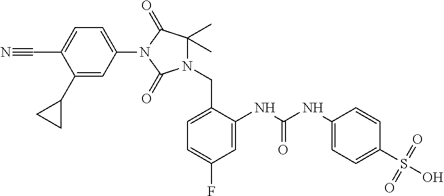 [0593] Analogously to the procedure for the preparation of the compound of example 25, the sulfonyl chloride 28.4 was reacted with pyridine and afforded, after chromatographic purification, 4-(3-{2-[3-(4-cyano-3-cyclopropylphenyl)-5,5-dimethyl-2,4-dioxoimidazolidin-1-ylmethyl]-5-fluorophenyl}ureido)benzenesulfonic acid (29). Molecular weight 591.15 (C₂₉H₂₆FN₅O₆S); retention time Rₜ=1.51 min. [B]; MS (ESI): 592.30 (MH⁺).

### EXAMPLE 30

### 1-{2-[3-(4-Cyano-3-cyclopropylphenyl)-5,5-dimethyl-2,4-dioxoimidazolidin-1-ylmethyl]-5-fluorophenyl}-3-(4-methanesulfonylphenyl)urea

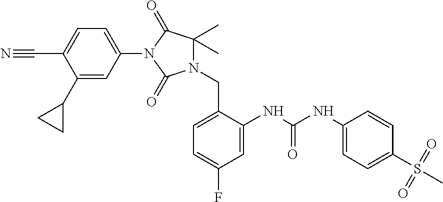

### 1) Preparation of 1-{2-[3-(4-cyano-3-cyclopropylphenyl)-5,5-dimethyl-2,4-dioxoimidazolidin-1-ylmethyl]-5-fluorophenyl}-3-(4-methylsulfanylphenyl)urea (30.1)

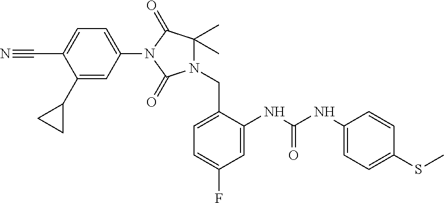 [0596] Compound 30.1, 1-{2-[3-(4-cyano-3-cyclopropylphenyl)-5,5-dimethyl-2,4-dioxoimidazolidin-1-ylmethyl]-5-fluorophenyl}-3-(4-methylsulfanylphenyl)urea, was obtained as described for compound 35 by reaction of compound 28.3 with 1-isocyanato-4-methylsulfanylbenzene. Molecular weight 557.18 (C₃₀H₂₈FN₅O₃S); retention time Rₜ=3.70 min. [E]; MS (ESI): 558.14 (MH⁺).

### 2) Preparation of 1-{2-[3-(4-cyano-3-cyclopropylphenyl)-5,5-dimethyl-2,4-dioxoimidazo lidin-1-ylmethyl]-5-fluorophenyl}-3-(4-methanesulfonylphenyl)urea

Analogously to the procedure for the preparation of the compound of example 13, for the preparation of the compound of example 30, compound 30.1 was oxidized with m-chloroperbenzoic acid. Molecular weight 589.17 (C₃₀H₂₈FN₅O₅S); retention time Rₜ=1.86 min. [B]; MS (ESI): 590.31 (MH⁺).

### EXAMPLE 31

### N-[4-(3-{5-Fluoro-2-[3-(4-fluoro-3-trifluoromethylphenyl)-5,5-dimethyl-2,4-dioxoimidazolidin-1-ylmethyl]phenyl}ureido)phenyl]methanesulfonamide

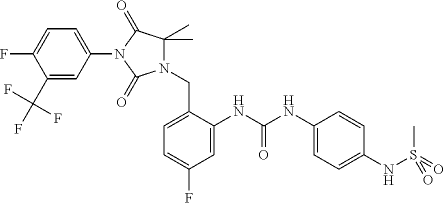 [0599] 0.21 g of the compound of example 23 was dissolved in 5 ml of dry dichloromethane, admixed with 46 μl of pyridine and 44 μl of methanesulfonyl chloride and stirred at room temperature for 4 h. For workup, the reaction mixture was concentrated under reduced pressure and the residue was purified by chromatography (method [RP1]). The freeze-drying of the product-containing eluates gave N-[4-(3-{5-fluoro-2-[3-(4-fluoro-3-trifluoromethylphenyl)-5,5-dimethyl-2,4-dioxoimidazolidin-1-ylmethyl]phenyl}ureido)phenyl]methanesulfonamide 31. Molecular weight 625.14 (C₂₇H₂₄F₅N₅O₅S); retention time Rₜ=1.88 min. [B]; MS (ESI): 626.42 (MH⁺).

### EXAMPLE 32

### Diethyl [4-(3-{5-fluoro-2-[3-(4-fluoro-3-trifluoromethylphenyl)-5,5-dimethyl-2,4-dioxoimidazolidin-1-ylmethyl]phenyl}ureido)phenyl]phosphonate

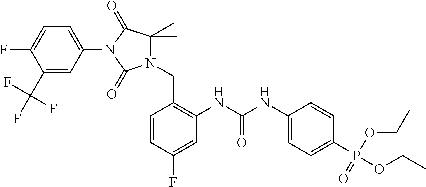 [0601] To 0.74 ml of a solution of phosgene in toluene (20%) was slowly added dropwise, under argon at 75° C., 0.29 g of compound 23.3, dissolved in 10 ml of dry acetonitrile. After the addition had ended, the mixture was stirred at 80° C. for 2 h. Thereafter, the reaction mixture was concentrated under reduced pressure, and the residue was admixed with toluene and concentrated again under reduced pressure. The residue was dissolved in 10 ml of dry tetrahydrofuran and admixed with 0.16 g of diethyl 4-aminophenylphosphonate. To this mixture was slowly added dropwise 0.15 ml of triethylamine. Subsequently, the mixture was stirred at room temperature for 4 h and then left to stand at room temperature overnight. The reaction mixture was concentrated under reduced pressure and the residue was purified by chromatography (method [RP1]). This afforded diethyl [4-(3-{5-fluoro-2-[3-(4-fluoro-3-trifluoromethylphenyl)-5,5-dimethyl-2,4-dioxoimidazolidin-1-ylmethyl]phenyl}ureido)phenyl]phosphonate. Molecular weight 668.18 (C₃₀H₃₀F₅N₄O₆P); retention time Rₜ=2.70 min. [E]; MS (ESI): 669.34 (MH⁺).

### EXAMPLE 33

### Monoethyl [4-(3-{5-fluoro-2-[3-(4-fluoro-3-trifluoromethylphenyl)-5,5-dimethyl-2,4-dioxoimidazolidin-1-ylmethyl]phenyl}ureido)phenyl]-phosphonate

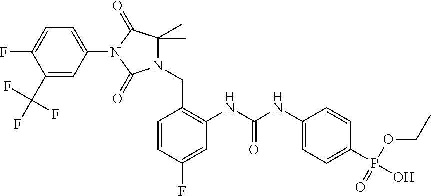 [0603] When the compound of example 32 was treated with potassium trimethylsilanolate in tetrahydrofuran, the monoethyl ester 33 was isolated. Molecular weight 640.15 (C₂₄H₂₆F₅N₄O₆P); retention time Rₜ=2.40 min. [E]; MS (ESI): 641.23 (MH⁺).

### EXAMPLE 34

### [4-(3-{5-Fluoro-2-[3-(4-fluoro-3-trifluoromethylphenyl)-5,5-dimethyl-2,4-dioxoimidazolidin-1-ylmethyl]phenyl}ureido)phenyl]phosphonic acid

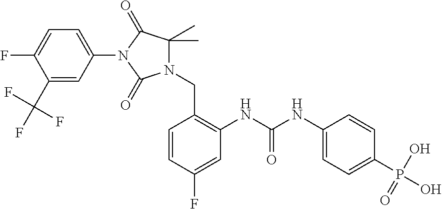 [0605] A solution of the compound of example 33 in dioxane was admixed with concentrated hydrochloric acid and stirred at 80° C. for 8 h. This afforded [4-(3-{5-fluoro-2-[3-(4-fluoro-3-trifluoromethylphenyl)-5,5-dimethyl-2,4-dioxoimidazolidin-1-ylmethyl]phenyl}ureido)phenyl]phosphonic acid 34. Molecular weight 612.11 (C₂₆H₂₂F₅N₄O₆P); retention time Rₜ=2.26 min. [E]; MS (ESI): 613.18 (MH¹).

### EXAMPLE 38

### 1-{4-[5,5-Dimethyl-2,4-dioxo-3-(2-phenoxyphenyl)imidazolidin-1-ylmethyl]phenyl}-3-phenylurea

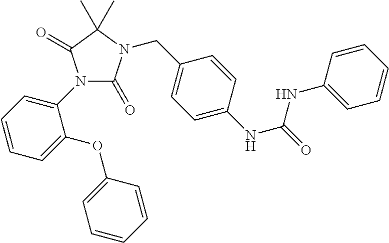

### 1) Preparation of 5,5-dimethyl-3-(2-phenoxyphenyl)imidazolidine-2,4-dione (38.1)

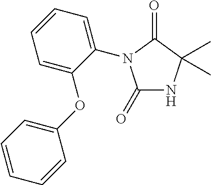 [0608] Compound 38.1, 5,5-dimethyl-3-(2-phenoxyphenyl)imidazolidine-2,4-dione, was obtained analogously to the manner described for compound 23.1, by reaction of tert-butyl 2-amino-2-methylpropionate hydrochloride with triethylamine and 1-isocyanato-2-phenoxybenzene. ¹H NMR: 8.4, s, 1H, 7.5-7.25, m, 5H, 7.18, m, 2H, 6.9, d, 2H, 1.35, s, 3H, 1.1, s, 3H.

### 2) Preparation of 1-{4-[5,5-dimethyl-2,4-dioxo-3-(2-phenoxyphenyl)imidazolidin-1-ylmethyl]phenyl}-3-phenylurea

The alkylating reaction of 38.1 with 1-(4-chloromethylphenyl)-3-phenylurea with cesium carbonate in acetonitrile afforded the title compound of example 38. Molecular weight 520.21 (C₃₁H₂₈N₄O₄); retention time Rₜ=2.41 min. [C]; MS (ESI): 521.19 (MH⁺).

In a corresponding manner, the compound of example 39

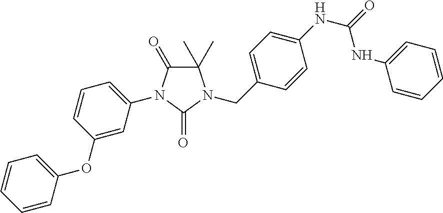

(1-{4-[5,5-dimethyl-2,4-dioxo-3-(3-phenoxyphenyl)imidazolidin-1-ylmethyl]phenyl}-3-phenylurea; molecular weight 520.21 (C₃₁H₂₈N₄O₄); retention time Rₜ=2.06 min. [B]; MS (ESI): 521.24 (MH⁺)) by alkylation of 39.1 (5,5-dimethyl-3-(3-phenoxyphenyl)imidazolidine-2,4-dione; prepared by reaction of tert-butyl 2-amino-2-methylpropionate with 1-isocyanato-3-phenoxybenzene; ¹H NMR: 8.55, s, 1H, 7.5-7.38, m, 3H, 7.16, m, 2H, 7.1-7.0, m, 4H, 1.39, s, 6H) with 1-(4-chloromethylphenyl)-3-phenylurea.

In analogy, the compound of example 40

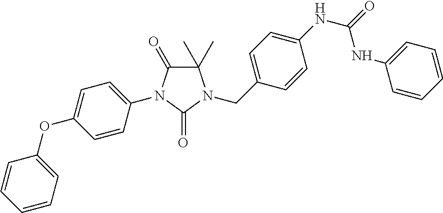

(1-{4-[5,5-dimethyl-2,4-dioxo-3-(4-phenoxyphenyl)-imidazolidin-1-ylmethyl]phenyl}-3-phenylurea; molecular weight 520.21 (C₃₁H₂₈N₄O₄); retention time Rₜ=2.04 min. [B]; MS (ESI): 521.52 (MH⁺)) was also obtained by alkylation of 40.1 (5,5-dimethyl-3-(4-phenoxyphenyl)imidazolidine-2,4-dione; prepared by reaction of tert-butyl 2-amino-2-methylpropionate with 1-isocyanato-4-phenoxybenzene; ¹H NMR: 8.52, s, 1H, 7.47-7.35, m, 4H, 7.20, t, 1H, 7.08, m, 4H, 1.40, s, 6H) with 1-(4-chloromethylphenyl)-3-phenylurea.

### EXAMPLE 41

### 1-{2-[3-(4-Cyano-3-trifluoromethylphenyl)-5,5-dimethyl-2,4-dioxo-imidazolidin-1-ylmethyl]-5-fluorophenyl}-3-(4-hydroxyphenyl)urea

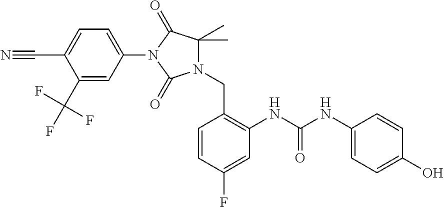 [0613] To 0.38 ml of a solution of phosgene in toluene (20%) was slowly added dropwise, under argon at 75° C., 0.15 g of compound 17.3, dissolved in 10 ml of dry acetonitrile. After the addition had ended, the mixture was stirred at 80° C. for 2 h. Thereafter, the reaction mixture was concentrated under reduced pressure, and the residue was admixed with toluene and concentrated again under reduced pressure. The residue was dissolved in 5 ml of dry pyridine and admixed with 59 mg of p-aminophenol. Subsequently, the mixture was stirred at room temperature for 4 h and then left to stand at room temperature overnight. The reaction mixture was concentrated under reduced pressure and the residue was purified by chromatography (method [RP1]). After freeze-drying, 1-{2-[3-(4-cyano-3-trifluoromethylphenyl)-5,5-dimethyl-2,4-dioxoimidazolidin-1-ylmethyl]-5-fluorophenyl}-3-(4-hydroxyphenyl)urea was obtained. Molecular weight 555.15 (C₂₇H₂₁F₄N₅O₄); retention time Rₜ=3.22 min. [E]; MS (ESI): 556.12 (MH⁺).

The compound of example 42 (isolated as the hydrochloride),

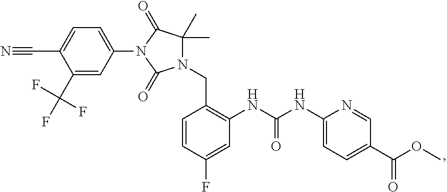

methyl 6-(3-{2-[3-(4-cyano-3-trifluoromethylphenyl)-5,5-dimethyl-2,4-dioxoimidazolidin-1-ylmethyl]-5-fluorophenyl}ureido)nicotinate (molecular weight (free base) 598.15 (C₂₈H₂₂F₄N₆O₅); retention time Rₜ=3.45 min. [E]; MS (ESI): 599.07 (MH⁺)), of example 43,

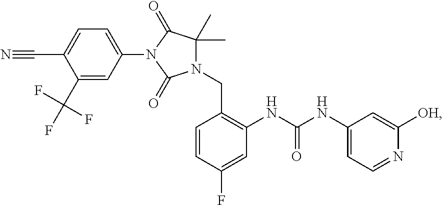

1-{2-[3-(4-cyano-3-trifluoromethylphenyl)-5,5-dimethyl-2,4-dioxoimidazolidin-1-ylmethyl]-5-fluorophenyl}-3-(2-hydroxypyridin-4-yl)urea (isolated as the hydrochloride), molecular weight (free base) 556.14 (C₂₆H₂₀F₄N₆O₄); retention time Rₜ=2.88 min. [E]; MS (ESI): 557.09 (MH⁺)), were obtained like compound 41 by reaction of compound 17.3 with
methyl 6-aminonicotinate (for 42),
3-deazacytosine (for 43).

### EXAMPLE 44

### 1-{5-Fluoro-2-[3-(4-fluoro-3-trifluoromethylphenyl)-5,5-dimethyl-2,4-dioxoimidazolidin-1-ylmethyl]phenyl}-3-(4-methanesulfonylphenyl)-sulfamide

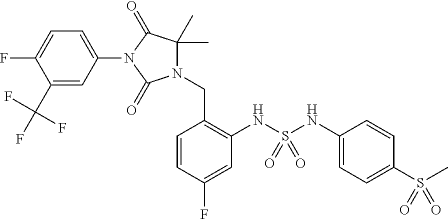

### 1) Preparation of 1-tert-butoxycarbonyl-3-{5-fluoro-2-[3-(4-fluoro-3-trifluoromethylphenyl)-5,5-dimethyl-2,4-dioxoimidazolidin-1-ylmethyl]-phenyl}sulfamide (44.2)

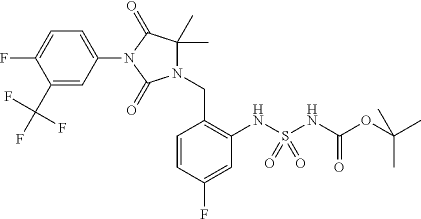 [0619] 0.35 g of compound 23.3 was dissolved at room temperature in 15 ml of dry dichloromethane, admixed with 0.28 g of N-(tert-butoxycarbonyl)sulfamoyl chloride (prepared in situ as described in G. Dewynter et al., C. R. Acad. Sci. Paris, Ser. II, 1992, 315, 1675-1682) and 0.18 ml of triethylamine and stirred at room temperature for 6 h and then left to stand overnight. For workup, the reaction mixture was concentrated under reduced pressure; the residue was purified by chromatography (method [RP2]). After freeze-drying, 1-tert-butoxycarbonyl-3-{5-fluoro-2-[3-(4-fluoro-3-trifluoromethylphenyl)-5,5-dimethyl-2,4-dioxoimidazolidin-1-ylmethyl]phenyl}sulfamide (44.2) was obtained. Molecular weight 592.14 (C₂₄H₂₅F₅N₄O₆S); retention time Rₜ=3.68 min. [E]; MS (ESI): 537.04 (MH⁺-C₄H₈).

### 2) Preparation of {5-fluoro-2-[3-(4-fluoro-3-trifluoromethylphenyl)-5,5-dimethyl-2,4-dioxoimidazolidin-1-ylmethyl]phenyl}sulfamide (44.1)

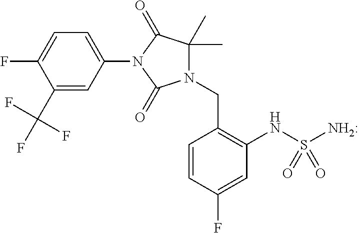 [0621] 0.41 g of compound 44.2 was dissolved at room temperature in 10 ml of dry dichloromethane, admixed with 1.06 ml of trifluoroacetic acid and 0.11 ml of water and stirred at RT for 4 h and then left to stand overnight. For workup, the reaction mixture was concentrated under reduced pressure, and the residue was dissolved in toluene and concentrated again. Finally, the residue was dissolved in dichloromethane and the organic phase was extracted by shaking with sodium hydrogencarbonate solution. The organic phase was dried over magnesium sulfate and filtered, and the filtrate was concentrated under reduced pressure. This afforded {5-fluoro-2-[3-(4-fluoro-3-trifluoromethylphenyl)-5,5-dimethyl-2,4-dioxoimidazolidin-1-ylmethyl]-phenyl}sulfamide (44.1). Molecular weight 492.08 (C₁₉H₁₇F₅N₄O₄S); retention time Rₜ=3.53 min. [D]; MS (ESI): 493.14 (MH⁺).

### 3) 1-{5-Fluoro-2-[3-(4-fluoro-3-trifluoromethylphenyl)-5,5-dimethyl-2,4-dioxoimidazolidin-1-ylmethyl]phenyl}-3-(4-methanesulfonylphenyl)sulfamide 44

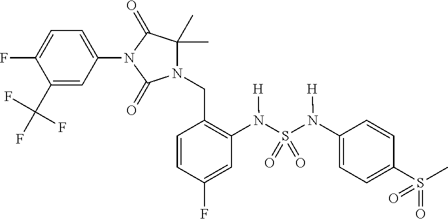 [0623] 99 mg of compound 44.1 and 0.14 g of p-bromophenyl methyl sulfone were admixed at room temperature with 12 mg of tris(dibenzylideneacetone)dipalladium(0), 10 μl of tri-(tert)-butylphosphine and 65 mg of cesium carbonate; 10 ml of dry dioxane were added under an argon atmosphere and the reaction mixture was stirred at 80° C. for 6 h. For workup, the cooled reaction mixture was filtered and the filtrate was concentrated under reduced pressure. The residue was purified by chromatography (method [RP1]) and afforded 1-{5-fluoro-2-[3-(4-fluoro-3-trifluoromethylphenyl)-5,5-dimethyl-2,4-dioxoimidazolidin-1-ylmethyl]phenyl}-3-(4-methanesulfonylphenyl)sulfamide 44. Molecular weight 646.09 (C₂₆H₂₃F₅N₄O₆S₂); retention time Rₜ=3.39 min. [E]; MS (ESI): 647.11 (MH⁺-C₄H₈).

### EXAMPLE 45

### 1-{2-[3-(4-Cyano-3-cyclopropylphenyl)-5,5-dimethyl-2,4-dioxoimidazolidin-1-ylmethyl]-5-fluorophenyl}-3-(4-ethanesulfonylphenyl)urea

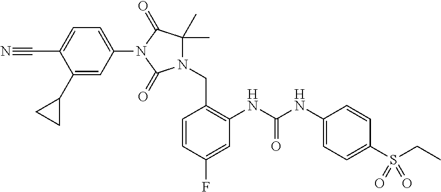 [0625] 0.32 ml of a solution of phosgene in toluene (20%) was admixed dropwise at 75° C. under an argon atmosphere with a solution of 0.12 g of compound 28.3 in 10 ml of dry acetonitrile while stirring. After the addition had ended, the mixture was stirred at 80° C. for another 2 h. For further processing, the mixture was concentrated under reduced pressure, and the residue was admixed with toluene and concentrated again under reduced pressure. The residue was dissolved in 5 ml of dry tetrahydrofuran, admixed with 83 mg of 4-ethylsulfonylaniline and 0.21 ml of triethylamine, stirred at room temperature for 4 h and then left to stand overnight. For workup, the reaction mixture was concentrated under reduced pressure and the residue was purified by chromatography (method [RP1]). This afforded 1-{2-[3-(4-cyano-3-cyclopropylphenyl)-5,5-dimethyl-2,4-dioxoimidazolidin-1-ylmethyl]-5-fluorophenyl}3-(4-ethanesulfonylphenyl)urea 45. Molecular weight 603.19 (C₃₁H₃₀FN₅O₅S); retention time Rₜ=3.36 min. [E]; MS (ESI): 604.24 (MH⁺).

In the same manner, except using methyl (4-aminophenylsulfanyl)acetate (49.1; prepared by esterification (thionyl chloride/methanol) of the corresponding acid; ¹H NMR: 7.1, d, 2H, 6.5, d, 2H, 5.3, s, 2H, 3.6, s, 3H, 3.5, s, 2H) instead of ethylsulfonylaniline and 17.3 instead of 28.3, compound 49 methyl ([4-(3-{2-[3-(4-cyano-3-trifluoromethylphenyl)-5,5-dimethyl-2,4-dioxoimidazolidin-1-ylmethyl]-5-fluorophenyl}ureido)phenylsulfanyl]acetate,

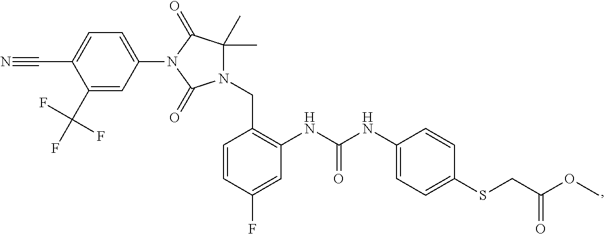

was obtained. Molecular weight 643.15 (C₃₀H₂₅F₄N₅O₅S); retention time Rₜ=3.61 min. [E]; MS (ESI): 644.10 (MH⁺).

### EXAMPLE 46

### Methyl [4-(3-{2-[3-(4-cyano-3-cyclopropylphenyl)-5,5-dimethyl-2,4-dioxoimidazolidin-1-ylmethyl]-5-fluorophenyl}ureido)benzenesulfonyl]acetate

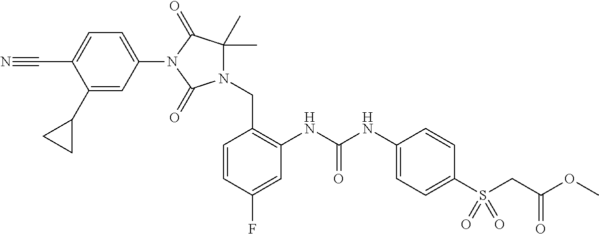 [0628] The oxidative reaction of 46.1 methyl ([4-(3-{2-[3-(4-cyano-3-cyclopropylphenyl)-5,5-dimethyl-2,4-dioxoimidazolidin-1-ylmethyl]-5-fluorophenyl} ureido)phenylsulfanyl]acetate, obtained by reaction of 28.3 with phosgene in toluene and 49.1; molecular weight 615.19 (C₃₂H₃₀FN₅O₅S); retention time Rₜ=3.58 min. [E]; MS (ESI): 616.19 (MH⁺)) with m-chloroperbenzoic acid afforded methyl [4-(3-{2-[3-(4-cyano-3-cyclopropylphenyl)-5,5-dimethyl-2,4-dioxoimidazolidin-1-ylmethyl]-5-fluorophenyl}ureido)benzenesulfonyl]acetate 46. Molecular weight 647.18 (C₃₂H₃₀FN₅O₇S); retention time Rₜ=3.36 min. [E]; MS (ESI): 648.23 (MH⁺).

### EXAMPLE 47

### [4-(3-{2-[3-(4-Cyano-3-cyclopropylphenyl)-5,5-dimethyl-2,4-dioxoimidazolidin-1-ylmethyl]-5-fluorophenyl}ureido)benzenesulfonyl]acetic acid

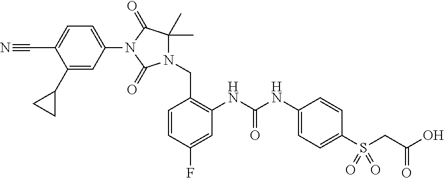 [0630] The reaction of the compound of example 46 with potassium trimethylsilanolate in tetrahydrofuran afforded the acid of example 47 ([4-(3-{2-[3-(4-cyano-3-cyclopropylphenyl)-5,5-dimethyl-2,4-dioxoimidazolidin-1-ylmethyl]-5-fluorophenyl}ureido)benzenesulfonyl]acetic acid). Molecular weight 633.16 (C₃₁H₂₈FN₅O₇S); retention time Rₜ=3.13 min. [E]; MS (ESI): 634.24 (MH⁺).

### EXAMPLE 48

### 1-{2-[3-(4-Cyano-3-trifluoromethylphenyl)-5,5-dimethyl-2,4-dioxo-imidazolidin-1-ylmethyl]-5-fluorophenyl}-3-(4-methoxycarbonyl-phenyl)sulfamide

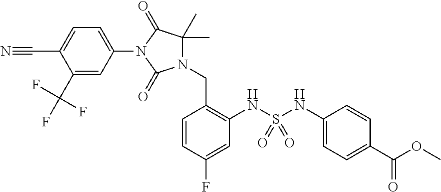

### 1) Preparation of methyl 4-sulfoaminobenzoate sodium salt (48.2)

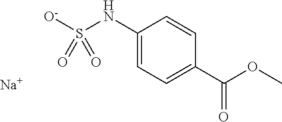 [0633] 0.45 g methyl 4-aminobenzoate was dissolved in 15 ml of dry dichloromethane and the solution was cooled to 0° C. 2.1 ml of triethylamine were added and then the mixture was admixed dropwise with 0.22 ml of chlorosulfonic acid. The reaction mixture was allowed to warm up to room temperature and stirred overnight. For workup, the mixture was concentrated under reduced pressure, and the residue was admixed with 9 ml of 1 N sodium hydroxide solution and concentrated again under reduced pressure. The crude product was taken up in hot ethanol and filtered, and the filtrate was concentrated under reduced pressure. This afforded the sodium salt of methyl 4-sulfaminobenzoate (48.2). Molecular weight 230.01 (C₈H₈NO₅), retention time Rₜ=2.01 min. [D]; MS (ES): 229.89 (M−H⁺).

### 2) Preparation of methyl 4-chlorosulfonylaminobenzoate (48.1)

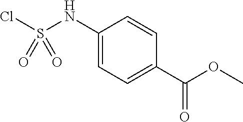 [0635] 0.1 g of the compound of example 48.2 in 5 ml of dry dichloromethane was admixed slowly with 0.17 g of phosphorus pentachloride. Thereafter, the mixture was stirred at room temperature for 4 h. For workup, the mixture was filtered and the filtrate was concentrated under reduced pressure. The residue was used in the next stage without any further purification.

### 3) 1-{2-[3-(4-Cyano-3-trifluoromethylphenyl)-5,5-dimethyl-2,4-dioxoimidazolidin-1-ylmethyl]-5-fluorophenyl}-3-(4-methoxycarbonylphenyl)sulfamide

67 mg of the compound of example 17.3 were dissolved at room temperature in 5 ml of dry dichloromethane, admixed with 80 mg of 48.1 and 45 μl of triethylamine and stirred at room temperature for 12 h. For workup, the reaction mixture was concentrated under reduced pressure and the residue was purified by chromatography (method [RP1]). This afforded 1-{2-[3-(4-cyano-3-trifluoromethylphenyl)-5,5-dimethyl-2,4-dioxoimidazolidin-1-ylmethyl]-5-fluorophenyl}-3-(4-methoxycarbonylphenyl)sulfamide 48. Molecular weight 633.13 (C₂₈H₂₃F₄N₅O₆S), retention time Rₜ=4.91 min. [D]; MS (ES): 631.94 (M−H⁺).

### EXAMPLE 50

### [4-(3-{2-[3-(4-Cyano-3-trifluoromethylphenyl)-5,5-dimethyl-2,4-dioxoimidazolidin-1-ylmethyl]-5-fluorophenyl}ureido)phenylsulfanyl]acetic acid

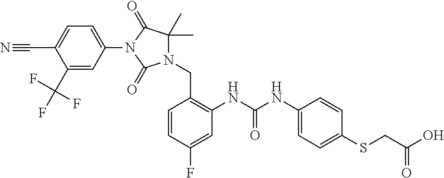 [0638] Analogously to the procedure for the preparation of the compound of example 18, the ester 49 was reacted with potassium trimethylsilanolate to obtain 50. Molecular weight 629.13 (C₂₉H₂₃F₄N₅O₅S); retention time Rₜ=3.69 min. [D]; MS (ESI): 630.14 (MH

### EXAMPLE 54

### Methyl 4-(3-{2-[3-(2-chloropyridin-4-yl)-5,5-dimethyl-2,4-dioxo-imidazolidin-1-ylmethyl]-5-fluorophenyl}ureido)benzoate

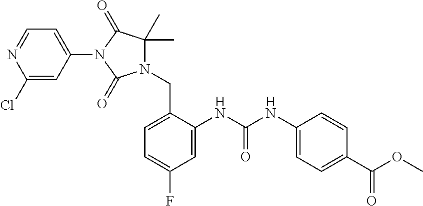

### 1) Preparation of 3-(2-chloropyridin-4-yl)-5,5-dimethylimidazolidine-2,4-dione (54.3)

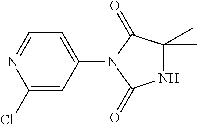 [0641] 1.35 g of 2-chloro-4-aminopyridine were dissolved at room temperature in 10 ml of dry pyridine, admixed with 1.65 g of methyl 2-isocyanato-2-methylpropionate, stirred at 80° C. for 20 h over several days and each time left to stand at room temperature overnight. The reaction mixture was concentrated under reduced pressure and the residue was purified by chromatography (method [RP1]). This afforded 3-(2-chloropyridin-4-yl)-5,5-dimethylimidazolidine-2,4-dione (54.3). Molecular weight 239.04 (C₁₀H₁₀N₃O₂), retention time Rₜ=2.75 min. [E]; MS (ESI): 240.06 (MH

### 2) Preparation of 3-(2-chloropyridin-4-yl)-1-(4-fluoro-2-nitrobenzyl)-5,5-dimethylimidazolidine-2,4-dione (54.2)

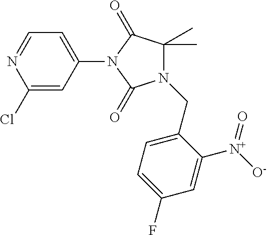 [0643] Compound 54.2 was obtained analogously to the preparation of compound 23.11 by reaction of 54.3 with 23.10. Molecular weight 392.06 (C₁₇H₁₄ClFN₄O₄), retention time Rₜ=3.33 min. [E]; MS (ESI): 393.10 (MH⁺).

### 3) Preparation of 1-(2-amino-4-fluorobenzyl)-3-(2-chloropyridin-4-yl)-5,5-dimethylimidazolidine-2,4-dione (54.1)

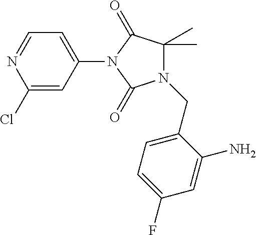 [0645] Compound 54.1 was obtained analogously to the procedure in the preparation of compound 23.3 by reaction of 54.2 with palladium hydroxide on carbon and trimethylamine-borane. Molecular weight 362.09 (C₁₇H₁₆ClFN₄O₂), retention time Rₜ=3.15 min. [E]; MS (ESI): 363.08 (MH⁺).

### 4) Preparation of methyl 4-(3-{2-[3-(2-chloropyridin-4-yl)-5,5-dimethyl-2,4-dioxoimidazolidin-1-ylmethyl]-5-fluorophenyl}ureido)benzoate (54)

As described for the preparation of compound 1.4, the compound of example 54 was obtained by reaction of 54.1 with methyl 4-isocyanatobenzoate. Molecular weight 539.13 (C₂₆H₂₃ClFN₅O₅), retention time Rₜ=3.41 min. [E]; MS (ESI): 540.17 (MH⁺).

The compound of example 59,

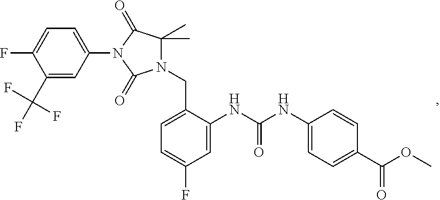

methyl 4-(3-{5-fluoro-2-[3-(4-fluoro-3-trifluoromethylphenyl)-5,5-dimethyl-2,4-dioxoimidazolidin-1-ylmethyl]phenyl}ureido)benzoate (molecular weight 590.15 (C₂₈H₂₃F₅N₄O₅); retention time Rₜ=3.68 min. [E]; MS (ESI): 591.16 (MH⁺)), was obtained like compound 1.4 by reaction of compound 23.3 with methyl 4-isocyanatobenzoate.

The compounds of examples 57,

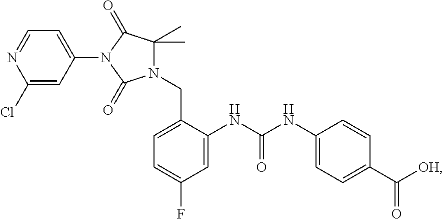

4-(3-{2-[3-(2-chloropyridin-4-yl)-5,5-dimethyl-2,4-dioxoimidazolidin-1-ylmethyl]-5-fluorophenyl}ureido)benzoic acid (molecular weight 525.12 (C₂₅H₂₁F₅N₅O₅); retention time Rₜ=3.03 min. [E]; MS (ESI): 526.09 (MH⁺)), and 60,

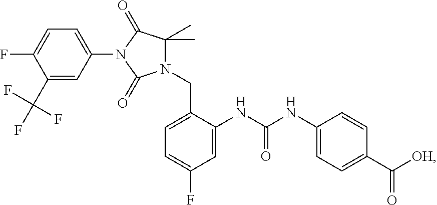

4-(3-{5-fluoro-2-[3-(4-fluoro-3-trifluoromethylphenyl)-5,5-dimethyl-2,4-dioxoimidazolidin-1-ylmethyl]phenyl}-ureido)benzoic acid (molecular weight 576.14 (C₂₇H₂₁F₅N₄O₅); retention time Rₜ=3,33 min. [E]; MS (ESI): 577.11 (MH')), were, as described for example 18, obtained from the methyl esters 54 and 59 by reaction with potassium trimethylsilanolate.

### EXAMPLE 61

### N-[4-(3-{5-Fluoro-2-[3-(4-fluoro-3-trifluoromethylphenyl)-5,5-dimethyl-2,4-dioxoimidazolidin-1-ylmethyl]phenyl}ureido)benzoyl]methanesulfonamide

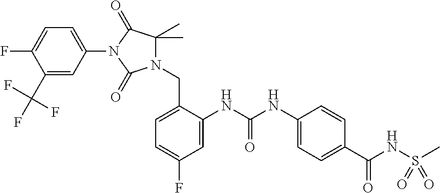 [0650] To prepare the compound of example 61, 0.17 g of compound 60 was dissolved at room temperature in 10 ml of dry dichloromethane and admixed while stirring with 0.14 g of N-(3-dimethylaminopropyl)-N′-ethylcarbodiimide (EDC) and 0.11 g of dimethylaminopyridine (DMAP). To this mixture were added 29 mg of methanesulfonamide, and the mixture was then stirred at room temperature for 4 h. The reaction mixture was concentrated under reduced pressure; the residue was taken up in a little water and acidified with 1 N hydrochloric acid. The precipitated product was filtered off with suction, washed with water and dried. Chromatographic purification (method [RP1]) afforded N-[4-(3-{5-fluoro-2-[3-(4-fluoro-3-trifluoromethylphenyl)-5,5-dimethyl-2,4-dioxoimidazolidin-1-ylmethyl]-phenyl}ureido)benzoyl]methanesulfonamide 61. Molecular weight 653.13 (C₂₈H₂₄F₅N₅O₆S); retention time Rₜ=3.33 min. [E]; MS (ESI): 654.11 (MH⁺).

The compounds of example 62,

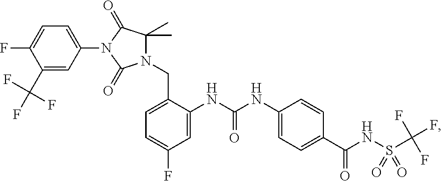

C,C,C-trifluoro-N-[4-(3-{5-fluoro-2-[3-(4-fluoro-3-trifluoromethylphenyl)-5,5-dimethyl-2,4-dioxoimidazolidin-1-ylmethyl]phenyl}ureido)benzoyl]methanesulfonamide, (molecular weight 707.10 (C₂₈H₂₁F₈N₅O₆S); retention time Rₜ=3.33 min. [E]; MS (ESI): 708.15 (MH⁺)); of example 63,

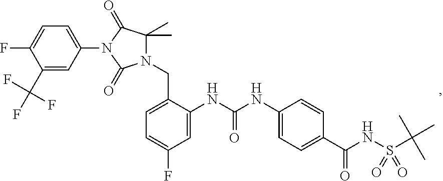

N-[4-(3-{5-fluoro-2-[3-(4-fluoro-3-trifluoromethylphenyl)-5,5-dimethyl-2,4-dioxoimidazolidin-1-ylmethyl]-phenyl}ureido)benzoyl]-2-methylpropane-2-sulfonamide, (molecular weight 695.18 (C₃₁H₃₀F₅N₅O₆S); retention time Rₜ=3.48 min. [E]; MS (ESI): 696.18 (MH⁺)); of example 64,

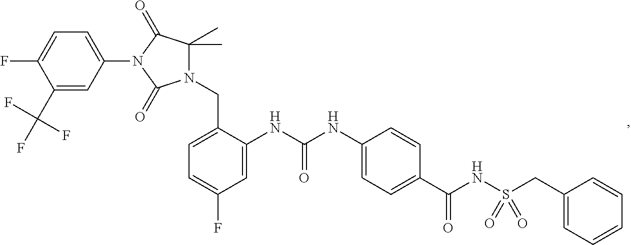

N-[4-(3-{5-fluoro-2-[3-(4-fluoro-3-trifluoromethylphenyl)-5,5-dimethyl-2,4-dioxoimidazolidin-1-ylmethyl]phenyl}ureido)benzoyl]-C-phenylmethanesulfonamide, (molecular weight 729.16 (C₃₄H₂₈F₅N₅O₆S); retention time Rₜ=3.58 min. [E]; MS (ESI): 730.19 (MH⁺)); and of example 65,

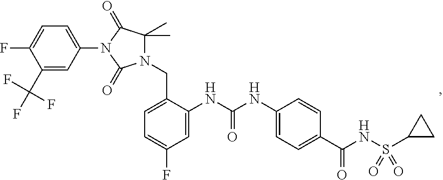

N-[4-(3-{5-fluoro-2-[3-(4-fluoro-3-trifluoromethylphenyl)-5,5-dimethyl-2,4-dioxoimidazolidin-1-ylmethyl]phenyl}ureido)benzoyl]cyclopropanesulfonamide, (molecular weight 679.15 (C₃₀H₂₆F₅N₅O₆S); retention time Rₜ=3.41 min. [E]; MS (ESI): 680.17 (MH⁺));
were prepared analogously to the procedure for compound 61: trifluoromethanesulfonamide was used for 62, tert-butylsulfonamide for 63, benzylsulfonamide for 64 and cyclopropanesulfonamide for 65.

### EXAMPLE 66

### 1-{2-[3-(4-Cyano-3-cyclopropylphenyl)-5,5-dimethyl-2,4-dioxoimidazolidin-1-ylmethyl]-4-trifluoromethylphenyl}-3-(4-methanesulfonylphenyl)urea

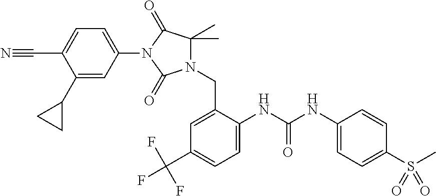

### 1) Preparation of 4-[3-(2-bromo-5-trifluoromethylbenzyl)-4,4-dimethyl-2,5-dioxoimidazolidin-1-yl]-2-cyclopropylbenzonitrile (66.3)

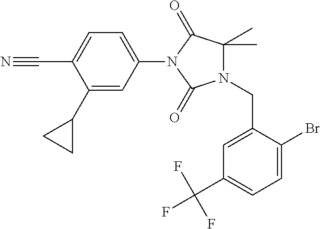 [0654] Under conditions as described for the preparation of 1.2, compound 28.1 was reacted with 2-bromo-1-bromomethyl-5-trifluoromethylbenzene and afforded 66.3. Molecular weight 505.06 (C₂₃H₁₉BrF₃N₃O₂); retention time Rₜ=3.85 min. [E]; MS (ESI): 506.09 (MH⁺).

### 2) Preparation of 4-[3-(2-amino-5-trifluoromethylbenzyl)-4,4-dimethyl-2,5-dioxoimidazolidin-1-yl]-2-cyclopropylbenzonitrile trifluoroacetic acid salt (66.2)

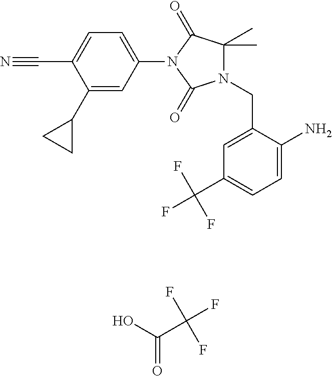 [0656] Analogously to the procedure as described for the preparation of 1.3, 66.3 was reacted with benzophenone imine to give 66.2. The compound was isolated as the salt with trifluoroacetic acid. Molecular weight (free base) 442.16 (C₂₃H₂₁F₃N₄O₂); retention time Rₜ=3.13 min. [E]; MS (ESI): 443.17 (MH⁺).

### 3) Preparation of 1-{2-[3-(4-cyano-3-cyclopropylphenyl)-5,5-dimethyl-2,4-dioxoimidazolidin-1-ylmethyl]-4-trifluoromethylphenyl}-3-(4-methylsulfanyl-phenyl)urea (66.1)

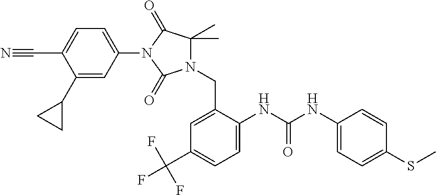 [0658] Compound 66.1 was obtained as described for compound 35 by reaction of compound 66.2 with 1-isocyanato-4-methylsulfanylbenzene. Molecular weight 607.28 (C₃₁H₂₈F₃N₅O₃S); retention time Rₜ=2.75 min. [E]; MS (ESI): 608.23 (MH

### 4) Preparation of 1-{2-[3-(4-cyano-3-cyclopropylphenyl)-5,5-dimethyl-2,4-dioxoimidazolidin-1-ylmethyl]-4-trifluoromethylphenyl}-3-(4-methane-sulfonylphenyl)urea

Analogously to the procedure for the preparation of the compound of example 13, for the preparation of the compound of example 66, compound 66.1 was oxidized with m-chloroperbenzoic acid. Molecular weight 639.17 (C₃₁H₂₈F₃N₅O₅S); retention time Rₜ=2.49 min. [E]; MS (ESI): 640.19 (MH⁺).

In an analogous manner, the compound of example 67

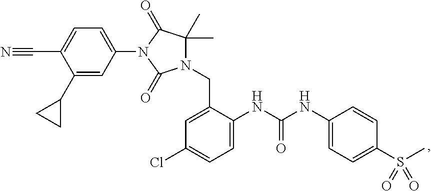

1-{4-chloro-2-[3-(4-cyano-3-cyclopropylphenyl)-5,5-dimethyl-2,4-dioxoimidazolidin-1-ylmethyl]phenyl}-3-(4-methanesulfonylphenyl)urea (molecular weight 605.15 (C₃₀H₂₈ClN₅O₅S); retention time Rₜ=2.40 min. [E]; MS (ESI): 606.17 (MH⁺)) was obtained via the reaction sequence of 5-chloro-2-nitrobenzyl alcohol→1-bromomethyl-5-chloro-2-nitrobenzene (bromination with phosphorus tribromide in dichloromethane; ¹H NMR: 8.12, d, 1H, 7.9, s, 1H, 7.72, d, 1H, 4.9, s, 2H)→4-[3-(5-chloro-2-nitrobenzyl)-4,4-dimethyl-2,5-dioxoimidazolidin-1-yl]-2-cyclopropylbenzonitrile (67.3; molecular weight 438.10 (C₂₂H₁₉ClN₄O₄); retention time Rₜ=3.10 min. [E]; MS (ESI): 439.16 (MH⁺))→4-[3-(2-amino-5-chlorobenzyl)-4,4-dimethyl-2,5-dioxoimidazolidin-1-yl]-2-cyclopropylbenzonitrile salt with trifluoroacetic acid (67.2; free base: molecular weight 408.13 (C₂₂H₂₁ClN₄O₂); retention time Rₜ=2.47 min. [E]; MS (ESI): 409.14 (MH⁺))→1-{4-chloro-2-[3-(4-cyano-3-cyclopropylphenyl)-5,5-dimethyl-2,4-dioxoimidazolidin-1-ylmethyl]phenyl}-3-(4-methylsulfanylphenyl)urea (67.1; molecular weight 573.16 (C₃₁H₂₈ClN₅O₃S); retention time Rₜ=4.85 min. [D]; MS (ESI): 574.37 (MH⁺))→1-{4-chloro-2-[3-(4-cyano-3-cyclopropylphenyl)-5,5-dimethyl-2,4-dioxoimidazolidin-1-ylmethyl]phenyl}-3-(4-methanesulfonylphenyl)urea (67).

The compound of example 68,

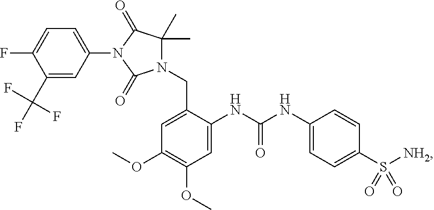

4-(3-{2-[3-(4-fluoro-3-trifluoromethylphenyl)-5,5-dimethyl-2,4-dioxoimidazolidin-1-ylmethyl]-4,5-dimethoxyphenyl}ureido)benzenesulfonamide (molecular weight 653.17 (C₂₈H₂₇F₄N₅O₇S); retention time Rₜ=2.51 min. [E]; MS (ESI): 654.17 (MH⁺)) was obtained via the reaction sequence of 3-(4-fluoro-3-trifluoromethylphenyl)-5,5-dimethylimidazolidine-2,4-dione (23.1)→1-(4,5-dimethoxy-2-nitrobenzyl)-3-(4-fluoro-3-trifluoromethylphenyl)-5,5-dimethylimidazolidine-2,4-dione (68.3, alkylation with 4,5-dimethoxy-2-nitrobenzyl bromide with cesium carbonate in acetonitrile; molecular weight 485.12 (C₂₁H₁₉F₄N₃O₆); retention time Rₜ=4.73 min. [D]; MS (ESI): 486.09 (MH⁺))→1-(2-amino-4,5-dimethoxybenzyl)-3-(4-fluoro-3-trifluoromethylphenyl)-5,5-dimethylimidazolidine-2,4-dione (68.2, reduction of 68.3 with palladium hydroxide on carbon and trimethylamineborane in methanol; molecular weight 455.14 (C₂₁H₂₁F₄N₃O₄); retention time Rₜ=2.12 min. [E]; MS (ESI): 456.12 (MH⁺))→4-(3-{2-[3-(4-fluoro-3-trifluoromethylphenyl)-5,5-dimethyl-2,4-dioxoimidazolidin-1-ylmethyl]-4,5-dimethoxyphenyl}ureido)benzenesulfonyl chloride (68.1, obtained analogously to the procedure as described in the preparation of 1.4 by reaction with 4-isocyanatobenzenesulfonyl chloride; the compound was used in the next stage without any further purification)→4-(3-{2-[3-(4-fluoro-3-trifluoromethylphenyl)-5,5-dimethyl-2,4-dioxoimidazolidin-1-ylmethyl]-4,5-dimethoxyphenyl}ureido)benzenesulfonamide, 68 (obtained from the sulfonyl chloride 68.1 by reaction with ammonia in methanol analogously to the procedure as described in 1.5).

In a similar manner, the compound of example 69,

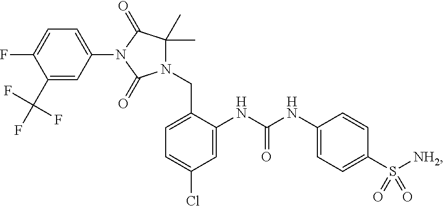

4-(3-{5-chloro-2-[3-(4-fluoro-3-trifluoromethylphenyl)-5,5-dimethyl-2,4-dioxoimidazolidin-1-ylmethyl]phenyl}ureido)benzenesulfonamide (molecular weight 627.09 (C₂₆H₂₂ClF₄N₅O₅S); retention time Rₜ=2.52 min. [E]; MS (ESI): 628.11 (MH⁺)) was prepared: 3-(4-fluoro-3-trifluoromethylphenyl)-5,5-dimethylimidazolidine-2,4-dione (23.1)→1-(2-bromo-4-chlorobenzyl)-3-(4-fluoro-3-trifluoromethylphenyl)-5,5-dimethylimidazolidine-2,4-dione (69.3, by reaction of 23.1 with 2-bromo-4-chlorobenzyl bromide; ¹H NMR: 8.0, m, 1H, 7.9, m, 1H, 7.8, s, 1H, 7.7, t, 1H, 7.6, d, 1H, 7.48, d, 1H, 4.6, s, 2H, 1.4, s, 6H)→1-(2-amino-4-chlorobenzyl)-3-(4-fluoro-3-trifluoromethylphenyl)-5,5-dimethylimidazolidine-2,4-dione (69.2, by reaction with benzophenone imine analogously to the procedure in the preparation of 1.3; molecular weight 429.08 (C₁₉H₁₆ClF₄N₃O₂); retention time Rₜ=2.65 min. [E]; MS (ESI): 430.08 (MH⁺))→4-(3-{5-chloro-2-[3-(4-fluoro-3-trifluoromethylphenyl)-5,5-dimethyl-2,4-dioxoimidazolidin-1-ylmethyl]phenyl}ureido)benzenesulfonyl chloride (69.1, by reaction with 4-isocyanatobenzenesulfonyl chloride; the compound was used in the next stage without any further purification)→4-(3-{5-chloro-2-[3-(4-fluoro-3-trifluoromethylphenyl)-5,5-dimethyl-2,4-dioxoimidazolidin-1-ylmethyl]phenyl}-ureido)benz enesulfonamide 69 (obtained from the sulfonyl chloride 69.1 by reaction with ammonia in methanol analogously to the procedure as described in 1.5).

The compound of example 70,

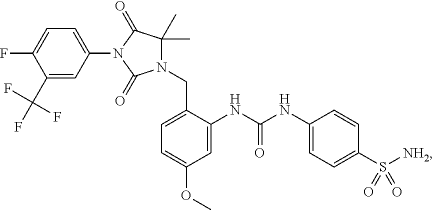

4-(3-{2-[3-(4-fluoro-3-trifluoromethylphenyl)-5,5-dimethyl-2,4-dioxoimidazolidin-1-ylmethyl]-5-methoxyphenyl}ureido)benzenesulfonamide (molecular weight 623.14 (C₂₇H₂₅F₄N₅O₆S); retention time Rₜ=2.31 min. [α]; MS (ESI): 624,12 (MH⁺)) was also obtained via a similar reaction sequence: 3-(4-fluoro-3-trifluoromethylphenyl)-5,5-dimethylimidazolidine-2,4-dione (23.1)→3-(4-fluoro-3-trifluoromethylphenyl)-1-(4-methoxy-2-nitrobenzyl)-5,5-dimethylimidazolidine-2,4-dione (70.3, by reaction of 23.1 with 1-bromomethyl-4-methoxy-2-nitrobenzene; molecular weight 455.11 (C₂₀H₁₇F₄N₃O₅); retention time Rₜ=3.95 min. [D]; MS (ESI): 456,03 (MH⁺))→1-(2-amino-4-methoxybenzyl)-3-(4-fluoro-3-trifluoromethylphenyl)-5,5-dimethylimidazolidine-2,4-dione (70.2, reduction of 70.3 with palladium hydroxide on carbon and trimethylamine-borane in methanol; molecular weight 425.13 (C₂₀H₁₉F₄N₃O₃); retention time Rₜ=2.34 min. [C]; MS (ESI): 426.06 (MH⁺))→4-(3-{2-[3-(4-fluoro-3-trifluoromethylphenyl)-5,5-dimethyl-2,4-dioxoimidazolidin-1-ylmethyl]-5-methoxyphenyl}ureido)benzenesulfonyl chloride (70.1, by reaction with 4-isocyanatobenzenesulfonyl chloride; the compound was used in the next stage without any further purification)→4-(3-{2-[3-(4-fluoro-3-trifluoromethylphenyl)-5,5-dimethyl-2,4-dioxoimidazolidin-1-ylmethyl]-5-methoxyphenyl}ureido)benzenesulfonamide 70 (obtained from the sulfonyl chloride 70.1 by reaction with ammonia in methanol analogously to the procedure as described in 1.5).

### Pharmacological Testing:

### In Vitro Tests:

In vitro functional assays with recombinant cells:

Function-testing assays were performed by means of the FLIPR technique (“Fluorometric Imaging Plate Reader”, Molecular Devices Corp.).

To this end, ligand-induced changes in the intracellular concentration of Ca²⁺ in recombinant HEK293 cells, which expressed both a cannabinoid receptor (CB1 or CB2) and G-protein Galphal6, were determined. For the studies, cells were sown into 96-well microtiter plates (60 000 cells/well) and left to grow overnight. The medium was removed and the cells were incubated in buffer which contained the fluorescent dye Fluo-4. After this loading with dye, the cells were washed, test substance was added dissolved in buffer, the mixture was incubated for 20 minutes, a known cannabinoid receptor agonist as a reference agonist was added in buffer and, finally, the changes in the intracellular Ca²⁺ concentration were measured in the FLIPR unit.

Results were presented as the percentage change relative to the control (0%: analogous experiment without test substance and without reference agonist, i.e. only with buffer; 100%: analogous experiment without test substance, but with reference agonist in excess), and used to calculate dose/action curves, and IC₅₀ values were determined.

### Results:

The values of the functional assay compared to the cannabinoid 1 receptor including illustrative selectivities compared to the cannabinoid 2 receptor can be taken from table 1 which follows.

TABLE 1

| Example No. | hCB1R: FLIPR; IC₅₀ [nM] | hCB2R: FLIPR; IC₅₀ [nM] |
|---|---|---|
| 1 | 24 | >10000 |
| 2 | 220 | >10000 |
| 3 | 19 | >10000 |
| 4 | 109 | >10000 |
| 6 | 11 | >10000 |
| 8 | 35 | >10000 |
| 10 | 53 | >10000 |
| 16 | 12 | |
| 17 | 10 | |
| 19 | 9 | |
| 20 | 16 | |
| 22 | 9 | |
| 25 | 58 | |
| 26 | 66 | |
| 27 | 13 | |
| 28 | 9 | |
| 29 | 69 | |
| 30 | 11 | |
| 30.1 | 12 | |
| 31 | 32 | |
| 41 | 4 | |
| 43 | 27 | |
| 47 | 25 | |
| 49 | 2 | |
| 50 | 13 | |
| 60 | 53 | |
| 61 | 16 | |
| 62 | 46 | |
| 64 | 39 | |
| 65 | 42 | |
| 66 | 10 | |
| 67 | 6 | |
| 68 | 39 | |
| 69 | 11 | |
| 70 | 14 | |

### Binding to the CB 1 Receptor:

Test compounds: The compounds (3 μl, 10 mM, 100% DMSO), pipetted into 96-well PP microtiter plates, were diluted with 27 μl of 100% DMSO (dimethyl sulfoxide). Proceeding from this solution, further 3-fold dilution steps were undertaken by transferring 10 μl in each case to a new PP microtiter plate and adding a further 20 μl of 100% DMSO. In each case 6 μA of these solutions were transferred into new 96-well PP microtiter plates and made up with 144 μl of assay buffer. The end concentrations ranged from 10 μM to 0.005 μM.
Negative control: AM 251, dissolved in assay buffer with 1% DMSO, was added to the dilution series in the microtiter plates as a control. The end concentration was 1 μM.
Blank control: assay buffer with 1% DMSO was added to the dilution series of the microtiter plates as a blank control.

### Summary of the Assay Parameters:

| | | |
|---|---|---|
| Assay volume | | 200 μl |
| Receptor | CHO-K1/cannabinoid CB1 | 2 μg/well |
| | Protein | |
| Ligand | [³H]-SR141716A | 0.5 nM |
| | | 0.0195 μCi/well |
| Ions | Tris-HCl | 50 mM, pH 7.4 |
| | MgCl₂ | 5 mM |
| | EDTA | 2.5 mM |
| | BSA (fatty acid-free) | 0.2% |
| Nonspecific binding | AM 251 | 1 μM |
| Compound | in 1% DMSO | 10 μM to 0.0050 μM |

Analysis of the data:
High control: ³H binding without addition of the compound
Low control: ³H binding in the presence of 1 μM AM 251

The values were calculated using the corrected raw data.

$$Inhibition of ligand binding ( \% ) = 100 * ( 1 - \frac{( sample - lowcontrol}{( highcontrol - lowcontrol )} )$$

The values reported were obtained as average values of a double determination. The IC₅₀ values were calculated from the measurements with the program Xlfit, formula 205. Ki values were obtained from the IC₅₀ and Kd values utilizing the Cheng-Prusoff equation:

$$Ki = \frac{IC 50}{1 + \frac{C}{Kd}} ( C = concentration of the radioligand )$$

Literature: Cheng, Y.-C., and Prusoff, W. H. (1973) Biochem. Pharmacol 22, 3099-3108 Results: Kᵢ values of example compounds; Table 2:

| Example No. | hCB1R; Bindung Kᵢ [nM] |
|---|---|
| 1 | 20 |
| 3 | 11 |
| 7 | 73 |
| 9 | 32 |
| 12 | 11 |
| 13 | 7 |
| 15 | 58 |
| 18 | 68 |
| 21 | 34 |
| 23 | 17 |
| 24 | 9 |
| 32 | 40 |
| 42 | 35 |
| 43 | 17 |
| 45 | 6 |
| 46 | 7 |
| 49 | 11 |
| 59 | 29 |

It can be seen from the test data that the inventive compounds of the formula I act as CB1R antagonists and are therefore very suitable for treating metabolic syndrome, type II diabetes and obesity.

### In Vivo Tests:

### “Milk Consumption in Mice”

The test is used to study the anorexigenic potency of the test substances. Female NMRI mice, 25-35 g in weight, are used. The mice are accustomed to the housing conditions for at least one week and to the condensed milk supplied for 2 days.

The feed is removed from the mice for 24 hours, but they have constant access to water. On the day of the experiment, the animals are put in individual cages; the cage lids can accommodate the pipettes filled with milk. The test substances are administered orally, intraperitoneally or subcutaneously. After the administration, the mice are put in their cages and receive access to the milk 30 min later. The milk consumption is read off every 30 min over 7 hours; at the same time, obvious changes in behavior of the animals are noted.

“Antagonization of CB 1-mediated Hypothermia”

The test is used to measure the potency of cannabinoid CB1 receptor (CB1) antagonists. What is measured is the extent to which the CB1 antagonists to be tested are capable of preventing or of antagonizing hypothermia induced by a CB1 agonist.

Female NMRI mice, 25-35 g in weight, are used. The mice are accustomed to the housing conditions for at least one week.

At time 0 min, the animals are treated orally, intravenously or intraperitoneally with the CB1 antagonist to be tested. 30 min later, the CB1 agonist CP55.940, 1.25 mg/kg, is administered to the mice intraperitoneally. This brings about a fall in the body temperature by 5-6° C. within 30 min. The body temperature is measured rectally for the first time 30 min before the test substance administration and then every 30 min after this administration, if appropriate immediately before a substance administration, over 4 hours.

The potency of the test substances is reported as the percentage decrease in the area under the temperature-time curve which is formed firstly by the average basal temperature, and secondly by the temperature-time curve, of the animals treated exclusively with the CB1 antagonist.

### “Intestinal Motility in Mice”

The method serves firstly to study the influence of test substances themselves on the small intestinal motility, and secondly to study to what extent specifically induced effects on the small intestinal motility can be prevented or antagonized, for example the delay in the intestinal passage by the cannabinoid CB1 agonist CP55.940.

Female NMRI mice with a weight of 25-35 g are used. The mice are accustomed to the housing conditions for at least one week.

The feed is removed from the mice for 24 hours, but they have constant access to water. The test substances are administered orally, intravenously, subcutaneously, but not intraperitoneally. If a specific effect is to be antagonized, the test substance is administered 30-120 min before the specific effector. 30 min after this administration, a defined amount of a dyed, non-caloric filler is introduced into the stomach by gavage. After a further 30 min (the dyed filler has about 80% filled the small intestine at this point), the animals are sacrificed and the small intestine is dissected. The intestinal motility is reported as the passage of the dyed filler compared to the total length of the small intestine in percent. A treatment effect is reported as the difference of this passage to the vehicle control, likewise in percent.

1. A compound of the formula I 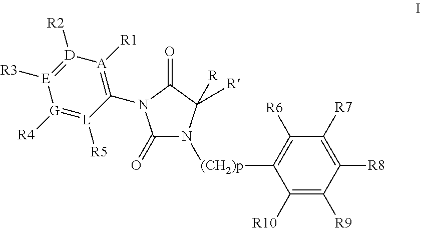
in which
R, R′ are each independently H, (CH₂)ₙ-aryl, (C₁-C₆)-alkyl, where (C₁-C₆)-alkyl or the aryl radical may be substituted by halogen, O—R14, S(O)ₘ—R12 or NR13R15;
or R and R′ together form a ring having from three to eight carbon atoms, where one carbon atom may be replaced by O, S(O)ₘ, NR13 or NR15;
m is 0, 1, 2;
n is 0, 1, 2, 3, 4;
p is 1, 2, 3, 4, 5;
q is 1, 2, 3, 4;
r is 2, 3, 4, 5, 6;
v is 0, 1, 2, 3, 4;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, N₃, NC, NO₂, CF₃, (C₁-C₈)-alkyl, (C₃-C₈)-cycloalkyl, (CH₂)_{q}-[(C₃-C₈)-cycloalkyl], (CH₂)ₙ-[(C₃-C₈)-cycloalkenyl], (CH₂)ₙ-[(C₇-C₁₂)-bicycloalkyl], (CH₂)ₙ-[(C₇-C₁₂)-tricycloalkyl], adamantan-1-yl, adamantan-2-yl, (CH₂)ₙ-aryl, (CH₂)ₙ-heteroaryl, OCF₃, O—R11, NR13R15, NH—CN, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₈)-cycloalkyl], CO—O—(CH₂)ₙ-aryl, CO—O—(CH₂)ₙ-heteroaryl, CO—NH₂, CO—NH—CN, CO—NH—[(C₁-C₈)-alkyl], CO—N[(C₁-C₈)-alkyl]₂, CO—NH-[(C₃-C₈)-cycloalkyl], CO—N[(C₃-C₈)-cycloalkyl]₂, C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₈)-alkyl, CO—(C₃-C₈)-cycloalkyl, CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O—R12, CH₂—O—(CH₂)ₙ—CO—O[(C₁-C₈)-alkyl], CH₂—O—(CH₂)ₙ—CO—NH₂, CH₂—O—(CH₂)_{q}—COOH, where the alkyl, cycloalkyl, cycloalkenyl, bicycloalkyl and tricycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, OCF₃, OH, O—(CH₂)ₙ-aryl, (CH₂)ₙ-aryl, S(O)ₘ—(C₁-C₆)-alkyl, SO₂—NH₂, SH, NR12R13, NH—CO—[(C₁-C₆)-alkyl], NH—CO—(CH₂)ₙ-aryl, (CH₂)ₙ—COOH, (CH₂)ₙ—CONH₂, (CH₂)ₙ—CO—O(C₁-C₆)-alkyl, (CH₂)ₙ—CO—(C₁-C₆)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ᵣ—OH, O—R13, OCF₃, (CH₂)ₙ—O—(CH₂)ᵣ—NH₂, (CH₂)ₙ—NH—R11, (CH₂)ₙ—NRCH₂)_{q}—CO—O(C₁-C₆)-alkylk, (CH₂)ₙ—NRCH₂)_{q}—COOK, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12-CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N [(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂, 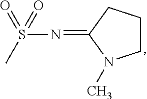
SO₂—NH—CO—R12, SO₂—NHR12, SO₂—NH—(CH₂)ᵣ—OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)—NH₂, (CH₂)ₙ—C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms, and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙO—(CH₂)ₙ—CR21R22-CONH₂, (CH₂)ₙ—O—(CH₂)ₙ—CR21R22-CONH₂, (CH₂)ₙ—O—(CH₂)ₙ—CO—R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂), —SO₂—R16, SO₂—N═CH—N(CH₃)₂, 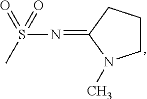
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CH, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as T-bicyclic heterocycle, T-aryl or T-heteroaryl and where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23-C(═NR13)-NR24, NR23-C(═NR22)-NR24, CO—NR23-CR22R23, CO—CR22R23-CO, CR22R23-CO—CR22R24, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CO—NR23-SO₂, SO₂—NR23-CO, CO—CR22R23-SO₂, SO₂—CR22R23-CO, CR23R24-CR23R24-CR23R24, CR23R24-NR23-CR23R24;
R11 is H, (C₁-C₈)-alkyl, (C₂-C₁₀)-alkenyl, (C₂-C₁₀)-alkynyl, (C₃-C₈)-cycloalkyl, (CH₂)_{q} [(C₃-C₈)-cycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O—[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21(CONH₂)₂, (CH₂)ₙ—CR21(COOH)₂, (CH₂)ₙ—CR21R22-CO—O—[(C₁-C₆)-alkyl], (CH₂)ₙ—CR21R22—CONH₂, (CH₂)ₙ—CR21R22-CO—NH—[(C₁-C₈)-alkyl], (CH₂)ₙ—CR21R22-CO—N[C₁-C₈)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R12 is H, (C₁-C₈)-alkyl, (C₃-C₈)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl,
where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ—[(C₁-C₆)-alkyl], SO₂—NH₂, COOH, CONH₂, CO—[O(C₁-C₆)-alkyl], CO—(C₁-C₆)-alkyl and where the alkyl radicals may be substituted by fluorine atoms;
R14 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], (CH₂)ₙ—CO—[O—(CH₂)ₙ-aryl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—COOH, where the alkyl and cycloalkyl radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R15 is H, (C₁-C₈)-alkyl, (C₃-C₈)-cycloalkyl, (CH₂)ₙ-aryl, (CH₂)ₙ-heteroaryl, (CH₂)ₙ—CO—[O—(C₁-C₈)-alkyl], CO—[(C₁-C₈)-alkyl], CO—[(C₃-C₈)-cycloalkyl], CO-aryl, CO-heteroaryl, (CH₂)ₙ—CO—NH₂, (CH₂)_{q}—COOH, (CH₂)ₙ—SO₂—NH₂, (CH₂)ₙ—C(CH₃)₂—COOH, where the alkyl and cycloalkyl radicals may be substituted by fluorine atoms and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholin-4-yl, thiomorpholine-1,1-dioxid-4-yl, NH—(CH₂), —OH, NH—CH(CH₂OH)₂, NH—C(CH₂OH)₃, N[(C₁-C₆)-alkyl-OH]₂, N[(C₁-C₆)-alkyl][(C₁-C₆)-alkyl-OH], D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-CO—O(C₁-C₆)-alkyl, N[(C₁-C₆)-alkyl][(C₁-C₈)-alkyl]-COOH, N[(C₁-C₆)-alkyl][(C₁-C₈)-alkyl]-CONH₂, NH—[C(H)(aryl)]-CO—O(C₁-C₆)-alkyl, NH—[C(H)(aryl)]-COOH, NH—[C(H)(aryl)]-CONH₂, N[(C₁-C₆)-alkyl][C(H)(aryl)]-CO—O(C₁-C₆)-alkyl, N[(C₁-C₆)-alkyl][C(H)(aryl)]-COOH, N[(C₁-C₆)-alkyl][C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, N[(C₁-C₆)-alkyl][C(H)(heteroaryl)]-CO—O(C₁-C₆)-alkyl, N[(C₁-C₆)-alkyl][C(H)(heteroaryl)]-COOH, N[(C₁-C₆)-alkyl][C(H)(heteroaryl)]-CONH₂, N[(C₁-C₆)-alkyl][(C₃-C₈)-cycloalkyl]-CO—O(C₁-C₆)-alkyl, N[(C₁-C₆)-alkyl][(C₃-C₈)-cycloalkyl]-COOH, N[(C₁-C₆)-alkyl][(C₃-C₈)-cycloalkyl]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(C₁-C₈)-alkyl-OH, NH—[(C₁-C₆)-alkyl]-SO₂—(C₁-C₆)-alkyl, NH—[(C₁-C₆)-alkyl]-SO₃H, NH—[(C₁-C₆)-alkyl]-SO₂—NH₂, N[(C₁-C₆)-alkyl] {[(C₁-C₆)-alkyl]-SO₃H},
where the alcohol (OH) functions may be replaced by F and where the aryl or heteroaryl radical may be replaced by halogen, CN, (C₁-C₆)-alkyl, O—(C₁-C₆)-alkyl, OH, SO₂—NH₂, COOH, CONH₂, CO—O(C₁-C₆)-alkyl, CO—(C₁-C₆)-alkyl;
R18 is (CH₂)ₙ—CR25R26-CO—O(C₁-C₆)-alkyl, (CH₂)ₙ—CR25R26-CO—NH₂, (CH₂)ₙ—CR25R26-COOH;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
R23, R24 are each independently H, (C₁-C₆)-alkyl, (C₃-C₈)-cycloalkyl, [(C₁-C₆)-alkyl]-[(C₃-C₈)-cycloalkyl], aryl, [(C₁-C₆)-alkyl]-aryl
or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₆)-alkyl radical;
R25, R26 are each independently H, F, (C₁-C₆)-alkyl, aryl, [(C₁-C₆)-alkyl]-aryl, where the aryl may be substituted by halogen, CN, OH, O—(C₁-C₆)-alkyl, or the R25 and R26 radicals, together with the carbon atom bonded to them, form a three- to seven-membered carbocycle in which one carbon atom may be replaced by O, S(O)ₘ, NH, N[(C₁-C₆)-alkyl] or CO;
excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
and physiologically compatible salts thereof.

2. The compound of claim 1, wherein,
R, R′ are each independently H, (CH₂)ₙ-aryl, (C₁-C₆)-alkyl, where (C₁-C₆)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2, 3;
p is 1, 2, 3, 4;
q is 1, 2, 3;
r is 2, 3, 4, 5;
v is 0, 1, 2, 3;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, (CH₂)_{q}-[(C₃-C₈)-cycloalkyl], (CH₂)ₙ—[(C₇-C₁₂)-bicycloalkyl], (CH₂)ₙ—[(C₇-C₁₂)-tricycloalkyl], adamantan-1-yl, adamantan-2-yl, (CH₂)ₙ-aryl, (CH₂)ₙ-heteroaryl, OCF₃, O—R11, NR13R15, NH—CN, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₈)-cycloalkyl], CO—O—(CH₂)ₙ-aryl, CO—O—(CH₂)ₙ-heteroaryl, CO—NH₂, CO—NH—CN, CO—NH—[(C₁-C₈)-alkyl], CO—N[(C₁-C₈)-alkyl]₂, CO—NH—[(C₃-C₈)-cycloalkyl], C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₈)-alkyl, CO—(C₃-C₈)-cycloalkyl, CO-aryl, CO-heteroaryl, CH(OH)-aryl, CH(OH)-heteroaryl, CH[O—(C₁-C₆)-alkyl]-aryl, CH[O—(C₁-C₆)-alkyl]-heteroaryl, CHF-aryl, CHF-heteroaryl, CF₂-aryl, CF₂-heteroaryl, CHO, CH₂—OH, CH₂—CN, CH₂—O—R12, CH₂—O—(CH₂)_{q}—COOH, where the alkyl, cycloalkyl, cycloalkenyl, bicycloalkyl and tricycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, OCF₃, OH, O—(CH₂)ₙ-aryl, (CH₂)ₙ-aryl, S(O)ₘ—(C₁-C₆)-alkyl, SO₂—NH₂, SH, NR12R13, NH—CO—[(C₁-C₆)-alkyl], NH—CO—(CH₂)ₙ-aryl, (CH₂)ₙ—COOH, (CH₂)ₙ—CONH₂, (CH₂)ₙ—CO—O(C₁-C₆)-alkyl, (CH₂)ₙ—CO—(C₁-C₆)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)ᵣ—NH₂, (CH₂)ₙ—O—(CH₂)ₙ—CO—NH—(CH₂)ᵣ—OH, O—R13, OCF₃, (CH₂)ₙ—O—(CH₂)ᵣNH₂, (CH₂)ₙ—NH—R11, (CH₂)ₙ—N[(CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂ (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH[(C₃-C₈)-cycloalkyl], (CH₂)ₙ—NH—C(CH₃)₂—CO—NH[(C₃-C₈)-cycloalkyl], (CH₂)ₙ—NH—C(CH₃)₂—COOH, S(O)ₘ—R12, SO₂—R16, SO₂—N═CH—N(CH₃)₂, 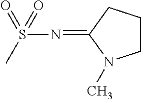
SO₂—NH—CO—R12, SO₂—NHR12, SO₂—NH—(CH₂), —OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)NH₂, (CH₂)ₙ—C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkenyl, (CH₂)ₙ—O—(C₃-C₈)-cycloalkyl, (CH₂)ₙ—O—(CH₂)_{q}-[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂)ᵣ—OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂)ᵣ—OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂, 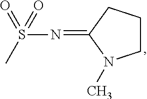
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], and where the alkyl radicals may be substituted by fluorine atoms;
where at least one of the R6, R7, R8, R9 and R10 radicals is always defined as T-bicyclic heterocycle, T-aryl or T-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23-C(═NR13)-NR24, NR23-C(═NR22)-NR24, CO—NR23-CR22R23, CO—CR22R23-CO, CR22R23-C0-CR22R24, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CO—NR23-SO₂, SO₂—NR23-CO, CO—CR22R23-SO₂, SO₂—CR22R23-CO, CR23R24-CR23R24-CR23R24, CR23R24-NR23-CR23R24;
R11 is H, (C₁-C₈)-alkyl, (C₂-C₁₀)-alkenyl, (C₂-C₁₀)-alkynyl, (C₃-C₈)-cycloalkyl, (CH₂)_{q}—[(C₃-C₈)-cycloalkyl], (CH₂)ₙ-aryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CR21[(CO—O(C₁-C₆)-alkyl)]₂, (CH₂)ₙ—CR21 (CONH₂)₂, (CH₂)ₙ—CR21 (COOH)₂, (CH₂)ₙ—CR21R22-CO—O[(C₁-C₆)-alkyl], (CH₂)ₙ—CR21R22-CONH₂, (CH₂)ₙ—CR21R22-CO—NH—[(C₁-C₈)-alkyl], (CH₂)ₙ—CR21R22-CO—N[C₁-C₈)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl, radicals may be substituted by fluorine atoms and where the aryl or heteroaryl radical may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl, and where the alkyl radicals may be substituted by fluorine atoms;
R12 is H, (C₁-C₈)-alkyl, (C₃-C₆)-cycloalkyl, (CH₂)ₙ-aryl, (CH₂)ₙ-heteroaryl, where the alkyl or cycloalkyl radicals may be substituted by fluorine atoms,
and where the aryl or heteroaryl radical may be substituted by halogen, CN, (C₁-C₆)-alkyl, O—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl,
where the alkyl and cycloalkyl radicals may be substituted by fluorine atoms and where the aryl or heteroaryl radical may be substituted by halogen, CN, CF₃, (C₁-C₆)-alkyl, (C₃-C₆)-cycloalkyl, O—[(C₁-C₆)-alkyl], S(O)ₘ—[(C₁-C₆)-alkyl], SO₂—NH₂, COOH, CONH₂, CO—[O(C₁-C₆)-alkyl], CO—(C₁-C₆)-alkyl, and where the alkyl radicals may be substituted by fluorine atoms;
R15 is (C₁-C₈)-alkyl,
where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholin-4-yl, thiomorpholine-1,1-dioxid-4-yl, NH—(CH₂)ᵣ—OH, NH—CH(CH₂OH)₂, NH—C(CH₂OH)₃, N[(C₁-C₆)-alkyl-OH]₂, D-glucamin-N-yl, N-methyl-D-glucamin-N-yl, NH—[(C₁-C₈)-alkyl]-CO—O(C₁-C₆)-alkyl, NH—[(C₁-C₈)-alkyl]-COOH, NH—[(C₁-C₈)-alkyl]-CONH₂, N[(C₁-C₆)-alkyl][(C₁-C₈)-alkyl]-COOH, NH—[C(H)(aryl)]-CO—O(C₁-C₆)-alkyl, NH-[C(H)(aryl)]-COOH, NH—[C(H)(aryl)]-CONH₂, NH—[C(H)(heteroaryl)]-CO—O(C₁-C₆)-alkyl, NH—[C(H)(heteroaryl)]-COOH, NH—[C(H)(heteroaryl)]-CONH₂, NH—[(C₃-C₈)-cycloalkyl]-CO—O(C₁-C₆)-alkyl, NH—[(C₃-C₈)-cycloalkyl]-COOH, NH—[(C₃-C₈)-cycloalkyl]-CONH₂, NH—(C₁-C₆)-alkyl-OH, NH—[(C₁-C₆)-alkyl]-SO₂—(C₁-C₆)-alkyl, NH—[(C₁-C₆)-alkyl]-S O₃H, NH—[(C₁-C₆)-alkyl]-SO₂—NH₂, where the alcohol (OH) functions may be replaced by F and where the aryl or heteroaryl radical may be replaced by halogen, CN, (C₁-C₆)-alkyl, O—(C₁-C₆)-alkyl, OH, SO₂—NH₂, COOH, CONH₂, CO—O(C₁-C₆)-alkyl, CO—(C₁-C₆)-alkyl;
R18 is (CH₂)ₙ—CR25R26—(O—O(C₁-C₄)-alkyl, (CH₂)ₙ—CR25R26-CO—NH₂, (CH₂)ₙ—CR25R26-COOH;
R20 is H, (C₁-C₆)-alkyl, (C₃-C₈)-cycloalkyl, aryl, [(C₁-C₆)-alkyl]-aryl;
R21 is H, F, CF₃, (C₁-C₆)-alkyl, (C₃-C₈)-cycloalkyl, OH, O—(C₁-C₆)-alkyl, O—(C₃-C₈)-cycloalkyl, O—(CH₂)ₙ-aryl, O—(CO)—(C₁-C₆)-alkyl, O—(CO)—(C₃-C₈)-cycloalkyl, O—(CO)—O—(C₁-C₆)-alkyl, O—(CO)—O—(C₃-C₈)-cycloalkyl, NH—[(C₁-C₆)-alkyl]-aryl, NH₂, NH—(C₁-C₆)-alkyl, NH—(CO)—(C₁-C₆)-alkyl;
R22 is H, CF₃, (C₁-C₆)-alkyl, aryl, [(C₁-C₆)-alkyl]-aryl;
R23, R24 are each independently H, (C₁-C₆)-alkyl, (C₃-C₆)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
and the physiologically compatible salts thereof.

3. The compound of claim 2, wherein,
R, R′ are each independently H, (CH₂)ₙ-aryl, (C₁-C₆)-alkyl where (C₁-C₆)-alkyl or the aryl radical may be substituted by halogen;
or R and R′ together form a ring having three to eight carbon atoms where one carbon atom may be replaced by O, S(O)ₘ, NR13 or NR15;
m is 0, 1, 2;
n is 0, 1, 2;
P
is 1, 2, 3;
q is 1, 2;
r is 2, 3, 4;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, adamantan-1-yl, adamantan-2-yl, (CH₂)ₙ-aryl, (CH₂)ₙ-heteroaryl, OCF₃, O—R11, NR13R15, S(O)ₘ—R12, SO₂—NH₂, SO₂—N═CH—N(CH₃)₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₆)-cycloalkyl], CO—NH₂, CO—NH—[(C₁-C₄)-alkyl], CO—N[(C₁-C₄)-alkyl]₂, CO—NH—[(C₃-C₆)-cycloalkyl], C(═NH)—O—[(C₁-C₆-alkyl)], C(═NH)—NH₂, C(═NH)—R16, C(═NR13)-NR12R13, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH,
where the alkyl and cycloalkyl radicals may be substituted by fluorine atoms, and where the aryl or heteroaryl radicals may be substituted by halogen, CN, (C₁-C₄)-alkyl, (C₃-C₆)-cycloalkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ₙ—OH, O—R13, OCF₃, (CH₂)ₙ—O—(CH₂)ᵣ—NH₂, (CH₂)ₙ—NH—R11, (CH₂)ₙ—N[CH₂)_{q}—CO—O(C₁-C₆)-alkyl]₂, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂ (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂, 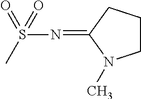
SO₂—NH—CO—R12, SO₂—NHR12, SO₂—NH—(CH₂)ᵣ—OH, SO₂—N[(C₁-C₈)-alkyl]₂, SO₂—NH—(CH₂), —NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)NH₂, (CH₂)ₙ—C(═NH)—NHOH, (CH₂)—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂; (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl, and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—O—(C₂-C₁₀)-alkynyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO—[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂), —OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂)ₙ—OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl]₂, (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO—[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂)ᵣOH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂—R16, SO₂—N═CH—N(CH₃)₂, 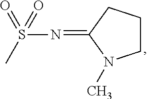
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl],
where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as T-bicyclic heterocycle, T-aryl or T-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T is NR23-CO—NR24, NR23-SO₂—NR24, SO₂—NR23-SO₂, CO—NR23-CO, NR23-C(═NR13)-NR24, NR23-C(═NR22)-NR24, CO—NR23-CR22R23, NR23-CO—CR22R24, NR23-SO₂—CR22R24, CR22R24-CO—NR23, CR22R24-SO₂—NR23, CR22R23-NR23-SO₂, SO₂—CR22R23-NR23, SO₂—NR23-CR22R23-, NR23-CR22R23-SO₂, CR23R24-CR23R24-CR23R24, CR23R24-NR23-CR23R24;
R11H, (C₁-C₈)-alkyl, (C₂-C₁₀)-alkynyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₆)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₄)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22-CO—O[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH,
where the alkyl, alkenyl, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms;
R12 is H, (C₁-C₈)-alkyl, (C₃-C₈)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl and where the alkyl radicals may be substituted by fluorine atoms;
R13 is H, SO₂—[(C₁-C₈)-alkyl], SO₂—[(C₃-C₈)-cycloalkyl], SO₂—(CH₂)ₙ-aryl, SO₂—(CH₂)ₙ-heteroaryl
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl] and where the alkyl radicals may be substituted by fluorine atoms;
R15 is (C₁-C₆)-alkyl,
where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholine-1,1-dioxid-4-yl, NH—(CH₂), —OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-alkyl][C₁-C₄)-alkyl]-COOH, NH—[C(H)(aryl)]-CO—O(C₁-C₄)-alkyl, NH—[C(H)(aryl)]-COOH, NH—[C(H)(aryl)]-CONH₂, NH-[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
where the alcohol (OH) functions may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 is (CH₂)ₙ—CR25R26-CO—O(C₁-C₄)-alkyl, (CH₂)ₙ—CR25R26-CO—NH₂, (CH₂)ₙ—CR25R26-COOH;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R23, R24 are each independently H, (C₁-C₄)-alkyl, (C₃-C₆)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
and the physiologically compatible salts thereof.

4. The compound of claim 3, wherein,
R, R′ are each independently H, aryl, (C₁-C₄)-alkyl, where (C₁-C₄)-alkyl or the aryl radical may be substituted by halogen; or R and R′ together form a ring having from three to eight carbon atoms, where one carbon atom may be replaced by O, S(O)ₘ, NR13 or NR15;
m is 0, 1, 2;
n is 0, 1, 2;
P
is 1, 2, 3;
q is 1, 2;
r is 2, 3;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 are defined as S or O and where the five-membered or six-membered ring may be fused to —(CH₂)₃— or —(CH₂)₄— or —CH═CH—CH═CH— to form a bicyclic system;
R1, R2, R3, R4, R5 are each independently H, F, Cl, Br, I, CN, CF₃, (C₁-C₈)-alkyl, (C₃-C₈)-cycloalkyl, (CH₂)ₙ-aryl, (CH₂)ₙ-heteroaryl, OCF₃, O—R11, NR13R15, S(O)ₘ—R12, SO₂—NH₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₆)-cycloalkyl], CO—NH₂, CO—NH—[(C₁-C₄)-alkyl], CO—N[(C₁-C₄)-alkyl]₂, C(═NH)—NH₂, C(═NH)—R16, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH, where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R6, R7, R8, R9, R10 are each independently T-bicyclic heterocycle, T-aryl or T-heteroaryl, where the bicyclic heterocycle or the aryl or heteroaryl radical may be fused to a 5- or 6-membered aromatic or nonaromatic carbon ring in which one or more CH or CH₂ groups may be replaced by oxygen atoms and where the 5- or 6-membered aromatic or nonaromatic carbon ring may be substituted by F, ═O or —(C₁-C₆)-alkyl and where the bicyclic heterocycle may contain from 9 to 12 ring members and up to five CH or CH₂ groups may each independently be replaced by N, NR20, O, S(O)ₘ or C═O and where the aryl or heteroaryl radical or bicyclic heterocycle may be unsubstituted or mono- or polysubstituted by
R11, F, Cl, Br, I, CN, N₃, NC, NO₂, CF₃, (CH₂)ₙ—O—R11, (CH₂)ₙ—O—(CH₂)ᵣ—OH, (CH₂)ₙ—O—CH(CH₂OH)₂, (CH₂)ₙ—O—(CH₂)ₙ—CO—O—(CH₂)—NH₂, (CH₂)ₙ—O—(CH₂)ₙ—CO—NH—(CH₂)ᵣ—OH, O—R13, OCF₃, (CH₂)—O—(CH₂)ᵣ—NH₂, (CH₂)ₙ—NH—R11, (CH₂)ₙ—NRCH₂)_{q}—CO—O(C₁-C₆)-alkylh, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—CN, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NR12—C(═NR13)-NHR12, (CH₂)ₙ—NR12—C(═NR12)-NR12R13, (CH₂)ₙ—NH—(CH₂)ₙ—CO—O—(CH₂)ᵣ—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣOH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—(CH₂)ₙ—CO—N[(C₃-C₈)-cycloalkyl]₂, (CH₂)ₙ—NH—C(CH₃)₂—CO—O(C₁-C₈)-alkyl, (CH₂)ₙ—NH—C(CH₃)₂—CO—O(C₃-C₈)-cycloalkyl, (CH₂)ₙ—NH—C(CH₃)₂—CO—O—(CH₂)ᵣ—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₈)-alkyl], (CH₂)ₙ—NH—C(CH₃)₂—CO—NH—(CH₂)ᵣ—OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—NH—C(CH₃)₂—COOH, S(O)ₘ—R12, SO₂—R16, SO₂—N═CH—N(CH₃)₂, 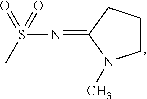
SO₂—NH—CO—R12, SO₂—NHR12, SO₂—NH—(CH₂)—OH, SO₂—N[(C₁-C₈)-alkyl]₂, SO₂—NH—(CH₂)ᵣ—NH₂, SF₅, COOH, CO—NH₂, (CH₂)_{q}—CN, (CH₂)ₙ—CO—NH—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—CHO, (CH₂)ₙ—C(═NH)—NH₂, (CH₂)ₙ—C(═NH)—NHOH, (CH₂)ₙ—C(═NH)—[NH—O—(C₁-C₆)-alkyl], (CH₂)ₙ—C(═NH)(R16), (CH₂)ₙ—C(═NR13)NHR12, (CH₂)ₙ—C(═NR12)NR12R13, (CH₂)ₙ—C(═NSO₂—R12)NH₂, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—O(C₁-C₆)-alkyl, CO—(C₁-C₆)-alkyl and where the alkyl radicals may be substituted by fluorine atoms,
and where, when R3 is CN, NO₂ or halogen and R4 is CF₃ or halogen and R and R′ are each methyl, the X-aryl radical is provided with at least one of the abovementioned substituents other than hydrogen;
H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-Cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—(C₂-C₁₀)-alkenyl, (CH₂)ₙ—O—(C₃-C₈)-cycloalkyl, (CH₂)ₙ—O—(CH₂)_{q}—[(C₃-C₈)-cycloalkyl], (CH₂)ₙ—O—(CH₂)ₙ—CO—[O—(C₁-C₈)-alkyl], (CH₂)ₙ—O—(CH₂)ₙ—CO[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂), —OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂)ₙ—OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO[O—(CH₂)ᵥ-aryl], (CH₂)ₙ—NH—(CH₂)ₙ—CO—[O—(CH₂)ₙ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₘ—(C₃-C₈)-cycloalkyl, (CH₂)ₙ—SO₂R16, SO₂—N═CH—N(CH₃)₂, 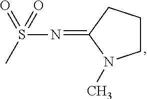
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl and where the alkyl radicals may be substituted by fluorine atoms;
where at least one of the R6, R7, R8, R9 and R10 radicals is always defined as T-bicyclic heterocycle, T-aryl or T-heteroaryl and
where one of the four radical pairs of R6 and R7, or R7 and R8, or R8 and R9, or R9 and R10 may in each case together form the —CH₂—CH₂—CH₂— or —CH₂—CH₂—CH₂—CH₂— groups in which up to two —CH₂— groups may be replaced by —O— and where the —CH₂—CH₂—CH₂— or —CH₂—CH₂—CH₂—CH₂— groups may be substituted by F, (C₁-C₈)-alkyl or ═O;
T NR23-CO—NR24, NR23-SO₂—NR24, SO₂—NR23-SO₂, CO—NR23-CO, NR23—C(═NR13)-NR24, NR23-C(═NR22)-NR24, CO—NR23-CR22R23, NR23-SO₂—CR22R24, CR22R24-SO₂—NR23, CR22R23-NR23-SO₂, SO₂—CR22R23-NR23, SO₂—NR23-CR22R23-, NR23-CR22R23-SO₂, CR23R24-CR23R24-CR23R24;
R11H, (C₁-C₈)-alkyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-CO—O[(C₁-C₄)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22-CO—O—[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH,
where the alkyl, alkeny, alkynyl and cycloalkyl radicals may be substituted by fluorine atoms and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl,
where the alkyl and cycloalkyl radicals may be substituted by fluorine atoms and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl] and where the alkyl radicals may be substituted by fluorine atoms;
R15 (C₁-C₆)-Alkyl,
where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholine-1,1-dioxid-4-yl, NH—(CH₂), —OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-Alkyl][(C₁-C₄)-Alkyl]-COOH, NH—[C(H)(aryl)]-CO—O(C₁-C₄)-alkyl, NH—[C(H)(aryl)]-COOH, NH—[C(H)(aryl)]-CONH₂, NH—[C(H)(heteroaryl)]-CO—O(C₁-C₄)-alkyl, NH—[C(H)(heteroaryl)]-COOH, NH—[C(H)(heteroaryl)]-CONH₂, NH—[(C₃-C₆)-cycloalkyl]-CO—O(C₁-C₄)-alkyl, NH—[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
where the alcohol (OH) functionalities may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 (CH₂)ₙ—CR25R26—CO—O(C₁-C₄)-alkyl, (CH₂)ₙ—CR25R26-CO—NH₂, (CH₂)ₙ—CR25R26-COOH;
R20 is H, (C₁-C₄)-alkyl, (C₃-C₆)-cycloalkyl, aryl, [(C₁-C₄)-alkyl]-aryl;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R23, R24 are each independently H, (C₁-C₄)-alkyl, (C₃-C₆)-cycloalkyl, [(C₁-C₄)-alkyl]-[(C₃-C₆)-cycloalkyl], aryl, [(C₁-C₄)-alkyl]-aryl or R23 and R24 together form a —CH═CH—, —CH₂—CH₂—, —CH₂—CH₂—CH₂— or —CH₂—CH₂—CH₂—CH₂— unit in which one CH₂ moiety may be replaced by C═O, CHF or CF₂, and in which up to four hydrogen atoms may be replaced by a (C₁-C₄)-alkyl radical;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
and the physiologically compatible salts thereof

5. A compound of the formula Ia 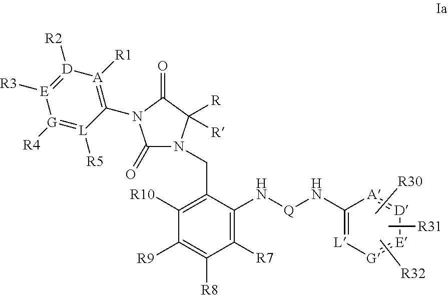
in which
R, R′ are each independently H, aryl, (C₁-C₄)-alkyl, where (C₁-C₄)-alkyl or the aryl radical may be substituted by halogen;
or R and R′ together form a ring having from three to eight carbon atoms, where one carbon atom may be replaced by O, S(O)ₘ, NR13 or NR15;
m is 0, 1, 2;
n is 0, 1, 2;
q is 1, 2;
r is 2, 3;
v is 0, 1, 2;
A, D, E, G, L are each independently C or N, where, when they are defined as N, the corresponding substituent R1, R2, R3, R4, R5 is absent, or R2-D=E-R3 or R4-G=L-R5 is defined as S or O and where the five- or six-membered ring may be fused to —(CH₂)₃— or —(CH₂)₄— or —CH═CH—CH═CH— to form a bicyclic system;
Q is C═O, SO₂;
R1, R2, R3, R4, R5 are each independently H, F, Cl, Br, I, CN, CF₃, (C₁-C₈)-alkyl, (C₃-C₈)-cycloalkyl, (CH₂)ₙ-aryl, (CH₂)ₙ-heteroaryl, OCF₃, O—R11, NR13R15, S(O)ₘ—R12, SO₂—NH₂, SO₂—NH—[(C₁-C₈)-alkyl], SO₂—NH—[(C₃-C₈)-cycloalkyl], SO₂—NH—(CH₂)ₙ-aryl, SO₂—NH—(CH₂)ₙ-heteroaryl, SO₂—N[(C₁-C₈)-alkyl]₂, SO₂—R16, SF₅, CO—O[(C₁-C₈)-alkyl], CO—O[(C₃-C₆)-cycloalkyl], CO—NH₂, CO—NH—[(C₁-C₄)-alkyl], CO—N[(C₁-C₄)-alkyl]₂, C(═NH)—NH₂, C(═NH)—R16, (CH₂)ₙ—C(═NSO₂—R12)NH₂, CO—R16, COOH, CO—(C₁-C₄)-alkyl, CO—(C₃-C₆)-cycloalkyl, CO-aryl, CO-heteroaryl, CH(OH)-aryl, CH(OH)-heteroaryl, CHF-aryl, CHF-heteroaryl, CF₂-aryl, CF₂-heteroaryl, CH₂—OH, CH₂—CN, CH₂—O—R12, CH₂—O—(CH₂)_{q}—COOH, where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, OCF₃, OH, O—(CH₂)ₙ-aryl, (CH₂)ₙ-aryl, S(O)ₘ—(C₁-C₄)-alkyl, SO₂—NH₂, SH, NR12R13, NH—CO—[(C₁-C₄)-alkyl], NH—CO—(CH₂)ₙ-aryl, (CH₂)ₙ—COOH, (CH₂)ₙ—CONH₂, (CH₂)ₙ—CO—O(C₁-C₄)-alkyl, (CH₂)ₙ—CO—(C₁-C₄)-alkyl, and where the alkyl radicals may be substituted by fluorine atoms;
R7, R8, R9, R10 are each independently H, F, Cl, Br, I, CN, N₃, NC, NO₂, CF₃, (C₁-C₈)-alkyl, (C₂-C₁₀)-alkenyl, (C₂-C₁₀)-alkynyl, (C₃-C₈)-cycloalkyl, aryl, heteroaryl, (CH₂)ₙ—CO—[O—(C₁-C₈)-alkyl], (CH₂)ₙ—CO—[O—(C₃-C₈)-cycloalkyl], (CH₂)ₙ—CO—[(C₁-C₈)-alkyl], (CH₂)ₙ—CO—[(C₃-C₈)-cycloalkyl], (CH₂)ₙ—CO—[O—(CH₂)ᵥ-aryl], (CH₂)ₙ—CO—NH₂, (CH₂)ₙ—COOH, (CH₂)ₙ—CO—NH—CN, (CH₂)ₙ—P(O)(OH)[O—(C₁-C₆)-alkyl], (CH₂)ₙ—P(O)[O—(C₁-C₆)-alkyl]₂, (CH₂)ₙ—P(O)(OH)(O—CH₂-aryl), (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₈)-alkyl], (CH₂)ₙ—CO—N[(C₁-C₈)-alkyl]₂, (CH₂)ₙ—CO—NH—[(C₃-C₈)-cycloalkyl], (C₂-C₁₀)-alkenyl-CO—O[(C₁-C₆)-alkyl], (C₂-C₁₀)-alkenyl-CONH₂, (C₂-C₁₀)-alkenyl-COOH, (C₂-C₁₀)-alkynyl-CO—O[(C₁-C₆)-alkyl], (C₂-C₁₀)-alkynyl-CONH₂, (C₂-C₁₀)-alkynyl-COOH, (CH₂)ₙ—CO—R16, (CH₂)ₙ—OH, (CH₂)ₙ—O—(C₁-C₈)-alkyl, (CH₂)ₙ—O—(C₂-C₁₀)-alkenyl, (CH₂)ₙ—CO—(C₂-C₁₀)-alkynyl, (CH₂)ₙ—O—(C₃-C₈)-cycloalkyl, (CH₂)ₙ—O—(CH₂)_{q}-[(C₃-C₈)-cycloalkyl]₂, (CH₂)ₙ—O—(CH₂)ₙ—CO—[O—(C₁-C₈)-alkyl]₂, (CH₂)ₙ—O—(CH₂)ₙ—CO—[O—(C₃-C₈)-cycloalkyl], (CH₂)ₙ—O—(CH₂)ₙ—CO—[(C₁-C₈)-alkyl], (CH₂)ₙ—O—(CH₂)ₙ—CO—[(C₃-C₈)-cycloalkyl], (CH₂)ₙ—O—(CH₂)ₙ—CO[O—(CH₂)ᵥ-aryl], (CH₂)ₙ—O—(CH₂)ₙ—CO—[O—(CH₂)ᵥ-heteroaryl], (CH₂)ₙ—O—(CH₂)_{q}—CO—NH₂, (CH₂)ₙ—O—(CH₂)_{q}—COOH, (CH₂)ₙ—O—(CH₂)_{q}—CO—NH—CN, (CH₂)ₙ—O—(CH₂)ₙ—P(O)(OH)[O—(C₁-C₆)-alkyl], (CH₂)ₙ—O—(CH₂)ₙ—P(O)[O—(C₁-C₆)-alkyl]₂, (CH₂)ₙ—O—(CH₂)ₙ—P(O)(OH)(O—CH₂-aryl), (CH₂)ₙ—O—(CH₂)ₙ—P(O)(O—CH₂-aryl)₂, (CH₂)ₙ—O—(CH₂)ₙ—P(O)(OH)₂, (CH₂)ₙ—O—(CH₂)ₙ—SO₃H, (CH₂)ₙ—O—(CH₂)ₙ—SO₂—NH₂, (CH₂)ₙ—O—(CH₂)ₙ—CO—NH—[(C₁-C₈)-alkyl], (CH₂)ₙ—O—(CH₂)ₙ—CO—N[(C₁-C₈)-alkyl]₂, (CH₂)ₙ—O—(CH₂)ₙ—CO—NH—[(C₃-C₈)-cycloalkyl], (CH₂)ₙ—O—(CH₂)ₙ—CR21R22-CO—O—[(C₁-C₆)-alkyl], (CH₂)ₙ—O—(CH₂)ₙ—CR21R22-CONH₂, (CH₂)ₙ—O—(CH₂)ₙ—CR21R22-COOH, (CH₂)ₙ—O—(CH₂)ₙ—CO—R16, (CH₂)ₙ—O—(CH₂), —OH, (CH₂)ₙ—O—CH(CH₂OH)₂, (CH₂)ₙ—O—(CH₂)ₙ—CO—O—(CH₂), —NH₂, (CH₂)ₙ—O—(CH₂)ₙ—CO—NH—(CH₂), —OH, O—R13, OCF₃, (CH₂)ₙ—NH₂, (CH₂)ₙ—NH—(C₁-C₈)-alkyl, (CH₂)ₙ—NH—(C₃-C₈)-cycloalkyl, (CH₂)ₙ—NH—(CH₂)ₙ—CO—[O—(C₃-C₈)-cycloalkyl], (CH₂)ₙ—NH—(CH₂)ₙ—CO—[(C₁-C₈)-alkyl], (CH₂)ₙ—NH—(CH₂)ₙ—CO—[(C₃-C₈)-cycloalkyl], (CH₂)ₙ—NH—(CH₂)ₙ—CO[O—(CH₂)ᵥ-aryl], (CH₂)ₙ—NH—(CH₂)ₙ—CO—[O—(CH₂)ᵥ-heteroaryl], (CH₂)ₙ—NH—(CH₂)_{q}—CO—NH—CN, (CH₂)ₙ—NH—(CH₂)ₙ—P(O)(OH)₂, (CH₂)ₙ—NH—(CH₂)ₙ—SO₃H, (CH₂)ₙ—NH—(CH₂)ₙ—SO₂—NH₂, (CH₂)ₙ—NH—(CH₂)ₙ—CR21R22-CO—O—[(C₁-C₆)-alkyl], (CH₂)ₙ—NH—(CH₂)ₙ—CR21R22-CONH₂, (CH₂)ₙ—NH—(CH₂)ₙ—CR21R22-COOH, (CH₂)ₙ—NH—(CH₂)ₙ—CO—R16, (CH₂)ₙ—NH—(CH₂)ₙ—SO₂—[(C₁-C₈)-alkyl], (CH₂)ₙ—NH—(CH₂)ₙ—SO₂—[(C₃-C₈)-cycloalkyl], (CH₂)ₙ—NH—SO₂—(CH₂)ₙ—NH₂, (CH₂)ₙ—NH—SO₂—(CH₂)ₙ—NH—(C₁-C₈)-alkyl, (CH₂)ₙ—NH—SO₂—(CH₂)ₙ—NH—(C₃-C₈)-cycloalkyl, (CH₂)ₙ—NH—SO₂—(CH₂)ₙ—N[(C₁-C₈)-alkyl]₂, (CH₂)ₙ—NH—CN, (CH₂)ₙ—NH—SO₂—R16, (CH₂)ₙ—NR12—CO—NH—(C₁-C₈)-alkyl, (CH₂)ₙ—NR12—CO—NH—(C₃-C₈)-cycloalkyl, (CH₂)ₙ—NR12—CO—NH₂, (CH₂)ₙ—NR12—CO—NH—SO₂—(C₁-C₈)-alkyl, (CH₂)ₙ—NR12—CO—NH—SO₂—(C₃-C₈)-cycloalkyl, (CH₂)ₙ—NR12—CO—N[(C₁-C₈)-alkyl]₂, (CH₂)ₙ—NH—CO—NH—(CH₂)ₙ—CO—[O—(C₁-C₈)-alkyl], (CH₂)ₙ—NH—CO—NH—(CH₂)_{q}—CO—NH₂, (CH₂)ₙ—NH—CO—NH—(CH₂)_{q}—COOH, (CH₂)ₙ—NH—C(═NH)—NH₂, (CH₂)ₙ—NH—C(═NH)—R16, (CH₂)ₙ—NH—C(═NH)—NH[(C₁-C₈)-Alkyl], (CH₂)ₙ—NH—C(═N—SO₂—(C₁-C₈)-alkyl)-NH₂, (CH₂)ₙ—NH—C(═N—SO₂—(C₁-C₈)-alkyl)-NH[(C₁-C₈)-alkyl], (CH₂)ₙ—NH—C(═N—SO₂—NH₂)—NH₂, (CH₂)ₙ—NH—C(═N—SO₂—NH₂)—NH[(C₁-C₈)-alkyl], (CH₂)ₙ—NH—C(═NH)—N[(C₁-C₈)-alkyl]₂, (CH₂)ₙ—NH—C(═N—SO₂—(C₁-C₈)-alkyl)-N[(C₁-C₈)-alkyl]₂, (CH₂)ₙ—NH—(CH₂)ₙ—CO—O—(CH₂)—NH₂, (CH₂)ₙ—NH—(CH₂)ₙ—CO—NH—[(C₁-C₈)-alkyl], (CH₂)ₙ—NH—(CH₂)ₙ—CO—NH—(CH₂)ᵣ—OH, (CH₂)ₙ—NH—(CH₂)ₙ—CO—N[(C₁-C₈)-alkyl]₂, (CH₂)ₙ—NH—(CH₂)ₙ—CO—NH—[(C₃-C₈)-cycloalkyl], (CH₂)ₙ—NH—C(CH₃)₂—CO—O(C₃-C₈)-cycloalkyl, (CH₂)ₙ—NH—C(CH₃)₂—CO—O—(CH₂)—NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH—[(C₁-C₈)-alkyl], (CH₂)ₙ—NH—C(CH₃)₂—CO—NH—(CH₂), —OH, (CH₂)ₙ—NH—C(CH₃)₂—CO—N[(C₁-C₈)-alkyl]₂, (CH₂)ₙ—NH—(CH₃)₂—CO—NH—[(C₃-C₈)-cycloalkyl], (CH₂)ₙ—NH—C(CH₃)₂—CO—N[(C₃-C₈)-cycloalkyl]₂, (CH₂)ₙ—S(O)ₘ—(C₁-C₈)-alkyl, (CH₂)ₙ—S(O)ₙ—(C₁-C₈)-cycloalkyl, (CH₂)ₙ—SO₂—R16, SO₂—N═CH—N(CH₃)₂, 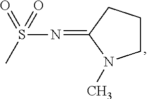
(CH₂)ₙ—SO₂—NH—CO—(C₁-C₈)-alkyl, (CH₂)ₙ—SO₂—NH—CO—(C₃-C₈)-cycloalkyl, (CH₂)ₙ—SO₂—NH—(C₁-C₈)-alkyl, (CH₂)ₙ—SO₂—NH—(C₃-C₈)-cycloalkyl, (CH₂)ₙ—SO₂—N[(C₁-C₈)-alkyl]₂, SO₂—NH—(CH₂), —OH, SO₂—NH—(CH₂), —NH₂, SF₅, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—(C₁-C₈)-alkyl, (CH₂)ₙ—CO—NH—SO₂—(C₃-C₈)-cycloalkyl, (CH₂)ₙ—CHO, (CH₂)ₙ—C(═NH)NH₂, (CH₂)ₙ—C(═NH)NHOH, (CH₂)ₙ—C(═NH)(R16), (CH₂)ₙ—C(═NR13)NHR12, (CH₂)ₙ—C(═NR12)NR12R13, (CH₂)ₙ—C(═NH)O[(C₁-C₆)-alkyl], where the alkyl and cycloalkyl radicals may be substituted by fluorine atoms, and where the aryl or heteroaryl radicals may be substituted by halogen, CN, (C₁-C₆)-alkyl, (C₃-C₆)-cycloalkyl, O—(C₁-C₆)-alkyl, S(O)ₘ—(C₁-C₆)-alkyl, SO₂—NH₂, COOH, CONH₂, CO—[O(C₁-C₆)-alkyl], CO—(C₁-C₆)-alkyl, and where the alkyl radicals may be substituted by fluorine atoms;
where one of the three radical pairs of R7 and R8, or R8 and R9, or R9 and R10 may in each case together form the —CH₂—CH₂—CH₂— or —CH₂—CH₂—CH₂—CH₂— groups in which up to two —CH₂ groups may be replaced by —O— and where the —CH₂—CH₂—CH₂— or —CH₂—CH₂—CH₂—CH₂-groups may be substituted by F, (C₁-C₈)-alkyl or ═O;
R11 is H, (C₁-C₈)-alkyl, (C₃-C₆)-cycloalkyl, (CH₂)ₙ-aryl, (CH₂)ₙ—CO—[O—(C₁-C₆)-alkyl], (CH₂)ₙ—CO—[O—(C₃-C₆)-cycloalkyl], (CH₂)ₙ—CO—[(C₁-C₆)-alkyl], (CH₂)ₙ—CO—[(C₃-C₆)-cycloalkyl], (CH₂)ₙ—CO-aryl, (CH₂)ₙ—CO-heteroaryl, (CH₂)_{q}—CO—NH₂, (CH₂)_{q}—COOH, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, (CH₂)ₙ—SO₃H, (CH₂)ₙ—SO₂—NH₂, (CH₂)ₙ—CO—NH—[(C₁-C₄)-alkyl], (CH₂)ₙ—CO—N[(C₁-C₄)-alkyl]₂, (CH₂)ₙ—CO—NH—[(C₃-C₆)-cycloalkyl], (C₂-C₆)-alkenyl-CO—O[(C₁-C₄)-alkyl], (C₂-C₆)-alkenyl-CONH₂, (C₂-C₆)-alkenyl-COOH, (C₂-C₆)-alkynyl-00-0[(C₁-C₄)-alkyl], (C₂-C₆)-alkynyl-CONH₂, (C₂-C₆)-alkynyl-COOH, (CH₂)ₙ—CR21[(CO—O(C₁-C₄)-alkyl)]₂, (CH₂)ₙ—CR21(CONH₂)₂, (CH₂)ₙ—CR21(COOH)₂, (CH₂)ₙ—CR21R22-CO—O—[(C₁-C₄)-alkyl], (CH₂)ₙ—CR21R22-CONH₂, (CH₂)ₙ—CR21R22-CO—NH—[(C₁-C₄)-alkyl], (CH₂)ₙ—CR21R22-CO—N[(C₁-C₄)-alkyl]₂, (CH₂)ₙ—CR21R22-COOH, (CH₂)ₙ—CO—R16, (CH₂)ₙ—CO—NH—C(CH₃)₂—CO—O[(C₁-C₈)-alkyl], (CH₂)ₙ—CO—NH—C(CH₃)₂—CONH₂, (CH₂)ₙ—CO—NH—C(CH₃)₂—COOH, where the alkyl, alkenyl, alkynyl and cycloalkyl radicals may be substituted by fluorine atoms, and where the aryl or heteroaryl radical may be substituted by halogen, CN, (C₁-C₄)-alkyl, O—(C₁-C₄)-alkyl, S(O)ₘ—(C₁-C₄)-alkyl, SO₂—NH₂, COOH, CONH₂, CO—O(C₁-C₄)-alkyl, and where the alkyl radicals may be substituted by fluorine atoms;
R12 is H, (C₁-C₄)-alkyl, (C₃-C₆)-cycloalkyl, (CH₂)ₙ-aryl, (CH₂)ₙ-heteroaryl, where the alkyl or cycloalkyl radicals may be substituted by fluorine atoms, and where the aryl or heteroaryl radical may be substituted by halogen, CN, (C₁-C₄)-alkyl, O—(C₁-C₄)-alkyl, SO₂—NH₂, COOH, CONH₂, CO—O(C₁-C₄)-alkyl, and where the alkyl radicals may be substituted by fluorine atoms;
R13 is H, SO₂—[(C₁-C₄)-alkyl], SO₂—[(C₃-C₆)-cycloalkyl], SO₂—(CH₂)ₙ-aryl, SO₂—(CH₂)ₙ-heteroaryl,
where the alkyl and cycloalkyl radicals may be substituted by fluorine atoms, and where the aryl or heteroaryl radical may be substituted by halogen, CN, CF₃, (C₁-C₄)-alkyl, O—[(C₁-C₄)-alkyl], S(O)ₘ—[(C₁-C₆)-alkyl], SO₂—NH₂, COOH, CONH₂, CO—[O(C₁-C₄)-alkyl], and where the alkyl radicals may be substituted by fluorine atoms;
R15 is (C₁-C₆)-alkyl,
where the alkyl radical may be substituted by fluorine atoms;
R16 is aziridin-1-yl, azetidin-1-yl, 3-hydroxyazetidin-1-yl, piperidin-1-yl, pyrrolidin-1-yl, 3-pyrrolidino1-1-yl, morpholin-N-yl, piperazin-1-yl, 4-[(C₁-C₆)-alkyl]piperazin-1-yl, thiomorpholin-1,1-dioxid-4-yl, NH—(CH₂), —OH, NH—CH(CH₂OH)₂, NH—C(CH₂OH)₃, N[(C₁-C₄)-alkyl-OH]₂, D-glucamin-N-yl, N-methyl-D-glucamin-N-yl, NH—[(C₁-C₄)-alkyl]-COOH, NH—[(C₁-C₄)-alkyl]-CONH₂, N[(C₁-C₄)-alkyl][(C₁-C₄)-alkyl]-COOH, NH—[C(H)(aryl)]-CO—O(C₁-C₄)-alkyl, NH—[C(H)(aryl)]-COOH, NH—[C(H)(aryl)]-CONH₂, NH—[C(H)(heteroaryl)]-CO—O(C₁-C₄)-alkyl, NH—[C(H)(heteroaryl)]-COOH, NH-[C(H)(heteroaryl)]-CONH₂, NH—[(C₃-C₆)-cycloalkyl]-CO—O(C₁-C₄)-alkyl, NH-[(C₃-C₆)-cycloalkyl]-COOH, NH—[(C₃-C₆)-cycloalkyl]-CONH₂, NH—(C₁-C₄)-alkyl-OH, NH—[(C₁-C₄)-alkyl]-SO₂—(C₁-C₄)-alkyl, NH—[(C₁-C₄)-alkyl]-SO₃H, NH—[(C₁-C₄)-alkyl]-SO₂—NH₂,
where the alcohol (OH) functions may be replaced by F and where the aryl or heteroaryl radical may be substituted by halogen, CN, (C₁-C₄)-alkyl, O—(C₁-C₄)-alkyl, OH, SO₂—NH₂, COOH, CONH₂, CO—O(C₁-C₄)-alkyl;
R18 is (CH₂)ₙ—CR25R26-CO—O(C₁-C₄)-alkyl, (CH₂)ₙ—CR25R26-CO—NH₂, (CH₂)ₙ—CR25R26-COOH;
R21 is H, F, CF₃, (C₁-C₄)-alkyl, (C₃-C₆)-cycloalkyl, OH, O—(C₁-C₄)-alkyl, O—(C₃-C₆)-cycloalkyl, O—(CH₂)ₙ-aryl, O—(CO)—(C₁-C₄)-alkyl, O—(CO)—(C₃-C₆)-cycloalkyl, O—(CO)—O—(C₁-C₄)-alkyl, O—(CO)—O—(C₃-C₆)-cycloalkyl, NH—[(C₁-C₄)-alkyl]-aryl, NH₂, NH—(C₁-C₄)-alkyl, NH—(CO)—(C₁-C₄)-alkyl;
R22 is H, CF₃, (C₁-C₄)-alkyl, aryl, [(C₁-C₄)-alkyl]-aryl;
R25, R26 are each independently H, F, (C₁-C₄)-alkyl, aryl, [(C₁-C₄)-alkyl]-aryl, where the aryl may be substituted by halogen, CN, OH, O—(C₁-C₄)-alkyl, or the R25 and R26 radicals, together with the carbon atom bonded to them, form a three- to seven-membered carbocycle in which one carbon atom may be replaced by O, S(O)ₘ, NH, N[(C₁-C₄)-alkyl] or CO;
A′, D′, E′, G′, L′ are each independently CH or N, where, when they are defined as N, the corresponding substituent R30, R31 or R32 is absent if it would be bonded to the nitrogen atom;
R30, R31, R32 are each independently R11, F, Cl, Br, I, CN, CF₃, (CH₂)ₙ—O—R11, O—R13, OCF₃, (CH₂)ₙ—NH—R11, (CH₂)ₙ—NRCH₂)_{q}—CO—O(C₁-C₄)-alkylh, (CH₂)ₙ—N[(CH₂)_{q}—COOH]₂, (CH₂)ₙ—N[(CH₂)_{q}—CONH₂]₂, (CH₂)ₙ—NH—R13, (CH₂)ₙ—N(R13)₂, (CH₂)ₙ—NH—SO₂—R16, (CH₂)ₙ—NH—(CH₂)ₙ—SO₂—R12, (CH₂)ₙ—NR12—CO—R16, (CH₂)ₙ—NR12—CO—NR12R13, (CH₂)ₙ—NR12—CO—N(R12)₂, (CH₂)ₙ—NR12—CO—NHR11, (CH₂)ₙ—NH—C(═NH)—NH₂, (CH₂)ₙ—NH—C(═NH)—R16, (CH₂)ₙ—NH—C(═NH)—NHR12, (CH₂)ₙ—NH—(CH₂)ₙ—CO—NH—[(C₁-C₄)-alkyl], (CH₂)ₙ—NH—(CH₂)ₙ—CO—N[(C₁-C₄)-alkyl]₂, (CH₂)ₙ—NH—C(CH₃)₂—CO—O(C₁-C₄)-alkyl, (CH₂)ₙ—NH—C(CH₃)₂—CO—O(C₃-C₆)-cycloalkyl, (CH₂)ₙ—NH—C(CH₃)₂—CO—O—(CH₂), —NH₂, (CH₂)ₙ—NH—C(CH₃)₂—CO—O—(CH₂)ₙ-aryl, (CH₂)ₙ—NH—C(CH₃)₂—CO—O—(CH₂)ₙ-heteroaryl, (CH₂)ₙ—NH—C(CH₃)₂—CO—NH₂, (CH₂)ₙ—NH—C(CH₃)₂—CO—NH—[(C₁-C₄)-alkyl], (CH₂)ₙ—NH—C(CH₃)₂—CO—N[(C₁-C₄)-alkyl]₂, (CH₂)ₙ—NH—C(CH₃)₂—COOH, S(O)ₘ—R12, SO₂—R16, SO₂—N═CH—N(CH₃)₂, SO₂—NH—CO—R12, SO₂—NHR12, SO₂—N[(C₁-C₄)-alkyl]₂, SF₅, COOH, CO—NH₂, (CH₂)_{q}—CN, (CH₂)ₙ—CO—NH-piperidin-1-yl, (CH₂)ₙ—CO—NH—SO₂—NHR12, (CH₂)ₙ—CO—NH—SO₂—R18, (CH₂)ₙ—C(═NH)—NHOH, (CH₂)ₙ—C(═NR13)NHR12, (CH₂)ₙ—C(═NR12)NR12R13, (CH₂)ₙ—C(═NSO₂—R12)NH₂, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(O—CH₂-aryl)₂, (CH₂)ₙ—P(O)(OH)₂, where the alkyl and cycloalkyl radicals may be substituted by fluorine atoms, and where the aryl or heteroaryl radicals may be substituted by halogen, CN, (C₁-C₄)-alkyl, O—(C₁-C₄)-alkyl, S(O)ₘ—(C₁-C₄)-Alkyl, SO₂—NH₂, COOH, CONH₂, CO—O(C₁-C₄)-alkyl, and where the alkyl radicals may be substituted by fluorine atoms;
excluding the compound N-[3-t-butyl-1-(4-methylphenyl)-1H-pyrazol-5-yl]-N′-(4-{[3-(2-methylphenyl)-2,4-dioxo-1-imidazolidinyl]methyl]}phenyl)urea,
and the physiologically compatible salts thereof.

6. The compound of claim 5, wherein,
R, R′ are each (C₁-C₄)-alkyl;
or R and R′ together form a ring having from three to eight carbon atoms;
m is 0, 1, 2;
n is 0, 1, 2;
A, D, E, G, L are each C;
Q is C═O, SO₂;
R1, R2, R3, R4, R5 are each independently H, F, Cl, Br, CN, CF₃, (C₁-C₈)-cycloalkyl, O-phenyl;
R7, R8, R9, R10 are each independently H, F, Cl, Br, CF₃, —OCH₃;
A′, E′ are each independently CH or N, where, when they are defined as N, the corresponding substituent R30, R31 or R32 is absent if it would be bonded to the nitrogen atom,
R30, R31, R32 are each independently H, F, Cl, Br, CF₃, OH, NO₂, NH₂, CONH₂, COOH, —COO—(C₁-C₈)-alkyl, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, S(O)ₘ—(C₁-C₄)-alkyl, S(O)ₘ—(CH₂)—COOH, S(O)ₘ—(CH₂)—COO—(C₁-C₄)-alkyl, SO₂—Cl, SO₂—NH₂, SO₃H;
and the physiologically compatible salts thereof.

7. The compound of claim 6, wherein,
R, R′ are each (C₁-C₄)-alkyl;
or R and R′ together form a ring having from three to eight carbon atoms;
m is 0, 1, 2;
n is 0, 1, 2;
A, D, E, G, L are each C;
Q is C═O, SO₂;
R1, R2, R3, R4, R5 are each independently H, F, Cl, Br, CN, CF₃, (C₁-C₈)-cycloalkyl, O-phenyl;
R7, R8, R9, R10 are each independently H, F, Cl, Br, CF₃, —OCH₃;
A′, E′ are each independently CH or N, where, when they are defined as N, the corresponding substituent R30, R31 or R32 is absent if it would be bonded to the nitrogen atom,
R30, R31, R32 are each independently H, F, Cl, Br, CF₃, OH, NO₂, NH₂, CONH₂, COOH, —COO—(C₁-C₈)-alkyl, (CH₂)ₙ—P(O)(OH)[O—(C₁-C₄)-alkyl], (CH₂)ₙ—P(O)[O—(C₁-C₄)-alkyl]₂, (CH₂)ₙ—P(O)(OH)₂, S(O)ₘ—(C₁-C₄)-alkyl, S(O)ₘ—(CH₂)—COOH, S(O)ₘ—(CH₂)—COO—(C₁-C₄)-alkyl, SO₂—Cl, SO₂—NH₂, SO₃H;
and the physiologically compatible salts thereof.

8. A pharmaceutical composition comprising a pharmaceutically acceptable amount of the compound of claim 1.

9. A pharmaceutical composition comprising a pharmaceutically acceptable amount of the compound of claim 5.

10. The pharmaceutical composition of claim 8 and a pharmaceutically acceptable carrier.

11. The pharmaceutical composition of claim 9 and a pharmaceutically acceptable carrier.

12. The pharmaceutical composition of claim 10 and at least one further active ingredient.

13. The pharmaceutical composition of claim 11 and at least one further active ingredient.

14. The pharmaceutical composition as claimed in claim 12, which comprises, as a further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY antagonists, MC4 agonists, orexin antagonists, H3 antagonists, TNF agonists, CRF antagonists, CRF BP antagonists, urocortin agonists, β3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β-agonists or amphetamines.

15. The pharmaceutical composition as claimed in claim 13, which comprises, as a further active ingredient, one or more antidiabetics, active hypoglycemic ingredients, HMG-CoA reductase inhibitors, cholesterol absorption inhibitors, PPAR gamma agonists, PPAR alpha agonists, PPAR alpha/gamma agonists, PPAR delta agonists, fibrates, MTP inhibitors, bile acid absorption inhibitors, MTP inhibitors, CETP inhibitors, polymeric bile acid adsorbers, LDL receptor inducers, ACAT inhibitors, antioxidants, lipoprotein lipase inhibitors, ATP citrate lyase inhibitors, squalene synthetase inhibitors, lipoprotein(a) antagonists, HM74A receptor agonists, lipase inhibitors, insulins, sulfonylureas, biguanides, meglitinides, thiazolidinediones, α-glucosidase inhibitors, active ingredients which act on the ATP-dependent potassium channel of the beta cells, glycogen phosphorylase inhibitors, glucagon receptor antagonists, activators of glucokinase, inhibitors of gluconeogenesis, inhibitors of fructose 1,6-biphosphatase, modulators of glucose transporter 4, inhibitors of glutamine-fructose-6-phosphate amidotransferase, inhibitors of dipeptidylpeptidase IV, inhibitors of 11-beta-hydroxysteroid dehydrogenase 1, inhibitors of protein tyrosine phosphatase 1B, modulators of the sodium-dependent glucose transporter 1 or 2, modulators of GPR40, inhibitors of hormone-sensitive lipase, inhibitors of acetyl-CoA carboxylase, inhibitors of phosphoenolpyruvate carboxykinase, inhibitors of glycogen synthase kinase-3 beta, inhibitors of protein kinase C beta, endothelin-A receptor antagonists, inhibitors of I kappaB kinase, modulators of the glucocorticoid receptor, CART agonists, NPY antagonists, MC4 agonists, orexin antagonists, H3 antagonists, TNF agonists, CRF antagonists, CRF BP antagonists, urocortin agonists, P3 agonists, CB1 receptor antagonists, MSH (melanocyte-stimulating hormone) agonists, MCH antagonists, CCK agonists, serotonin reuptake inhibitors, mixed serotoninergic and noradrenergic compounds, 5HT modulators, bombesin agonists, galanin antagonists, growth hormones, growth hormone-releasing compounds, TRH agonists, decoupling protein 2 or 3 modulators, leptin agonists, DA agonists (bromocriptin, doprexin), lipase/amylase inhibitors, PPAR modulators, RXR modulators or TR-β-agonists or amphetamines.

16. A method of treatment of metabolic syndrome, diabetes, obesity, weight reduction, nicotine dependence, alcohol dependence, CNS disorders, schizophrenia or Alzheimer's comprising administering a pharmaceutically acceptable amount of the compound of claim 1 to a patient in need thereof.

17. A method of treatment of metabolic syndrome, diabetes, obesity, weight reduction, nicotine dependence, alcohol dependence, CNS disorders, schizophrenia or Alzheimer's comprising administering a pharmaceutically acceptable amount of the compound of claim 5 to a patient in need thereof.

18. A process for producing a pharmaceutical composition comprising one or more of the compounds of claim 1, which comprises mixing the active ingredient with a pharmaceutically suitable carrier and bringing this mixture into a form suitable for administration.

19. A process for producing a pharmaceutical composition comprising one or more of the compounds of claim 5, which comprises mixing the active ingredient with a pharmaceutically suitable carrier and bringing this mixture into a form suitable for administration.
